(19) United States
(12) Patent Application Publication
(10) Pub. No.: US 2017/0367963 A1
(43) Pub. Date: Dec. 28, 2017

(54) AMPHIPHILIC SUSPENSION AND STABILITY AGENT FOR ANTIDANDRUFF HAIR CARE COMPOSITIONS

(71) Applicant: Lubrizol Advanced Materials, Inc., Cleveland, OH (US)

(72) Inventors: Murat Kadir, Brecksville, OH (US); Neil Howard, Hudson, OH (US); Shui-Jen Raymond Hsu, Westlake, OH (US); Krishnan Chari, Hudson, OH (US)

(21) Appl. No.: 15/536,885

(22) PCT Filed: Dec. 16, 2015
(86) PCT No.: PCT/US2015/066021

§ 371 Date: Jun. 16, 2017

Related U.S. Application Data
(60) Provisional application No. 62/093,631, filed on Dec. 18, 2014.

(51) Int. Cl. A61K 8/81; A61K 8/27; A61K 8/04; A61K 8/49; A61Q 5/00; A61K 8/86

(52) U.S. Cl. CPC A61K 8/8152; A61Q 5/006; A61K 8/86; A61K 8/044; A61K 8/4933; A61K 8/27; A61K 2800/5422

(57) ABSTRACT

A hair care composition comprising: i) at least one crosslinked nonionic amphiphilic suspending polymer; ii) at least one anionic surfactant; iii) at least one particulate antidandruff agent; and iv) water. The suspending polymer is a pH independent nonionic, amphiphilic emulsion polymer that is crosslinked with an amphiphilic crosslinking agent and effectively suspends water insoluble particulate antidandruff agents.

### FIELD

Certain embodiments of the present technology relate to antidandruff hair care compositions including a suspension composition capable of achieving a substantial and unexpected reduction in the separation of insoluble materials, such as a particulate antidandruff agent, while maintaining acceptable viscosities and foaming properties. Additionally, certain embodiments of the present technology concern a phase stable aqueous surfactant containing hair care composition comprising a pH independent amphiphilic emulsion polymer capable of indefinitely suspending an insoluble antidandruff agent for topical delivery to the hair, scalp and skin.

### BACKGROUND

Numerous antidandruff hair care compositions such as antidandruff shampoos are commercially available or otherwise known in the art. These compositions typically comprise water, a detersive surfactant and a particulate antidandruff agent dispersed throughout the composition. Typical antidandruff agents used for this purpose include salicylic acid, sulfur, selenium sulfide or a polyvalent metal salt of pyrithione. These agents are most often insoluble or sparingly soluble in aqueous surfactant containing media, and are present in the antidandruff composition as discrete insoluble particulate solids. For example, zinc pyrithione is substantially insoluble in water (10-20 ppm). To ensure the consumer of an efficacious dose of antidandruff agent during each shampoo cycle, these particles should be homogeneously dispersed and suspended throughout the composition. Without a suspending agent the formulation of a phase stable, aqueous surfactant based antidandruff shampoo is difficult. In order to incorporate such effective, water insoluble antidandruff material into aqueous anionic surfactant-based hair shampoos, one or more suspending agents are required to keep the antidandruff agent homogeneously dispersed throughout the aqueous composition and to mitigate or eliminate the settling of the insoluble antidandruff material. Failure to adequately suspend the antidandruff material leads to eventual phase separation as the antidandruff material settles to the bottom of the container. Consequently, the consumer must vigorously shake the container of shampoo before each use to re-disperse the antidandruff active material. Manual shaking does not ensure that a homogeneous dispersion will be attained and the deposition of the active material to the hair scalp and skin during application and use may be uneven resulting in poor dandruff control and consumer dissatisfaction. In addition, there may also be aesthetic and sensory negatives to the user as uneven agglomerates of insoluble particles are deposited onto the hair.

The ideal suspending agent composition homogeneously disperses the antidandruff particles throughout the composition for an indefinite period of time without affecting the ideal viscosity, foaming, cleaning, or antidandruff properties of the shampoo. Many suspending agents operate on the principle of thickening the liquid to a great enough viscosity to retard the settling of particulate matter to such an extent that the product is stable over its lifetime. However, considering the relatively high percentage of antidandruff agent incorporated into antidandruff shampoos, a suspending agent relying only on thickening must be incorporated in such a high percentage to suspend the antidandruff agent that an unacceptably viscous product results. An increase in viscosity alone is not sufficient to afford permanent suspension of a dispersed phase. Stokes' law provides that merely increasing viscosity will delay but not stop separation or sedimentation of particles or droplets suspended in a liquid. This assumes of course that the particles are too large to be suspended by Brownian motion. Shampoos having too high a high viscosity are not acceptable to consumers since they are hard to dispense, hard to spread evenly on the hair and scalp, and often do not generate adequate foam. The ideal antidandruff shampoo should be thick enough to appear concentrated and rich and not run out of the container or hands too easily during application, and be thin enough for easy dispensing from the container, ease of application to the hair and even distribution over the scalp.

While a certain rheology modifier may thicken or enhance the viscosity of a composition in which it is included, it does not necessarily have desirable yield stress properties. A desirable yield stress property is critical to achieving certain physical and aesthetic characteristics in a liquid medium, such as the indefinite suspension of particles, insoluble liquid droplets, or the stabilization of gas bubbles within a liquid medium. Particles dispersed in a liquid medium will remain suspended if the yield stress (yield value) of the medium is sufficient to overcome the effect of gravity or buoyancy on those particles. Insoluble liquid droplets can be prevented from rising and coalescing and gas bubbles can be suspended and uniformly distributed in a liquid medium using yield value as a formulating tool. A yield stress polymer is used generally to adjust or modify the rheological properties of aqueous compositions. Such properties include, without limitation, viscosity improvement, flow rate improvement, stability to viscosity change over time, and the ability to suspend particles for indefinite periods of time.

Rheology modifiers have been used in shampoo products to increase viscosity at low shear rates and to maintain flow properties at higher shear rates. In addition, it has been discovered that certain rheology modifiers not only provide for a thickening effect, but also provide for effective storage stable suspensions of insoluble and particulate materials in aqueous surfactant systems. Acrylic polymers have been proposed for this purpose. U.S. Pat. No. 4,686,254 discloses a suspending agent for incompatible materials in water based systems. Incompatible materials include antidandruff agents such as zinc pyridinethione (zinc pyrithione). The suspending agent is a crosslinked copolymer prepared from (meth)acrylic acid and a C₁₀ to C₃₀ alkyl ester of (meth)acrylic acid.

U.S. Pat. No. 6,635,702 discloses a crosslinked acrylic emulsion polymer for use in aqueous surfactant containing compositions to thicken and stabilize products containing insoluble and particulate materials including insoluble particulate materials such as antidandruff agents. The compositions are said to be stable and have an attractive visual appearance.

U.S. Pat. No. 8,574,561 concerns an antidandruff shampoo composition containing an antidandruff agent such as zinc pyrithione, at least one viscosity modifying agent, at least one acrylic based polymeric compound different from the viscosity modifying agent, at least two surfactants chosen from amphoteric and zwitterionic surfactants, and optionally, a conditioning agent. The at least one viscosity modifying agent is defined as a carbomer, and the at least one acrylic polymer different from the viscosity modifying agent is defined as: 1) an acrylic copolymer prepared from two or more monomers consisting of (meth)acrylic acid or one of its simple esters, or 2) a copolymer prepared from the ester of methacrylic acid and the polyethylene glycol ether of a C₁₂ to C₂₂ fatty alcohol and one or more monomers of (meth)acrylic acid and one of its simple esters. A preferred at least one acrylic polymer different from the viscosity modifying agent is desirably crosslinked.

One approach for enhancing the efficacy of antidandruff compositions is to maximize the deposition of zinc pyrithione (ZPT) onto the scalp by using ZPT in combination with a secondary zinc salt. U.S. Pat. No. 8,491,877 discloses an aqueous surfactant containing antidandruff composition including ZPT (zinc pyridinethione) and a zinc layered material (ZLM) obtained from a zinc salt adjuvant material. Suitable ZLM's include hydrozincite (zinc carbonate hydroxide), basic zinc carbonate, aurichalcite (zinc copper carbonate hydroxide), and rosasite (copper zinc carbonate hydroxide) with a solubility of less than 25%. The formulator is not only challenged to provide for the efficacious suspension and dispersion of zinc pyridinethione within the formulation, equally challenging is the sparingly soluble zinc salt adjuvant material must be dispersed evenly throughout the composition so that it does not aggregate or settle.

An embodiment of the disclosure provides a stable composition for the ZLM dispersion where the ZLM zinc source exists in particulate form. It is revealed to be challenging to formulate aqueous systems containing a ZLM's, due to the compound's unique physical and chemical properties. The ZLM has a high density (approximately 3 g/cm³), and needs to be evenly dispersed throughout the composition so it will not aggregate or settle. The zinc-containing layered material also has a very-reactive surface chemistry as well as the propensity to dissolve in systems with pH values below 6.5. Accordingly, the pH of the composition is required to be greater than 6.5 in order to maintain an effective amount of zinc ions in the formulation to increase the bioavailability of ZPT to exert its antidandruff activity.

Currently used commercial rheology modifiers are pH-responsive microgels, viz., cross-linked polyacrylic acid polymers and alkali-swellable emulsion (ASE) polymers based on ethyl acrylate and methacrylic acid. Upon neutralization, these polymer beads swell to form a close-packed network of swollen particles providing the shampoo with yield stress, viscosity and shear-thinning. However, these pH-responsive microgels offer desired properties only within a limited span of pH and significant changes in properties are observed in the range of pH values close to the pKₐ (≈6.2) with significantly compromised yield-stress at pH above the pKₐ in shampoo systems. Besides, these anionically charged polymers are potent zinc chelating agents that reduce ZPT therapeutic efficacy. Therefore, without a properly designed system, anti-dandruff shampoo quality and performance can be negatively affected.

The disclosed crosslinked acrylic acid copolymers are viscosity building agents that increase the viscosity of compositions in which they are dissolved or dispersed upon suitable neutralization of the carboxylic acid moieties on the polymer backbone with an alkaline material. Indeed, viscosity allows for the controlled handling and dispensing of the product during use as compared to a thinner product. In personal care cleansing applications, a thick, rich shampoo or body cleanser is appealing to consumers from a sensory perspective. In addition, personal care cleansing products are expected to be easy to use. In other words, the shear thinning profile of the liquid composition should exhibit high viscosity at low shear conditions and lower viscosity at high shear conditions to aid in the application and removal of the product during use.

There are drawbacks associated with increasing the viscosity of a product beyond its ideal viscosity. Highly viscous products are typically difficult to apply and rinse away, especially if the shear thinning profile of the viscosity building agent is poor. High viscosities can also adversely affect packaging, dispensing, dissolution, and the foaming and sensory properties of the product.

While a certain rheology modifier may thicken or enhance the viscosity of a composition in which it is included, it does not necessarily have desirable yield stress properties. A desirable yield stress property is critical to achieving certain physical and aesthetic characteristics in a liquid medium, such as the indefinite suspension of particles, insoluble liquid droplets, or the stabilization of gas bubbles within a liquid medium. Particles dispersed in a liquid medium will remain suspended if the yield stress (yield value) of the medium is sufficient to overcome the effect of gravity or buoyancy on those particles. Insoluble liquid droplets can be prevented from rising and coalescing and gas bubbles can be suspended and uniformly distributed in a liquid medium using yield value as a formulating tool. A yield stress fluid is used generally to adjust or modify the rheological properties of aqueous compositions. Such properties include, without limitation, viscosity improvement, flow rate improvement, stability to viscosity change over time, and the ability to suspend particles for indefinite periods of time.

Despite the well-known benefits of utilizing crosslinked acrylic acid homopolymers and copolymers as a thickening, suspending, or rheology modifying agent, the wider use of such polymers have been limited by their incompatibility with formulations containing polyvalent cations, including, as discussed above, certain materials utilized as antidandruff materials, e.g., polyvalent metal salts of pyridinethione, such as zinc pyrithione.

The degradation and storage-instability of acrylic acid polymer thickened formulations containing sources of polyvalent cations has been observed in other compositions, including those containing calamine and zinc oxide. Historically, formulations thickened using these polymers and containing such ingredients have been stabilized where possible by initial adjustment to a pH greater than 8.5 to 9, thereby suppressing the hydrolysis and solubilization of the polyvalent cations. This approach, however, is untenable for most personal formulations designed for application to “delicate substrates” such as hair, scalp and skin.

Any antidandruff material in combination with a suspending agent added to a basic detersive surfactant chassis should provide antidandruff properties without detracting from the cleansing efficiency, aesthetic appeal and therapeutic efficacy of the composition in which they are contained. Unfortunately, antidandruff materials, particularly those containing polyvalent cations, in combination with polymeric suspending agents that contain anionic moieties often adversely affect physical properties (e.g. foaming ability, suspension stability and rheology profiles), as well as the therapeutic properties of the composition in which they are contained. There remains the challenge of formulating compositions which can effectively suspend insoluble antidandruff materials, particularly those containing polyvalent cations, such as zinc pyridinethione, while at the same time achieving good viscosity profiles, foam quality and suspension stability.

### SUMMARY

The disclosed technology relates to a composition containing in an aqueous medium:

a) at least one surfactant selected from an anionic, amphoteric, and zwitter ionic surfactant;
b) at least one antidandruff agent; and
c) a crosslinked nonionic, amphiphilic emulsion polymer;
wherein the emulsion polymer is prepared from a polymerizable monomer mixture comprising at least one hydrophilic monomer and at least one hydrophobic monomer, wherein said hydrophilic monomer is selected from hydroxy(C₁-C₅)alkyl (meth)acrylates, N-vinyl amides, amino group containing monomers, or mixtures thereof; wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof; and wherein the amphiphilic emulsion polymer is crosslinked with at least one amphiphilic crosslinking agent containing more than one unsaturated moiety.

It has been discovered that aqueous surfactant containing antidandruff hair care cleansing compositions possessing excellent phase stability and detersive properties are obtained by incorporating at least one crosslinked, nonionic, amphiphilic emulsion polymer into the formulation to provide stable antidandruff agent containing hair care cleansing compositions.

In one aspect, embodiments of the present technology relate to stable aqueous surfactant containing hair care cleansing compositions comprising an antidandruff agent and a conditioning agent that are stabilized by at least one crosslinked, nonionic, amphiphilic emulsion polymer.

In one aspect, embodiments of the disclosed technology relate to a aqueous surfactant containing hair care cleansing composition comprising an antidandruff agent, a silicone conditioning agent and a nonionic, amphiphilic emulsion polymer which provides stable suspensions of pearlescent and other insoluble materials to deliver an aesthetic appearance and good shelf appeal.

In one aspect, embodiments of the disclosed technology relate to a aqueous surfactant containing hair care cleansing composition comprising an antidandruff agent, a silicone conditioning agent and a crosslinked, nonionic, amphiphilic emulsion polymer which provides stable suspensions of pearlescent and other insoluble materials to deliver an aesthetic appearance and good shelf appeal over a wide range of pH values, affording more flexibility in the type of materials that can be formulated into the hair care composition as well as an extended range of yield stress properties not typically available with other polymeric thickeners.

In another aspect, an embodiment of the disclosed technology relates to a composition and method for improving the suspension stability of a thickened aqueous surfactant containing hair care composition comprising an antidandruff agent, at least one surfactant and at least one silicone conditioning agent, the composition and method comprising combining a crosslinked, nonionic amphiphilic emulsion polymer with at least one detersive surfactant selected from anionic surfactants, amphoteric surfactants, nonionic surfactants and combinations of two or more thereof, wherein the concentration of the amphiphilic emulsion polymer is no more than 5 wt. %, and the at least one surfactant is no more than 30 wt. % (all weight percentages are based on the total weight of the composition), wherein the yield stress of the composition is at least 0.1 Pa with a shear thinning index of less than 0.5 at shear rates between about 0.1 and about 1 reciprocal seconds, and wherein the yield stress, elastic modulus and optical clarity of the composition are substantially independent of pH ranging from about 2 to about 14.

In one aspect of the disclosed technology, the nonionic, amphiphilic emulsion polymer is prepared from a free radically polymerizable monomer composition comprising at least one hydrophilic monomer, at least one hydrophobic monomer, and at least one crosslinking agent.

In an embodiment, it has been found that amphiphilic crosslinking agents can be easily reacted into amphiphilic polymer. Amphiphilic crosslinking agents can contain more than one reactive moiety. In some embodiments the at least one reactive moiety can be an allyl group.

In one aspect of the disclosed technology, the hydrophilic monomer is selected from N-vinyl amides, amino(C₁-C₅)alkyl (meth)acrylates, hydroxy(C₁-C₅)alkyl (meth)acrylates, amino group containing monomers, or mixtures thereof. In one aspect, the hydrophobic monomer is selected from vinyl ester of an aliphatic carboxylic acid containing an acyl moiety having 2 to 22 carbon atoms, esters of (meth)acrylic acid with alcohols containing 1 to 30 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof. In one embodiment, the crosslinking monomer is selected from at least one polyunsaturated monomer containing at least two polymerizable unsaturated moieties.

In one aspect of the disclosed technology, the nonionic amphiphilic emulsion polymer is prepared from a free radically polymerizable monomer composition comprising at least one N-vinyl amide monomer, at least one vinyl ester of an aliphatic carboxylic acid containing an acyl moiety having 2 to 22 carbon atoms, and at least one crosslinking monomer, in optional combination with at least one monomer selected from esters of (meth)acrylic acid with alcohols containing 1 to 30 carbon atoms, associative monomers, semi-hydrophobic monomers, or mixtures thereof.

### DESCRIPTION OF EXEMPLARY EMBODIMENTS

Exemplary embodiments in accordance with the disclosed technology will be described. Various modifications, adaptations or variations of the exemplary embodiments described herein may become apparent to those skilled in the art as such are disclosed. It will be understood that all such modifications, adaptations or variations that rely upon the teachings of the disclosed technology, and through which these teachings have advanced the art, are considered to be within the scope and spirit of the presently disclosed technology.

The compositions, polymers and methods of the disclosed technology may suitably comprise, consist of, or consist essentially of the components, elements, steps, and process delineations described herein. The technology illustratively disclosed herein suitably may be practiced in the absence of any element which is not specifically disclosed herein.

Except as otherwise noted, the articles “a”, “an”, and “the” mean one or more.

Unless otherwise stated, all percentages, parts, and ratios expressed herein are based upon weight of the total compositions of the disclosed technology.

When referring to a specified monomer(s) that is incorporated into a polymer of the disclosed technology, it will be recognized that the monomer(s) will be incorporated into the polymer as a unit(s) derived from the specified monomer(s) (e.g., repeating unit).

As used herein, the term “amphiphilic polymer” means that the polymeric material has distinct hydrophilic and hydrophobic portions. “Hydrophilic” typically means a portion that interacts intramolecularly with water and other polar molecules. “Hydrophobic” typically means a portion that interacts preferentially with oils, fats or other non-polar molecules rather than aqueous media.

As used herein, the term “hydrophilic monomer” means a monomer that is substantially water soluble. “Substantially water soluble” refers to a material that is soluble in distilled (or equivalent) water, at 25° C., at a concentration of about 3.5% by weight in one aspect, and soluble at about 10% by weight in another aspect (calculated on a water plus monomer weight basis).

As used herein, the term “hydrophobic monomer” means a monomer that is substantially water insoluble. “Substantially water insoluble” refers to a material that is not soluble in distilled (or equivalent) water, at 25° C., at a concentration of about 3% by weight in one aspect, and not soluble at about 2.5% by weight in another aspect (calculated on a water plus monomer weight basis).

By “nonionic” is meant that a monomer, monomer composition or a polymer polymerized from a monomer composition is devoid of ionic or ionizable moieties (“nonionizable”).

An ionizable moiety is any group that can be made ionic by neutralization with an acid or a base.

An ionic or an ionized moiety is any moiety that has been neutralized by an acid or a base.

By “substantially nonionic” is meant that the monomer, monomer composition or polymer polymerized from a monomer composition contains less than 5 wt. % in one aspect, less than 3 wt. % in another aspect, less than 1 wt. % in a further aspect, less than 0.5 wt. % in a still further aspect, less than 0.1 wt. % in an additional aspect, and less than 0.05 wt. % in a further aspect, of an ionizable and/or an ionized moiety.

The prefix “(meth)acryl” includes “acryl” as well as “methacryl”. For example, the term (meth)acrylic includes both acrylic and methacrylic, and the term (meth)acrylate includes acrylate as well as methacrylate. By way of further example, the term “(meth)acrylamide” includes both acrylamide and methacrylamide.

The term “hair care composition” as used herein, without limitation, includes shampoos, soaps, body washes, shower gels and other aqueous surfactant containing formulations normally applied to the hair, scalp and skin.

Here, as well as elsewhere in the specification and claims, individual numerical values (including carbon atom numerical values), or limits, can be combined to form additional non-disclosed and/or non-stated ranges.

While overlapping weight ranges for the various components and ingredients that can be contained in the compositions of the disclosed technology have been expressed for selected embodiments and aspects of the technology, it should be readily apparent that the specific amount of each component in the disclosed compositions will be selected from its disclosed range such that the amount of each component is adjusted such that the sum of all components in the composition will total 100 weight percent. The amounts employed will vary with the purpose and character of the desired product and can be readily determined by one skilled in the art.

The headings provided herein serve to illustrate, but not to limit the disclosed technology in any way or manner.

### A. Amphiphilic Emulsion Polymer

The crosslinked, nonionic, amphiphilic polymers (for brevity crosslinked amphiphilic polymer) useful in the practice of the disclosed technology are polymerized from monomer components that contain free radically polymerizable unsaturation. In one embodiment, the crosslinked, nonionic, amphiphilic polymers useful in the practice of the disclosed technology are polymerized from a monomer composition comprising at least one nonionic, hydrophilic unsaturated monomer, at least one unsaturated hydrophobic monomer, and at least one amphiphilic polyunsaturated crosslinking monomer. In one aspect, the copolymer can be polymerized from a monomer composition comprising any weight ratio of nonionic, hydrophilic unsaturated monomer to unsaturated hydrophobic monomer.

In one embodiment, the copolymers can be prepared from a monomer composition typically having a hydrophilic monomer to hydrophobic monomer ratio of from about 5:95 wt. % to about 95:5 wt. % in one aspect, from about 15:85 wt. % to about 85:15 wt. % in another aspect, and from about 30:70 wt. % to about 70:30 wt. % in a further aspect, based on the total weight of the hydrophilic and hydrophobic monomers present. The hydrophilic monomer component can be selected from a single hydrophilic monomer or a mixture of hydrophilic monomers, and the hydrophobic monomer component can be selected from a single hydrophobic monomer or a mixture of hydrophobic monomers.

### Hydrophilic Monomer

The hydrophilic monomers suitable for the preparation of the crosslinked, nonionic, amphiphilic emulsion polymer compositions of the disclosed technology are selected from but are not limited to hydroxy(C₁-C₅)alkyl (meth)acrylates; open chain and cyclic N-vinylamides (N-vinyllactams containing 4 to 9 atoms in the lactam ring moiety, wherein the ring carbon atoms optionally can be substituted by one or more lower alkyl groups such as methyl, ethyl or propyl); amino group containing vinyl monomers selected from (meth)acrylamide, N—(C₁-C₅)alkyl(meth)acrylamides, N,N-di(C₁−C₅)alkyl(meth)acrylamides, N—(C₁-C₅)alkylamino(C₁-C₅)alkyl(meth)acrylamides and N,N-di(C₁-C₅)alkylamino(C₁-C₅)alkyl(meth)acrylamides, wherein the alkyl moieties on the disubstituted amino groups can be the same or different, and wherein the alkyl moieties on the monosubstituted and disubstituted amino groups can be optionally substituted with a hydroxyl group; other monomers include vinyl alcohol; vinyl imidazole; and (meth)acrylonitrile. Mixtures of the foregoing monomers also can be utilized.

The hydroxy(C₁-C₅)alkyl (meth)acrylates can be structurally represented by the following formula:

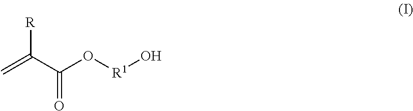

wherein R is hydrogen or methyl and R¹ is an divalent alkylene moiety containing 1 to 5 carbon atoms, wherein the alkylene moiety optionally can be substituted by one or more methyl groups. Representative monomers include 2-hydroxyethyl(meth)acrylate, 3-hydroxypropyl(meth)acrylate, 4-hydroxybutyl(meth)acrylate, and mixtures thereof.

Representative open chain N-vinylamides include N-vinylformamide, N-methyl-N-vinylformamide, N-(hydroxymethyl)-N-vinylformamide, N-vinylacetamide, N-vinylmethylacetamide, N-(hydroxymethyl)-N-vinylacetamide, and mixtures thereof.

Representative cyclic N-vinylamides (also known as N-vinyllactams) include N-vinyl-2-pyrrolidinone, N-(1-methyl vinyl) pyrrolidinone, N-vinyl-2-piperidone, N-vinyl-2-caprolactam, N-vinyl-5-methyl pyrrolidinone, N-vinyl-3,3-dimethyl pyrrolidinone, N-vinyl-5-ethyl pyrrolidinone and N-vinyl-6-methyl piperidone, and mixtures thereof. Additionally, monomers containing a pendant N-vinyl lactam moiety can also be employed, e.g., N-vinyl-2-ethyl-2-pyrrolidone (meth)acrylate.

The amino group containing vinyl monomers include (meth)acrylamide, diacetone acrylamide and monomers that are structurally represented by the following formulas:

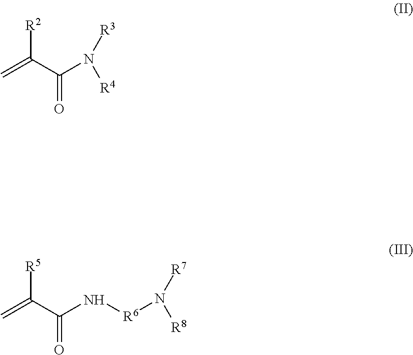

Formula (II) represents N—(C₁-C₅)alkyl(meth)acrylamide or N,N-di(C₁-C₅)alkyl(meth)acrylamide wherein R² is hydrogen or methyl, R³ independently is selected from hydrogen, C₁ to C₅ alkyl and C₁ to C₅ hydroxyalkyl, and R⁴ independently is selected from is C₁ to C₅ alkyl or C₁ to C₅ hydroxyalkyl.

Formula (III) represents N—(C₁-C₅)alkylamino(C₁-C₅)alkyl(meth)acrylamide or N,N-di(C₁-C₅)alkylamino(C₁-C₅)alkyl(meth)acrylamide wherein R⁵ is hydrogen or methyl, R⁶ is C₁ to C₅ alkylene, R⁷ independently is selected from hydrogen or C₁ to C₅ alkyl, and R⁸ independently is selected from C₁ to C₅ alkyl.

Representative N-alkyl(meth)acrylamides include but are not limited to N-methyl(meth)acrylamide, N-ethyl(meth)acrylamide, N-propyl(meth)acrylamide, N-isopropyl(meth)acrylamide, N-tert-butyl(meth)acrylamide, N-(2-hydroxyethyl)(meth)acrylamide, N-(3-hydroxypropyl)(meth)acrylamide, and mixtures thereof.

Representative N,N-dialkyl(meth)acrylamides include but are not limited to N,N-dimethyl(meth)acrylamide, N,N-diethyl(meth)acrylamide, N,N-(di-2-hydroxyethyl)(meth)acrylamide, N,N-(di-3-hydroxypropyl)(meth)acrylamide, N-methyl, N-ethyl(meth)acrylamide, and mixtures thereof.

Representative N,N-dialkylaminoalkyl(meth)acrylamides include but are not limited to N,N-dimethylaminoethyl(meth)acrylamide, N,N-diethylaminoethyl(meth)acrylamide, N,N-dimethylaminopropyl(meth)acrylamide, and mixtures thereof.

### Hydrophobic Monomer

Hydrophobic monomers suitable for the preparation of the crosslinked, nonionic, amphiphilic emulsion polymer compositions of the disclosed technology are selected from but are not limited to one or more of alkyl esters of (meth)acrylic acid having an alkyl group containing 1 to 30 carbon atoms; vinyl esters of aliphatic carboxylic acids containing 1 to 22 carbon atoms; vinyl ethers of alcohols containing 1 to 22 carbon atoms; vinyl aromatics containing 8 to 20 carbon atoms; vinyl halides; vinylidene halides; linear or branched alpha-monoolefins containing 2 to 8 carbon atoms; an associative monomer having a hydrophobic end group containing 8 to 30 carbon atoms, and mixtures thereof.

### Semi-Hydrophobic Monomer

Optionally, at least one alkoxylated semi-hydrophobic monomer can be used in the preparation of the amphiphilic emulsion polymers of the disclosed technology. A semi-hydrophobic monomer is similar in structure to an associative monomer, but has a substantially non-hydrophobic end group selected from hydroxyl or a moiety containing 1 to 4 carbon atoms.

In one aspect, of the disclosed technology, alkyl esters of (meth)acrylic acid having an alkyl group containing 1 to 22 carbon atoms can be represented by the following formula:

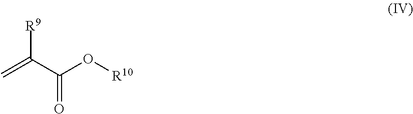

wherein R⁹ is hydrogen or methyl and R¹⁰ is C₁ to C₂₂ alkyl

Representative monomers under formula (IV) include but are not limited to methyl (meth)acrylate, ethyl (meth)acrylate, butyl (meth)acrylate, sec-butyl (meth)acrylate, iso-butyl (meth)acrylate, hexyl (meth)acrylate), heptyl (meth)acrylate, octyl (meth)acrylate, 2-ethylhexyl (meth)acrylate, decyl (meth)acrylate, isodecyl (meth)acrylate, lauryl (meth)acrylate, tetradecyl (meth)acrylate, hexadecyl (meth)acrylate, stearyl (meth)acrylate, behenyl (meth)acrylate, and mixtures thereof.

Vinyl esters of aliphatic carboxylic acids containing 1 to 22 carbon atoms can be represented by the following formula:

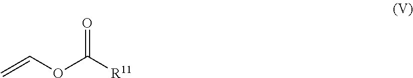

wherein R¹¹ is a C₁ to C₂₂ aliphatic group which can be an alkyl or alkenyl. Representative monomers under formula (V) include but are not limited to vinyl acetate, vinyl propionate, vinyl butyrate, vinyl isobutyrate, vinyl valerate, vinyl hexanoate, vinyl 2-methylhexanoate, vinyl 2-ethylhexanoate, vinyl iso-octanoate, vinyl nonanoate, vinyl neodecanoate, vinyl decanoate, vinyl versatate, vinyl laurate, vinyl palmitate, vinyl stearate, and mixtures thereof.

In one aspect, the vinyl ethers of alcohols containing 1 to 22 carbon atoms can be represented by the following formula:

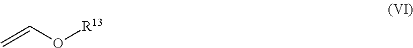

wherein R¹³ is a C₁ to C₂₂ alkyl. Representative monomers of formula (VI) include methyl vinyl ether, ethyl vinyl ether, butyl vinyl ether, isobutyl vinyl ether, 2-ethylhexyl vinyl ether, decyl vinyl ether, lauryl vinyl ether, stearyl vinyl ether, behenyl vinyl ether, and mixtures thereof.

Representative vinyl aromatic monomers include but are not limited to styrene, alpha-methylstyrene, 3-methyl styrene, 4-methyl styrene, 4-propyl styrene, 4-tert-butyl styrene, 4-n-butyl styrene, 4-n-decyl styrene, vinyl naphthalene, and mixtures thereof.

Representative vinyl and vinylidene halides include but are not limited to vinyl chloride and vinylidene chloride, and mixtures thereof.

Representative alpha-olefins include but are not limited to ethylene, propylene, 1-butene, iso-butylene, 1-hexene, and mixtures thereof.

The associative monomer of the disclosed technology has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer, and a hydrophobic end group portion (iii) for providing selective hydrophobic properties to the polymer.

The portion (i) supplying the ethylenically unsaturated end group can be a residue derived from an α,β-ethylenically unsaturated monocarboxylic acid. Alternatively, portion (i) of the associative monomer can be a residue derived from an allyl ether or vinyl ether; a nonionic vinyl-substituted urethane monomer, such as disclosed in U.S. Reissue Pat. No. 33,156 or U.S. Pat. No. 5,294,692; or a vinyl-substituted urea reaction product, such as disclosed in U.S. Pat. No. 5,011,978; the relevant disclosures of each are incorporated herein by reference.

The mid-section portion (ii) is a polyoxyalkylene segment of about 2 to about 150 in one aspect, from about 10 to about 120 in another aspect, and from about 15 to about 60 in a further aspect of repeating C₂-C₄ alkylene oxide units. The mid-section portion (ii) includes polyoxyethylene, polyoxypropylene, and polyoxybutylene segments, and combinations thereof comprising from about 2 to about 150 in one aspect, from about 5 to about 120 in another aspect, from about 10 to about 60 in a further aspect, and from about 15 to about 30 in a still further aspect of ethylene, propylene and/or butylene oxide units, arranged in random or block sequences of ethylene oxide, propylene oxide and/or butylene oxide units.

The hydrophobic end group portion (iii) of the associative monomer is a hydrocarbon moiety belonging to one of the following hydrocarbon classes: a C₈-C₃₀ linear alkyl, a C₈-C₃₀ branched alkyl, a C₂-C₃₀ alkyl-substituted phenyl, aryl-substituted C₂-C₃₀ alkyl groups, a C₇-C₃₀ saturated or unsaturated carbocyclic alkyl group. The saturated or unsaturated carbocyclic moiety can be a C₁-C₅ alkyl substituted or unsubstituted monocyclic or bicyclic moiety. In one aspect the bicyclic moiety is selected from bicycloheptyl or bicycloheptenyl. In another aspect the bicycloheptenyl moiety is disubstituted with the alkyl substituent(s). In a further aspect the bicycloheptenyl moiety is disubstituted with methyl on the same carbon atom.

Non-limiting examples of suitable hydrophobic end group portions (iii) of the associative monomers are linear or branched alkyl groups having about 8 to about 30 carbon atoms, such as capryl (C₈), iso-octyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and the like.

Examples of linear and branched alkyl groups having about 8 to about 30 carbon atoms that are derived from a natural source include, without being limited thereto, alkyl groups derived from hydrogenated peanut oil, soybean oil and canola oil (all predominately C₁₈), hydrogenated tallow oil (C₁₆-C₁₈), and the like; and hydrogenated C₁₀-C₃₀ terpenols, such as hydrogenated geraniol (branched C₁₀), hydrogenated farnesol (branched Cis), hydrogenated phytol (branched C₂₀), and the like.

Non-limiting examples of suitable C₂-C₃₀ alkyl-substituted phenyl groups include octylphenyl, nonylphenyl, decylphenyl, dodecylphenyl, hexadecylphenyl, octadecylphenyl, isooctylphenyl, sec-butylphenyl, and the like.

Exemplary aryl-substituted C₂-C₄₀ alkyl groups include, without limitation, styryl (e.g., 2-phenylethyl), distyryl (e.g., 2,4-diphenylbutyl), tristyryl (e.g., 2,4,6-triphenylhexyl), 4-phenylbutyl, 2-methyl-2-phenylethyl, tristyrylphenolyl, and the like.

Suitable C₇-C₃₀ carbocyclic groups include, without limitation, groups derived from sterols from animal sources, such as cholesterol, lanosterol, 7-dehydrocholesterol, and the like; from vegetable sources, such as phytosterol, stigmasterol, campesterol, and the like; and from yeast sources, such as ergosterol, mycosterol, and the like. Other carbocyclic alkyl hydrophobic end groups useful in the disclosed technology include, without limitation, cyclooctyl, cyclododecyl, adamantyl, decahydronaphthyl, and groups derived from natural carbocyclic materials, such as pinene, hydrogenated retinol, camphor, isobornyl alcohol, norbornyl alcohol, nopol and the like.

Useful associative monomers can be prepared by any method known in the art. See, for example, U.S. Pat. No. 4,421,902 to Chang et al.; U.S. Pat. No. 4,384,096 to Sonnabend; U.S. Pat. No. 4,514,552 to Shay et al.; U.S. Pat. No. 4,600,761 to Ruffner et al.; U.S. Pat. No. 4,616,074 to Ruffner; U.S. Pat. No. 5,294,692 to Barron et al.; U.S. Pat. No. 5,292,843 to Jenkins et al.; U.S. Pat. No. 5,770,760 to Robinson; U.S. Pat. No. 5,412,142 to Wilkerson, III et al.; and U.S. Pat. No. 7,772,421, to Yang et al., the pertinent disclosures of which are incorporated herein by reference.

In one aspect, exemplary associative monomers include those represented by formulas (VII) and (VIIA) as follows:

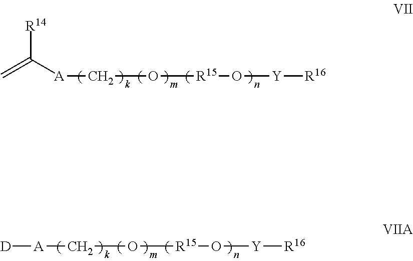

wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150 in one aspect, from about 10 to about 120 in another aspect, and from about 15 to about 60 in a further aspect; Y is —R¹⁵O—, —R¹⁵NH—, —C(O)—, —C(O)NH—, —R¹⁵NHC(O)NH—, —C(O)NHC(O)—, or a divalent alkylene radical containing 1 to 5 carbon atoms, e.g., methylene, ethylene, propylene, butylene, pentylene; R¹⁶ is a substituted or unsubstituted alkyl selected from a C₈-C₃₀ linear alkyl, a C₈-C₃₀ branched alkyl, a C₇-C₃₀ carbocyclic, a C₂-C₃₀ alkyl-substituted phenyl, an araalkyl substituted phenyl, and an aryl-substituted C₂-C₃₀ alkyl; wherein the R¹⁶ alkyl group, aryl group, phenyl group, or carbocyclic group optionally comprises one or more substituents selected from the group consisting of a methyl group, a hydroxyl group, an alkoxyl group, benzyl group phenylethyl group, and a halogen group. In one aspect, Y is ethylene and R¹⁶ is

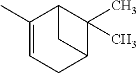

In one aspect, the hydrophobically modified associative monomer is an alkoxylated (meth)acrylate having a hydrophobic group containing 8 to 30 carbon atoms represented by the following Formula VB as follows:

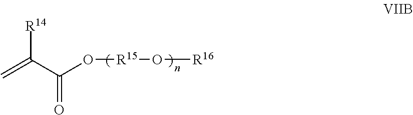

wherein R¹⁴ is hydrogen or methyl; R¹⁵ is a divalent alkylene moiety independently selected from C₂H₄, O₃H₆, and C₄H₈, and n represents an integer ranging from about 2 to about 150 in one aspect, from about 5 to about 120 in another aspect, from about 10 to about 60 in a further aspect, and from about 15 to about 30 in a still further aspect, (R¹⁵—O) can be arranged in a random or a block configuration; R¹⁶ is a substituted or unsubstituted alkyl selected from a C₈-C₃₀ linear alkyl, a C₈-C₃₀ branched alkyl, an alkyl substituted and unsubstituted C₇-C₃₀ carbocyclic alkyl, a C₂-C₃₀ alkyl-substituted phenyl, and an aryl-substituted C₂-C₃₀ alkyl.

Representative monomers under Formula V include lauryl polyethoxylated (meth)acrylate (LEM), cetyl polyethoxylated (meth)acrylate (CEM), cetearyl polyethoxylated (meth)acrylate (CSEM), stearyl polyethoxylated (meth)acrylate, arachidyl polyethoxylated (meth)acrylate, behenyl polyethoxylated (meth)acrylate (BEM), cerotyl polyethoxylated (meth)acrylate, montanyl polyethoxylated (meth)acrylate, melissyl polyethoxylated (meth)acrylate, phenyl polyethoxylated (meth)acrylate, nonylphenyl polyethoxylated (meth)acrylate, ω-tristyrylphenyl polyoxyethylene (meth)acrylate, where the polyethoxylated portion of the monomer contains about 2 to about 150 ethylene oxide units in one aspect, from about 5 to about 120 in another aspect, from about 10 to about 60 in a further aspect and from about 15 to about 30 in a still further aspect; octyloxy polyethyleneglycol (8) polypropyleneglycol (6) (meth)acrylate, phenoxy polyethylene glycol (6) polypropylene glycol (6) (meth)acrylate, and nonylphenoxy polyethylene glycol polypropylene glycol (meth)acrylate.

The semi-hydrophobic monomers of the disclosed technology are structurally similar to the associative monomer described above, but have a substantially non-hydrophobic end group portion. The semi-hydrophobic monomer has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer and a semi-hydrophobic end group portion (iii). The unsaturated end group portion (i) supplying the vinyl or other ethylenically unsaturated end group for addition polymerization is preferably derived from an α,β-ethylenically unsaturated mono carboxylic acid. Alternatively, the end group portion (i) can be derived from an allyl ether residue, a vinyl ether residue or a residue of a nonionic urethane monomer.

The polyoxyalkylene mid-section (ii) specifically comprises a polyoxyalkylene segment, which is substantially similar to the polyoxyalkylene portion of the associative monomers described above. In one aspect, the polyoxyalkylene portions (ii) include polyoxyethylene, polyoxypropylene, and/or polyoxybutylene units comprising from about 2 to about 150 in one aspect, from about 5 to about 120 in another aspect, from about 10 to about 60, and from about 15 to about 30 in a still further aspect in a further aspect of ethylene oxide, propylene oxide, and/or butylene oxide units, arranged in random or blocky sequences.

In one aspect, the semi-hydrophobic monomer can be represented by the following formulas:

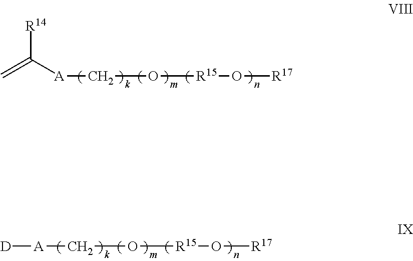

wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, O₃H₆, or C₄H₈, and combinations thereof; and n is an integer in the range of about 2 to about 150 in one aspect, from about 5 to about 120 in another aspect, and from about 10 to about 60, and from about 15 to about 30 in a still further aspect in a further aspect; R¹⁷ is selected from hydrogen and a linear or branched C₁-C₄ alkyl group (e.g., methyl, ethyl, propyl, iso-propyl, butyl, iso-butyl, and tert-butyl); and D represents a vinyl or an allyl moiety.

In one aspect, the semi-hydrophobic monomer under formula VIII can be represented by the following formulas:

CH₂═C(R¹⁴)C(O)O—(C₂H₄O)ₐ(C₃H₆O)_{b}—H VIIIA

CH₂═C(R¹⁴)C(O)O—(C₂H₄O)ₐ(C₃H₆O)_{b}—CH₃ VIIIB

wherein R¹⁴ is hydrogen or methyl, and “a” is an integer ranging from 0 or 2 to about 120 in one aspect, from about 5 to about 45 in another aspect, and from about 10 to about 0.25 in a further aspect, and “b” is an integer ranging from about 0 or 2 to about 120 in one aspect, from about 5 to about 45 in another aspect, and from about 10 to about 25 in a further aspect, subject to the proviso that “a” and “b” cannot be 0 at the same time.

Examples of semi-hydrophobic monomers under formula VIIIA include polyethyleneglycol methacrylate available under the product names Blemmer® PE-90 (R¹⁴=methyl, a=2, b=0), PE-200 (R¹⁴=methyl, a=4.5, b=0), and PE-350 (R¹⁴=methyl a=8, b=0); polypropylene glycol methacrylate available under the product names Blemmer® PP-1000 (R¹⁴=methyl, b=4-6, a=0), PP-500 (R¹⁴=methyl, a=0, b=9), PP-800 (R¹⁴=methyl, a=0, b=13); polyethyleneglycol polypropylene glycol methacrylate available under the product names Blemmer® 50PEP-300 (R¹⁴=methyl, a=3.5, b=2.5), 70PEP-350B (R¹⁴=methyl, a=5, b=2); polyethyleneglycol acrylate available under the product names Blemmer® AE-90 (R¹⁴=hydrogen, a=2, b=0), AE-200 (R¹⁴=hydrogen, a=2, b=4.5), AE-400 (R¹⁴=hydrogen, a=10, b=0); polypropyleneglycol acrylate available under the product names Blemmer® AP-150 (R¹⁴=hydrogen, a=0, b=3), AP-400(R¹⁴=hydrogen, a=0, b=6), AP-550 (R¹⁴=hydrogen, a=0, b=9). Blemmer® is a trademark of NOF Corporation, Tokyo, Japan.

Examples of semi-hydrophobic monomers under formula VIIIB include methoxypolyethyleneglycol methacrylate available under the product names Visiomer® MPEG 750 MA W (R¹⁴=methyl, a=17, b=0), MPEG 1005 MA W (R¹⁴=methyl, a=22, b=0), MPEG 2005 MA W (R¹⁴=methyl, a=45, b=0), and MPEG 5005 MA W (R¹⁴=methyl, a=113, b=0) from Evonik Rohm GmbH, Darmstadt, Germany); Bisomer® MPEG 350 MA (R¹⁴=methyl, a=8, b=0), and MPEG 550 MA (R¹⁴=methyl, a=12, b=0) from GEO Specialty Chemicals, Ambler Pa.; Blemmer® PME-100 (R¹⁴=methyl, a=2, b=0), PME-200 (R¹⁴=methyl, a=4, b=0), PME-400 (R¹⁴=methyl, a=9, b=0), PME-1000 (R¹⁴=methyl, a=23, b=0), PME-4000 (R¹⁴=methyl, a=90, b=0).

In one aspect, the semi-hydrophobic monomer set forth in formula IX can be represented by the following formulas:

CH₂═CH—O—(CH₂)_{d}—O—(C₃H₆O)ₑ—(C₂H₄O)_{f}—H IXA

CH₂═CH—CH₂—O—(C₃H₆O)_{g}—(C₂H₄O)ₕ—H IXB

wherein d is an integer of 2, 3, or 4; e is an integer in the range of from about 1 to about 10 in one aspect, from about 2 to about 8 in another aspect, and from about 3 to about 7 in a further aspect; f is an integer in the range of from about 5 to about 50 in one aspect, from about 8 to about 40 in another aspect, and from about 10 to about 30 in a further aspect; g is an integer in the range of from 1 to about 10 in one aspect, from about 2 to about 8 in another aspect, and from about 3 to about 7 in a further aspect; and h is an integer in the range of from about 5 to about 50 in one aspect, and from about 8 to about 40 in another aspect; e, f, g, and h can be 0 subject to the proviso that e and f cannot be 0 at the same time, and g and h cannot be 0 at the same time.

Monomers under formulas IXA and IXB are commercially available under the trade names Emulsogen® R109, R208, R307, RAL109, RAL208, and RAL307 sold by Clariant Corporation; BX-AA-E5P5 sold by Bimax, Inc.; and combinations thereof. EMULSOGEN7 R109 is a randomly ethoxylated/propoxylated 1,4-butanediol vinyl ether having the empirical formula CH₂═CH—O(CH₂)₄₀(C₃H₆O)₄(C₂H₄O)₁₀H; Emulsogen® R208 is a randomly ethoxylated/propoxylated 1,4-butanediol vinyl ether having the empirical formula CH₂═CH—O(CH₂)₄₀(C₃H₆O)₄(C₂H₄O)₂₀H; Emulsogen® R307 is a randomly ethoxylated/propoxylated 1,4-butanediol vinyl ether having the empirical formula CH₂═CH—O(CH₂)₄₀(C₃H₆O)₄(C₂H₄O)₃₀H; Emulsogen® RAL109 is a randomly ethoxylated/propoxylated allyl ether having the empirical formula CH₂═CHCH₂O(C₃H₆O)₄(C₂H₄O)₁₀H; Emulsogen® RAL208 is a randomly ethoxylated/propoxylated allyl ether having the empirical formula CH₂═CHCH₂O(C₃H₆O)₄(C₂H₄O)₂₀H; Emulsogen® RAL307 is a randomly ethoxylated/propoxylated allyl ether having the empirical formula CH₂═CHCH₂O(C₃H₆O)₄(C₂H₄O)₃₀H; and BX-AA-E5P5 is a randomly ethoxylated/propoxylated allyl ether having the empirical formula CH₂═CHCH₂O(C₃H₆O)₅(C₂H₄O)₅H.

In the associative and the semi-hydrophobic monomers of the disclosed technology, the polyoxyalkylene mid-section portion contained in these monomers can be utilized to tailor the hydrophilicity and/or hydrophobicity of the polymers in which they are included. For example, mid-section portions rich in ethylene oxide moieties are more hydrophilic while mid-section portions rich in propylene oxide moieties are more hydrophobic. By adjusting the relative amounts of ethylene oxide to propylene oxide moieties present in these monomers the hydrophilic and hydrophobic properties of the polymers in which these monomers are included can be tailored as desired.

The amount of associative and/or semi-hydrophobic monomer utilized in the preparation of the polymers of the present disclosed technology can vary widely and depends, among other things, on the final rheological and aesthetic properties desired in the polymer. When utilized, the monomer reaction mixture contains one or more monomers selected from the associative and/or semi-hydrophobic monomers disclosed above in amounts ranging from about 0.01 to about 15 wt. % in one aspect, from about 0.1 wt. % to about 10 wt. % in another aspect, from about 0.5 to about 8 wt. % in still another aspect and from about 1, 2 or 3 to about 5 wt. % in a further aspect, based on the weight of the total monomers.

### Ionizable Monomer

In one aspect of the disclosed technology, the nonionic, amphiphilic emulsion polymer compositions can be polymerized from a monomer composition including 0 to 5 wt. % of an ionizable and/or ionized monomer, based on the weight of the total monomers, so long as the mitigation of silicone deposition loss and/or the yield stress value of the surfactant compositions in which the polymers of the disclosed technology are included are not deleteriously affected.

In another aspect, the amphiphilic emulsion polymer compositions of the disclosed technology can be polymerized from a monomer composition comprising less than 3 wt. % in one aspect, less than 1 wt. % in a further aspect, less than 0.5 wt. % in a still further aspect, less than 0.1 wt. % in an additional aspect, and less than 0.05 wt. % in a further aspect, of an ionizable and/or an ionized moiety, based on the weight of the total monomers.

Ionizable monomers include monomers having a base neutralizable moiety and monomers having an acid neutralizable moiety. Base neutralizable monomers include olefinically unsaturated monocarboxylic and dicarboxylic acids and their salts containing 3 to 5 carbon atoms and anhydrides thereof. Examples include (meth)acrylic acid, itaconic acid, maleic acid, maleic anhydride, and combinations thereof. Other acidic monomers include styrenesulfonic acid, acrylamidomethylpropanesulfonic acid (AMPS® monomer), vinylsulfonic acid, vinylphosphonic acid, allylsulfonic acid, methallylsulfonic acid; and salts thereof.

Acid neutralizable monomers include olefinically unsaturated monomers which contain a basic nitrogen atom capable of forming a salt or a quaternized moiety upon the addition of an acid. For example, these monomers include vinylpyridine, vinylpiperidine, vinylimidazole, vinylmethylimidazole, dimethylaminomethyl (meth)acrylate, dimethylaminoethyl (meth)acrylate, diethylaminomethyl (meth)acrylate and methacrylate, dimethylaminoneopentyl (meth)acrylate, dimethylaminopropyl (meth)acrylate, and diethylaminoethyl (meth)acrylate.

### Crosslinking Monomer

In one embodiment, the crosslinked, nonionic, amphiphilic polymers useful in the practice of the disclosed technology are polymerized from a monomer composition comprising a first monomer comprising at least one nonionic, hydrophilic unsaturated monomer, at least one nonionic, unsaturated hydrophobic monomer, and mixtures thereof, and a third monomer comprising at least one polyunsaturated crosslinking monomer. The crosslinking monomer(s) is utilized to polymerize covalent crosslinks into the polymer backbone. The crosslinking monomer can be an amphiphilic crosslinking agent or a mixture of an amphiphilic crosslinking agent and a conventional crosslinking agent.

The crosslinking monomer can be an amphiphilic crosslinking agent. The amphiphilic crosslinking agent is utilized to polymerize covalent crosslinks into the amphiphilic polymer backbone. In some instances, conventional crosslinking agents can affect the volume expansion or swelling of micro-gel particles in fluids containing surfactants. For example, a high level of conventional crosslinking agent could provide a high yield stress but the limited expansion of the micro-gels would result in undesirably high polymer use levels and low optical clarity. On the other hand, a low level of conventional crosslinking agents could give high optical clarity but low yield stress. It is desirable that polymeric micro-gels allow maximum swelling while maintaining a desirable yield stress, and it has been found that the use of amphiphilic crosslinking agents in place of, or in conjunction with conventional crosslinking agents can provide just these benefits. In addition, it has been found that the amphiphilic crosslinking agent can be easily reacted into the amphiphilic polymer. Often, certain processing techniques, such as staging, can be required with conventional crosslinking agents to achieve the proper balance of optical clarity and yield stress. In contrast, it has been found that amphiphilic crosslinking agents can simply be added in a single stage with the monomer mixture.

Amphiphilic crosslinking agents are a subset of compounds known in the art as reactive surfactants. Reactive surfactants are surface acting agents containing at least one reactive moiety so that they can covalently link to the surface of polymeric particles. By linking to particles, the reactive surfactants can improve the colloidal stability of latex particles due to the surfactant's resistance to desorbing from the particle surface. Reactive surfactants in the art commonly only have, or only need, one reactive moiety to prevent such desorption.

As a subset of reactive surfactants, amphiphilic crosslinking agents as used herein are those compounds or mixtures thereof that include more than one reactive moiety. It has surprisingly been found that such amphiphilic crosslinking agents not only can be employed to improve stability of particles, but can be efficiently employed to prepare yield stress fluids as described herein.

The art is replete with disclosures on various types of reactive surfactants, and one of ordinary skill in the art could readily determine which include more than one reactive moiety such that they can be employed herein as amphiphilic crosslinking agents without undue experimentation. Exemplary non-limiting amphiphilic crosslinking agents can be seen, for example, in U.S. Pat. No. 3,541,138 (issued Nov. 17, 1970 to Emmons et al.), U.S. Pat. No. 6,262,152 (issued Jul. 17, 2001 to Fryd et al.), U.S. Pat. No. 8,354,488 (issued Jan. 15, 2013 to Li et al.), WO2002/100525 (published Dec. 19, 2002 to Syngenta), and the like.

The amphiphilic crosslinking agent contains a hydrophobic moiety and a hydrophilic moiety. The hydrophobic moiety will provide solubility in oils, and the hydrophilic moiety will provide water solubility. Hydrophobic and hydrophilic moieties are well known to those skilled in the art.

Non-limiting examples of hydrophobic moieties of the amphiphilic crosslinking agent can be derived from functional groups such as alkyl, aryl, and alkyl aryl acrylates or methacrylates having 1-12 carbon atoms in the alkyl group and/or 6-12 carbons in the aryl group such as methyl, ethyl, butyl, propyl, isobutyl, hexyl, 2-ethyl hexyl, nonyl, lauryl, isobornyl, benzyl acrylates and methacrylates and the like; polymerizable vinyl aromatic monomers such as styrene, alpha methyl styrene, vinyl toluene and the like; and aliphatic hydrocarbon monomers such as isoprene and butadiene. Regardless of the constituent functional groups from which the hydrophobic moiety of the amphiphilic crosslinking agent is derived, the hydrophobic moiety will have limited solubility in water, which one of ordinary skill in the art would readily envisage. Examples of functional groups for preparing hydrophobic moieties can include, for example, alkyl phenols, stearyls, lauryls, tri-styryl phenols, groups derived from natural oils, and the like.

Non-limiting examples of hydrophilic moieties of the amphiphilic crosslinking agent can be functional groups such as ethoxylates, hydroxyls, amidos, aminos, phosphates, phosponates, sulfates, sulfonates, carboxylates and the like. Such hydrophilic moieties of the amphiphilic crosslinking agent can be derived from, for example, acid monomers such as acrylic acid, methacrylic acid, acrylamidomethylpropane sulfonic acid, itaconic acid, maleic acid and styrene sulfonic acid and esters thereof; amine-containing monomers such as 2-dimethylaminoethyl methacrylate, 2-dimethylaminoethyl acrylate, 2-diethylaminoethyl methacrylate, and 2-diethylaminoethyl acrylate; and monomers having oligoether moieties of the general formula:

CH₂═C(R)C(O)O(C₂H₄O)ₙR₁

wherein R═H or methyl; R₁=alkyl of 1 to 4 carbon atoms, aryl of 6 to 12 carbon atoms, or alkyl-aryl, and n=1 to 20, examples of which include ethoxyethyl methacrylate, butoxyethyl methacrylate, ethoxytriethylene methacrylate, methoxy-polyethylene glycol methacrylate, and 2-ethoxytriethylene glycol methacrylate.

Additionally, the amphiphilic crosslinking agent contains multiple crosslinking moieties. Non-limiting examples of crosslinking moieties can include those shown in Table A below.

TABLE A

| Functional Group | Crosslinking Group |
|---|---|
| epoxy | amine, anhydride, acid, phenolic, hydroxyl, |
| | N-methylol, aldehyde, acetoacetoxy |
| hydroxyl | isocyanate, epoxide, N-methylol, anhydride, ester |
| anhydride | epoxide, amine, hydroxyl |
| cyclic carbonate | Amine |
| activated allyl | amine, radical inhibitor |
| amine | aldehyde, epoxide, anhydride, isocyanate, ester, |
| | acetoacetoxy, activated allyl |
| silane, silicate, | hydroxyl, water, acid, isocyanate, silane, silicate |
| silanol, silicone | |
| imine | aldehyde, epoxide, anhydride, isocyanate, ester, |
| | acetoacetoxy, activated allyl |

Further non-limiting examples of crosslinking moieties can include unsaturated moieties. In an embodiment, the amphiphilic crosslinking agent contains more than one unsaturated moiety, or at least two unsaturated moieties. In one aspect, the amphiphilic crosslinking agent is a polyunsaturated compound containing at least 2 unsaturated moieties. In another aspect, the amphiphilic crosslinking agent contains at least 3 unsaturated moieties.

Mixtures of two or more amphiphilic crosslinking agents can also be utilized to crosslink the nonionic, amphiphilic polymers. In one aspect, the mixture of amphiphilic crosslinking agent contains more than one unsaturated moieties, or an average of 1.5 or 2 unsaturated moieties. In another aspect, the mixture of amphiphilic crosslinking agents contains an average of 2.5 unsaturated moieties. In still another aspect, the mixture of amphiphilic crosslinking agents contains an average of about 3 unsaturated moieties. In a further aspect, the mixture of amphiphilic crosslinking agents contains an average of about 3.5 unsaturated moieties.

In one aspect, exemplary amphiphilic crosslinking agents suitable for use with the present technology can include, but not be limited to, compounds such as those disclosed in US 2013/0047892 (published Feb. 28, 2013 to Palmer, Jr. et al.), represented by the following formulas:

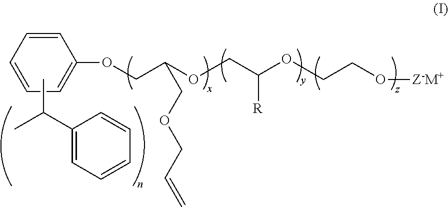

where R═CH₃, CH₂CH₃, C₆H₅, or C₁₄H₂₉; n=1, 2, or 3; x is 2-10, y is 0-200, z is 4-200, more preferably from about 5 to 60, and most preferably from about 5 to 40; Z can be either SO₃⁻ or PO₃²⁻, and M⁺ is Na⁺, K⁺, NH₄⁺, or an alkanolamine such as, for example, monoethanolamine, diethanolamine, and triethanolamine;

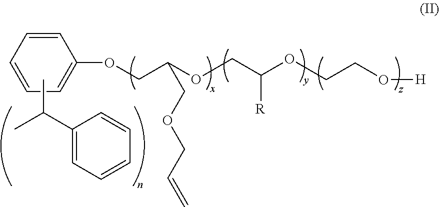

where R═CH₃, CH₂CH₃, C₆H₅, or C₁₄H₂₉; n=1, 2, 3; x is 2-10, y is 0-200, z is 4-200, more preferably from about 5 to 60, and most preferably from about 5 to 40;

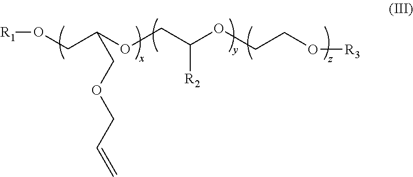

where R₁ is a C₁₀₋₂₄ alkyl, alkaryl, alkenyl, or cycloalkyl, R₂═CH₃, CH₂CH₃, C₆H₅, or C₁₄H₂₉; x is 2-10, y is 0-200, z is 4-200, more preferably from about 5 to 60, and most preferably from about 5 to 40; and R₃ is H or Z⁻ M⁺ Z can be either SO₃⁻ or PO₃²⁻, and M⁺ is Na⁺, K⁺, NH₄⁺, or an alkanolamine such as, for example, monoethanolamine, diethanolamine, and triethanolamine.

The foregoing amphiphilic crosslinking agents conforming to formulas (I), (11), (III), (IV) and (V) are disclosed in U.S. Patent Application Publication No. US 2014/0114006, the disclosure of which is herein incorporated by reference, and are commercially available under the E-Sperse™ RS Series trade name (e.g., product designations RS-1617, RS-1618, RS-1684) from Ethox Chemicals, LLC.

In one embodiment, the amphiphilic crosslinking agent can be used in an amount ranging from about 0.01 to about 3 wt. % in one aspect, from about 0.05 to about 0.1 wt. % in another aspect, and from about 0.1 to about 0.75 wt. % in a further aspect, based on the dry weight of the nonionic, amphiphilic polymer of the disclosed technology.

In another embodiment, the amphiphilic crosslinking agent can contain an average of about 1.5 or 2 unsaturated moieties and can be used in an amount ranging from about 0.01 to about 3 wt. % in one aspect, from about 0.02 to about 1 wt. % in another aspect, from about 0.05 to about 0.75 wt. % in a further aspect, and from about 0.075 to about 0.5 wt. % in a still further aspect, and from about 0.1 to about 0.15 wt. % in another aspect, based upon the total weight of the, nonionic, amphiphilic polymer of the disclosed technology.

In one aspect, the amphiphilic crosslinking agent is selected from compounds of formulas (III), (IV) or (V).

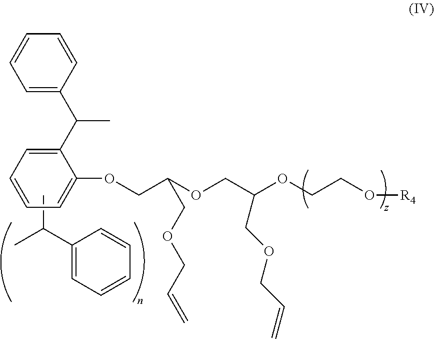

where n is 1 or 2; z is 4 to 40 in one aspect, 5 to 38 in another aspect, and 10 to 20 in a further aspect; and R₄ is H, SO₃⁻M⁺ or PO₃⁻M⁺, and M is selected from Na, K, and NH₄.

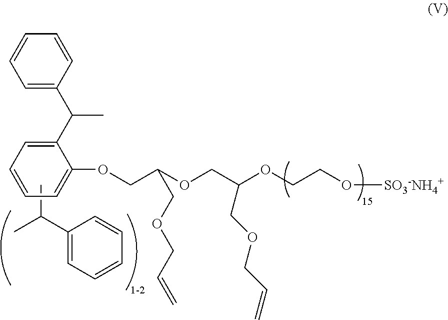

In one embodiment, the crosslinking monomer can include a combination of an amphiphilic crosslinking agent and a conventional crosslinking agent. In one aspect, the conventional crosslinking agent is a polyunsaturated compound containing at least 2 unsaturated moieties. In another aspect, the conventional crosslinking agent contains at least 3 unsaturated moieties. Exemplary polyunsaturated compounds include di(meth)acrylate compounds such as ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, 2,2′-bis(4-(acryloxy-propyloxyphenyl)propane, and 2,2′-bis(4-(acryloxydiethoxy-phenyl)propane; tri(meth)acrylate compounds such as, trimethylolpropane tri(meth)acrylate, trimethylolethane tri(meth)acrylate, and tetramethylolmethane tri(meth)acrylate; tetra(meth)acrylate compounds such as ditrimethylolpropane tetra(meth)acrylate, tetramethylolmethane tetra(meth)acrylate, and pentaerythritol tetra(meth)acrylate; hexa(meth)acrylate compounds such as dipentaerythritol hexa(meth)acrylate; allyl compounds such as allyl (meth)acrylate, diallylphthalate, diallyl itaconate, diallyl fumarate, and diallyl maleate; polyallyl ethers of sucrose having from 2 to 8 allyl groups per molecule, polyallyl ethers of pentaerythritol such as pentaerythritol diallyl ether, pentaerythritol triallyl ether, and pentaerythritol tetraallyl ether, and combinations thereof; polyallyl ethers of trimethylolpropane such as trimethylolpropane diallyl ether, trimethylolpropane triallyl ether, and combinations thereof. Other suitable polyunsaturated compounds include divinyl glycol, divinyl benzene, and methylenebisacrylamide.

In another aspect, suitable polyunsaturated monomers can be synthesized via an esterification reaction of a polyol made from ethylene oxide or propylene oxide or combinations thereof with unsaturated anhydride such as maleic anhydride, citraconic anhydride, itaconic anhydride, or an addition reaction with unsaturated isocyanate such as 3-isopropenyl-a-a-dimethylbenzene isocyanate.

Mixtures of two or more of the foregoing polyunsaturated compounds can also be utilized to crosslink the nonionic, amphiphilic polymers. In one aspect, the mixture of conventional unsaturated crosslinking monomer contains an average of 2 unsaturated moieties. In another aspect, the mixture of conventional crosslinking agents contains an average of 2.5 unsaturated moieties. In still another aspect, the mixture of conventional crosslinking agents contains an average of about 3 unsaturated moieties. In a further aspect, the mixture of conventional crosslinking agents contains an average of about 3.5 unsaturated moieties.

In one embodiment, the conventional crosslinking agent component can be used in an amount ranging from about 0.01 to about 1 wt. % in one aspect, from about 0.05 to about 0.75 wt. % in another aspect, and from about 0.1 to about 0.5 wt. % in a further aspect, based on the dry weight of the nonionic, amphiphilic polymer of the disclosed technology.

In another embodiment of the disclosed technology, the conventional crosslinking agent component contains an average of about 3 unsaturated moieties and can be used in an amount ranging from about 0.01 to about 0.3 wt. % in one aspect, from about 0.02 to about 0.25 wt. % in another aspect, from about 0.05 to about 0.2 wt. % in a further aspect, and from about 0.075 to about 0.175 wt. % in a still further aspect, and from about 0.1 to about 0.15 wt. % in another aspect, based upon the total weight of the, nonionic, amphiphilic polymer of the disclosed technology.

In one aspect, the conventional crosslinking agent is selected from trimethylolpropane tri(meth)acrylate, trimethylolethane tri(meth)acrylate, tetramethylolmethane tri(meth)acrylate, pentaerythritol triallylether and polyallyl ethers of sucrose having 3 allyl groups per molecule.

In another aspect, the nonionic amphiphilic polymer can be crosslinked with a combination of a conventional crosslinking agent and an amphiphilic crosslinking agent. The conventional crosslinking agent and amphiphilic crosslinking agent can be used in a total amount ranging from about 0.01 to about 1 wt. % in one aspect, from about 0.05 to about 0.75 wt. % in another aspect, and from about 0.1 to about 0.5 wt. % in a further aspect, based on the dry weight of the nonionic, amphiphilic polymer of the disclosed technology.

In another embodiment, the combination of the conventional crosslinking agent and amphiphilic crosslinking agent can contain an average of about 2 or 3 unsaturated moieties and can be used in an amount ranging from about 0.01 to about 2 wt. % in one aspect, from about 0.02 to about 0.3 wt. % in another aspect, from about 0.05 to about 0.2 wt. % in a further aspect, and from about 0.075 to about 0.175 wt. % in a still further aspect, and from about 0.1 to about 0.15 wt. % in another aspect, based upon the total weight of the, nonionic, amphiphilic polymer of the disclosed technology.

### Amphiphilic Emulsion Polymer Synthesis

The linear and crosslinked, nonionic, amphiphilic emulsion polymers of the disclosed technology can be made using conventional free-radical emulsion polymerization techniques. The polymerization processes are carried out in the absence of oxygen under an inert atmosphere such as nitrogen. The polymerization can be carried out in a suitable solvent system such as water. Minor amounts of a hydrocarbon solvent, organic solvent, as well as mixtures thereof can be employed. The polymerization reactions are initiated by any means which results in the generation of a suitable free-radical. Thermally derived radicals, in which the radical species is generated from thermal, homolytic dissociation of peroxides, hydroperoxides, persulfates, percarbonates, peroxyesters, hydrogen peroxide and azo compounds can be utilized. The initiators can be water soluble or water insoluble depending on the solvent system employed for the polymerization reaction.

The initiator compounds can be utilized in an amount of up to 30 wt. % in one aspect, 0.01 to 10 wt. % in another aspect, and 0.2 to 3 wt. % in a further aspect, based on the total weight of the dry polymer.

Exemplary free radical water soluble initiators include, but are not limited to, inorganic persulfate compounds, such as ammonium persulfate, potassium persulfate, and sodium persulfate; peroxides such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid, and water soluble azo compounds, such as 2,2′-azobis(tert-alkyl) compounds having a water solubilizing substituent on the alkyl group. Exemplary free radical oil soluble compounds include, but are not limited to 2,2′-azobisisobutyronitrile, and the like. The peroxides and peracids can optionally be activated with reducing agents, such as sodium bisulfite, sodium formaldehyde, or ascorbic acid, transition metals, hydrazine, and the like.

In one aspect, azo polymerization catalysts include the Vazo® free-radical polymerization initiators, available from DuPont, such as Vazo® 44 (2,2′-azobis(2-(4,5-dihydroimidazolyl)propane), Vazo® 56 (2,2′-azobis(2-methylpropionamidine) dihydrochloride), Vazo® 67 (2,2′-azobis(2-methylbutyronitrile)), and Vazo® 68 (4,4′-azobis(4-cyanovaleric acid)).

In emulsion polymerization processes, it can be advantageous to stabilize the monomer/polymer droplets or particles by means of surface active auxiliaries. Typically, these are emulsifiers or protective colloids. Emulsifiers used can be anionic, nonionic, cationic or amphoteric. Examples of anionic emulsifiers are alkylbenzenesulfonic acids, sulfonated fatty acids, sulfosuccinates, fatty alcohol sulfates, alkylphenol sulfates and fatty alcohol ether sulfates. Examples of usable nonionic emulsifiers are alkylphenol ethoxylates, primary alcohol ethoxylates, fatty acid ethoxylates, alkanolamide ethoxylates, fatty amine ethoxylates, EO/PO block copolymers and alkylpolyglucosides. Examples of cationic and amphoteric emulsifiers used are quaternized amine alkoxylates, alkylbetaines, alkylamidobetaines and sulfobetaines.

Optionally, the use of known redox initiator systems as polymerization initiators can be employed. Such redox initiator systems include an oxidant (intiator) and a reductant. Suitable oxidants include, for example, hydrogen peroxide, sodium peroxide, potassium peroxide, t-butyl hydroperoxide, t-amyl hydroperoxide, cumene hydroperoxide, sodium perborate, perphosphoric acid and salts thereof, potassium permanganate, and ammonium or alkali metal salts of peroxydisulfuric acid, typically at a level of 0.01% to 3.0% by weight, based on dry polymer weight, are used. Suitable reductants include, for example, alkali metal and ammonium salts of sulfur-containing acids, such as sodium sulfite, bisulfite, thiosulfate, hydrosulfite, sulfide, hydrosulfide or dithionite, formadinesulfinic acid, hydroxymethanesulfonic acid, acetone bisulfite, amines such as ethanolamine, glycolic acid, glyoxylic acid hydrate, ascorbic acid, isoascorbic acid, lactic acid, glyceric acid, malic acid, 2-hydroxy-2-sulfinatoacetic acid, tartaric acid and salts of the preceding acids typically at a level of 0.01% to 3.0% by weight, based on dry polymer weight, is used. In one aspect, combinations of peroxodisulfates with alkali metal or ammonium bisulfites can be used, for example, ammonium peroxodisulfate and ammonium bisulfite. In another aspect, combinations of hydrogen peroxide containing compounds (t-butyl hydroperoxide) as the oxidant with ascorbic or erythorbic acid as the reductant can be utilized. The ratio of peroxide-containing compound to reductant is within the range from 30:1 to 0.05:1.

In emulsion polymerization processes it can be advantageous to stabilize the monomer/polymer droplets or particles by means of surface active auxiliaries. Typically, these are emulsifiers or protective colloids. Emulsifiers used can be anionic, nonionic, cationic or amphoteric. Examples of anionic emulsifiers are alkylbenzenesulfonic acids, sulfonated fatty acids, sulfosuccinates, fatty alcohol sulfates, alkylphenol sulfates and fatty alcohol ether sulfates. Examples of usable nonionic emulsifiers are alkylphenol ethoxylates, primary alcohol ethoxylates, fatty acid ethoxylates, alkanolamide ethoxylates, fatty amine ethoxylates, EO/PO block copolymers and alkylpolyglucosides. Examples of cationic and amphoteric emulsifiers used are quaternized amine alkoxylates, alkylbetaines, alkylamidobetaines and sulfobetaines.

Examples of typical protective colloids are cellulose derivatives, polyethylene glycol, polypropylene glycol, copolymers of ethylene glycol and propylene glycol, polyvinyl acetate, poly(vinyl alcohol), partially hydrolyzed poly(vinyl alcohol), polyvinyl ether, starch and starch derivatives, dextran, polyvinylpyrrolidone, polyvinylpyridine, polyethyleneimine, polyvinylimidazole, polyvinylsuccinimide, polyvinyl-2-methylsuccinimide, polyvinyl-1,3-oxazolid-2-one, polyvinyl-2-methylimidazoline and maleic acid or anhydride copolymers. The emulsifiers or protective colloids are customarily used in concentrations from 0.05 to 20 wt. %, based on the weight of the total monomers.

The polymerization reaction can be carried out at temperatures ranging from 20 to 200° C. in one aspect, from 50 to 150° C. in another aspect, and from 60 to 100° C. in a further aspect.

The polymerization can be carried out the presence of chain transfer agents. Suitable chain transfer agents include, but are not limited to, thio- and disulfide containing compounds, such as C₁-C₁₈ alkyl mercaptans, such as tert-butyl mercaptan, n-octyl mercaptan, n-dodecyl mercaptan, tert-dodecyl mercaptan hexadecyl mercaptan, octadecyl mercaptan; mercaptoalcohols, such as 2-mercaptoethanol, 2-mercaptopropanol; mercaptocarboxylic acids, such as mercaptoacetic acid and 3-mercaptopropionic acid; mercaptocarboxylic acid esters, such as butyl thioglycolate, isooctyl thioglycolate, dodecyl thioglycolate, isooctyl 3-mercaptopropionate, and butyl 3-mercaptopropionate; thioesters; C₁-C₁₈ alkyl disulfides; aryldisulfides; polyfunctional thiols such as trimethylolpropane-tris-(3-mercaptopropionate), pentaerythritol-tetra-(3-mercaptopropionate), pentaerythritol-tetra-(thioglycolate), pentaerythritol-tetra-(thiolactate), dipentaerythritol-hexa-(thioglycolate), and the like; phosphites and hypophosphites; C₁-C₄ aldehydes, such as formaldehyde, acetaldehyde, propionaldehyde; haloalkyl compounds, such as carbon tetrachloride, bromotrichloromethane, and the like; hydroxylammonium salts such as hydroxylammonium sulfate; formic acid; sodium bisulfite; isopropanol; and catalytic chain transfer agents such as, for example, cobalt complexes (e.g., cobalt (II) chelates).

The chain transfer agents are generally used in amounts ranging from 0.1 to 10 wt. %, based on the total weight of the monomers present in the polymerization medium.

### Emulsion Process

In one exemplary aspect of the disclosed technology, the crosslinked, nonionic, amphiphilic emulsion polymer is polymerized via an emulsion process. The emulsion process can be conducted in a single reactor or in multiple reactors as is well-known in the art. The monomers can be added as a batch mixture or each monomer can be metered into the reactor in a staged process. A typical mixture in emulsion polymerization comprises water, monomer(s), an initiator (usually water-soluble) and an emulsifier. The monomers may be emulsion polymerized in a single-stage, two-stage or multi-stage polymerization process according to well-known methods in the emulsion polymerization art. In a two-stage polymerization process, the first stage monomers are added and polymerized first in the aqueous medium, followed by addition and polymerization of the second stage monomers. The aqueous medium optionally can contain an organic solvent. If utilized the organic solvent is less than about 5 wt. % of the aqueous medium. Suitable examples of water-miscible organic solvents include, without limitation, esters, alkylene glycol ethers, alkylene glycol ether esters, lower molecular weight aliphatic alcohols, and the like.

To facilitate emulsification of the monomer mixture, the emulsion polymerization is carried out in the presence of at least one surfactant. In one embodiment, the emulsion polymerization is carried out in the presence of surfactant (active weight basis) ranging in the amount of about 0.2% to about 5% by weight in one aspect, from about 0.5% to about 3% in another aspect, and from about 1% to about 2% by weight in a further aspect, based on a total monomer weight basis. The emulsion polymerization reaction mixture also includes one or more free radical initiators which are present in an amount ranging from about 0.01% to about 3% by weight based on total monomer weight. The polymerization can be performed in an aqueous or aqueous alcohol medium. Surfactants for facilitating the emulsion polymerization include anionic, nonionic, amphoteric, and cationic surfactants, as well as mixtures thereof. Most commonly, anionic and nonionic surfactants can be utilized as well as mixtures thereof.

Suitable anionic surfactants for facilitating emulsion polymerizations are well known in the art and include, but are not limited to (C₆-C₁₈) alkyl sulfates, (C₆-C₁₈) alkyl ether sulfates (e.g., sodium lauryl sulfate and sodium laureth sulfate), amino and alkali metal salts of dodecylbenzenesulfonic acid, such as sodium dodecyl benzene sulfonate and dimethylethanolamine dodecylbenzenesulfonate, sodium (C₆-C₁₆) alkyl phenoxy benzene sulfonate, disodium (C₆-C₁₆) alkyl phenoxy benzene sulfonate, disodium (C₆-C₁₆) di-alkyl phenoxy benzene sulfonate, disodium laureth-3 sulfosuccinate, sodium dioctyl sulfosuccinate, sodium di-sec-butyl naphthalene sulfonate, disodium dodecyl diphenyl ether sulfonate, disodium n-octadecyl sulfosuccinate, phosphate esters of branched alcohol ethoxylates, and the like.

Nonionic surfactants suitable for facilitating emulsion polymerizations are well known in the polymer art, and include, without limitation, linear or branched C₈-C₃₀ fatty alcohol ethoxylates, such as capryl alcohol ethoxylate, lauryl alcohol ethoxylate, myristyl alcohol ethoxylate, cetyl alcohol ethoxylate, stearyl alcohol ethoxylate, cetearyl alcohol ethoxylate, sterol ethoxylate, oleyl alcohol ethoxylate, and, behenyl alcohol ethoxylate; alkylphenol alkoxylates, such as octylphenol ethoxylates; and polyoxyethylene polyoxypropylene block copolymers, and the like. Additional fatty alcohol ethoxylates suitable as non-ionic surfactants are described below. Other useful nonionic surfactants include 08-022 fatty acid esters of polyoxyethylene glycol, ethoxylated mono- and diglycerides, sorbitan esters and ethoxylated sorbitan esters, 08-022 fatty acid glycol esters, block copolymers of ethylene oxide and propylene oxide, and combinations thereof. The number of ethylene oxide units in each of the foregoing ethoxylates can range from 2 and above in one aspect, and from 2 to about 150 in another aspect.

Optionally, other emulsion polymerization additives and processing aids which are well known in the emulsion polymerization art, such as auxiliary emulsifiers, protective colloids, solvents, buffering agents, chelating agents, inorganic electrolytes, polymeric stabilizers, biocides, and pH adjusting agents can be included in the polymerization system.

In one embodiment of the disclosed technology, the protective colloid or auxiliary emulsifier is selected from poly(vinyl alcohol) that has a degree of hydrolysis ranging from about 80 to 95% in one aspect, and from about 85 to 90% in another aspect.

In a typical two stage emulsion polymerization, a mixture of the monomers is added to a first reactor under inert atmosphere to a solution of emulsifying surfactant (e.g., anionic surfactant) in water. Optional processing aids can be added as desired (e.g., protective colloids, auxiliary emulsifier(s)). The contents of the reactor are agitated to prepare a monomer emulsion. To a second reactor equipped with an agitator, an inert gas inlet, and feed pumps are added under inert atmosphere a desired amount of water and additional anionic surfactant and optional processing aids. The contents of the second reactor are heated with mixing agitation. After the contents of the second reactor reaches a temperature in the range of about 55 to 98° C., a free radical initiator is injected into the so formed aqueous surfactant solution in the second reactor, and the monomer emulsion from the first reactor is gradually metered into the second reactor over a period typically ranging from about one half to about four hours. The reaction temperature is controlled in the range of about 45 to about 95° C. After completion of the monomer addition, an additional quantity of free radical initiator can optionally be added to the second reactor, and the resulting reaction mixture is typically held at a temperature of about 45 to 95° C. for a time period sufficient to complete the polymerization reaction to obtain the polymer emulsion.

In one aspect, the crosslinked, nonionic, amphiphilic polymers of the disclosed technology are selected from an emulsion polymer polymerized from a monomer mixture comprising from about 20 to about 60 wt. % of at least one C₁-C₄ hydroxyalkyl (meth)acrylate (e.g., hydroxyethyl methacrylate); from about 10 to about 70 wt. % of at least one C₁-C₀₁2 alkyl (meth)acrylate in one aspect or from about 10 to about 70 wt. % of at least one C₁-C₀₅ alkyl (meth)acrylate in another aspect; from about 0, 1, 5 or 15 to about 40 wt. % of at least one vinyl ester of a C₁-C₁₀ carboxylic acid, from about 0, 1 or 15 to about 30 wt. % of a vinyl lactam (e.g., vinyl pyrrolidone); from about 0, 0.1, 1, 5, or 7 to about 15 wt. % of at least one associative and/or a semi-hydrophobic monomer (wherein all monomer weight percentages are based on the weight of the total monomers); and from about 0.01 to about 5 wt. % in one aspect, from about 0.1 to about 3 in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In another aspect, the crosslinked, nonionic, amphiphilic polymers of the disclosed technology are selected from an emulsion polymer polymerized from a monomer mixture comprising from about 20 to about 50 wt. % at least one C₁-C₄ hydroxyalkyl (meth)acrylate (e.g., hydroxyethyl methacrylate); from about 10 to about 30 wt. % ethyl acrylate; from about 10 to about 35 wt. % butyl acrylate; from about 0 or 15 to about 25 wt. % of a vinyl ester of a C₁-C₅ carboxylic acid selected from vinyl formate, vinyl acetate, vinyl propionate, vinyl butyrate, vinyl isobutyrate, and vinyl valerate; from about 0, 1 or 15 to about 30 wt. % of vinyl pyrrolidone; and from about 0, 0.1, 1, 5 or 7 to about 15 wt. % of at least one associative monomer and/or semi-hydrophobic monomer (wherein all monomer weight percentages are based on the weight of the total monomers); and from about 0.01 to about 5 wt. % in one aspect, from about 0.1 to about 3 in another aspect, and from about 0.5 to about 1 in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In another embodiment, the crosslinked, nonionic, amphiphilic polymers of the disclosed technology are selected from an emulsion polymer polymerized from a monomer mixture comprising from about 20 to about 50 wt. % of hydroxyethyl methacrylate; from about 10 to about 30 wt. % ethyl acrylate; from about 10 to about 30 wt. % butyl acrylate; from about 0, 1, or 15 to about 25 wt. % of vinyl pyrrolidone; from about 0 or 15 to about 25 wt. % of vinyl acetate; from about 0, 0.1, 1, 5 or 7 to about 10 wt. % of at least one associative and/or semi-hydrophobic monomer (wherein all monomer weight percentages are based on the weight of the total monomers); and from about 0.01 to about 5 wt. % in one aspect, from about 0.1 to about 3 wt. % in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In another embodiment, the crosslinked, nonionic, amphiphilic polymers of the disclosed technology are selected from an emulsion polymer polymerized from a monomer mixture comprising from about 20 to 50 wt. % of hydroxyethyl methacrylate; from about 10 to about 40 wt. % ethyl acrylate; from about 10 to about 20 wt. % butyl acrylate; from about 0.1 to about 10 wt. % of at least one associative and/or semi-hydrophobic monomer (wherein all monomer weight percentages are based on the weight of the total monomers); and from about 0.01 to about 5 wt. % in one aspect, from about 0.1 to about 3 wt. % in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In one aspect, the crosslinked, nonionic, amphiphilic polymers of the disclosed technology are selected from an emulsion polymer polymerized from a monomer mixture comprising from about 20 to 50 wt. % of hydroxyethyl methacrylate; from about 10 to about 30 wt. % ethyl acrylate; from about 10 to about 30 wt. % butyl acrylate; from about 1 to about 10 wt. % of at least one associative and/or semi-hydrophobic monomer (wherein all monomer weight percentages are based on the weight of the total monomers); and from about 0.01 to about 5 wt. % in one aspect, from about 0.1 to about 3 wt. % in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In one aspect, the crosslinked, nonionic, amphiphilic polymers of the disclosed technology are selected from an emulsion polymer polymerized from a monomer mixture comprising from about 20 to 35 wt. % of hydroxyethyl methacrylate, from about 10 to about 30 wt. % ethyl acrylate, from about 10 to about 30 wt. % butyl acrylate, from about 15 to about 25 wt. % of vinyl pyrrolidone, from about 15 to about 25 wt. % of vinyl acetate (wherein all monomer weight percentages are based on the weight of the total monomers), and from about 0.01 to about 5 wt. % in one aspect, from about 0.1 to about 3 wt. % in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In one aspect, the crosslinked, nonionic, amphiphilic polymers of the disclosed technology are selected from an emulsion polymer polymerized from a monomer mixture comprising from about 20 to 40 wt. % of hydroxyethyl methacrylate, from about 10 to about 30 wt. % ethyl acrylate, from about 10 to about 30 wt. % butyl acrylate, from about 15 to about 25 wt. % of vinyl pyrrolidone, and from about 1 to about 5 wt. % of at least one associative and/or semi-hydrophobic monomer (wherein all monomer weight percentages are based on the weight of the total monomers), and from about 0.01 to about 5 wt. % in one aspect, from about 0.1 to about 3 wt. % in another aspect, and from about 0.5 to about 1 wt. % in a further aspect of at least one crosslinker (based on the dry weight of the polymer), wherein the at least one crosslinker is selected from an amphiphilic crosslinking agent or a combination of an amphiphilic crosslinking agent and a conventional crosslinking agent as defined herein.

In another aspect, the at least one nonionic, amphiphilic emulsion polymer utilized in formulating the hair care compositions of the disclosed technology is crosslinked. The crosslinked nonionic, amphiphilic emulsion polymers of the technology are random copolymers and have weight average molecular weights ranging from above about 500,000 to at least about a billion Daltons or more in one aspect, and from about 600,000 to about 4.5 billion Daltons in another aspect, and from about 1,000,000 to about 3,000,000 Daltons in a further aspect, and from about 1,500,000 to about 2,000,000 Daltons in a still further aspect (see TDS-222, Oct. 15, 2007, Lubrizol Advanced Materials, Inc., which is herein incorporated by reference).

### B. Antidandruff Agents

The antidandruff agents of the present technology are any particulate compound capable of relieving the symptoms of dandruff and that are substantive to the hair, scalp and skin to afford residual antidandruff properties between shampoos. Among the many particulate compounds exhibiting antidandruff properties that are useful herein are salicylic acid, elemental sulfur, selenium sulfides, azole compounds, 2-pyridone derivatives based on 1-hydroxy-2-pyridone, and polyvalent metal salts of pyrithione.

Sulfur is a particulate antidandruff agent that is effective in the compositions of the disclosed technology. Sulfur can be utilized in an amount ranging from about 1 wt. % to about 5 wt. % in one aspect, and from about 2 wt. % to about 4 wt. % in another aspect, based on the weight of the total composition.

Selenium sulfide is a particulate anti-dandruff agent suitable for use in the antidandruff compositions of the present technology and is selected from compounds of the formula Se₈₋ₓSₓ where x is a number ranging from 1 to 7. Effective concentrations of selenium sulfide can range from about 0.1% to about 4 wt. % in one aspect, from about 0.3% to about 2.5 wt. % in another aspect, and from about 0.5% to about 1.5 wt. % in still another aspect, based on the weight of the composition.

The azole antidandruff agents include imidazoles such as benzimidazole, benzothiazole, bifonazole, butoconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole antidandruff agent can be included in an amount from about 0.01% to about 5 wt. % in one aspect, from about 0.1% to about 3 wt. % in another aspect, and from about 0.3% to about 2 wt. % in still another aspect, based on the weight of the composition.

Exemplary antidandruff agents that are based on 1-hydroxy-2-pyridone are 1-hydroxy-4-methyl-2-pyridone, 1-hydroxy-6-methylpyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methyl-cyclohexyl)₂-pyridone, 1-hydroxy-4-methyl-6-(2-bicyclo[2,2,1]heptyl)-2-pyridone, 1-hydroxy-4-methyl-6 (4-methylphenyl)-2-pyridone, 1-hydroxy-4-methyl-6 [1-[4-nitrophenoxy]-butyl]-2-pyridone, 1-hydroxy-4-methyl-6-(4-cyanophenoxymethyl-2-pyridone), 1-hydroxy-4-methyl-6-(phenylsulfonylmethyl)-2-pyridone, 1-hydroxy-4-methyl-6-(4-bromobenzyl)-2-pyridone and salts thereof. In one embodiment, the monoethanolamine salt of 1-hydroxy-4-methyl-6-(2,4,4-trimethylpenthyl)-2-pyridone, monoethanolamine salt available from Clariant under the trade name Octopirox® is a suitable antidandruff agent.

The polyvalent metal salts of pyrithione include those formed from the polyvalent metals magnesium, barium, bismuth, strontium, copper, zinc, cadmium, zirconium and mixtures thereof. The polyvalent metal salts of pyrithione can be represented by Formula X as follows:

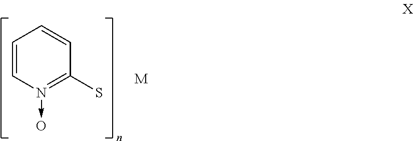

in which M is a polyvalent metal ion selected from magnesium, barium, bismuth, strontium, copper, zinc, cadmium and zirconium, and n corresponds to the valency of M. Any physical form of polyvalent metal pyrithione salts can be used, including platelet and needle configurations.

In one embodiment the polyvalent metal salt of pyrithione is selected from the zinc salt of 1-hydroxy-2-pyridinethione, i.e., the zinc complex of 2-pyridinethiol-1-oxide (known as “zinc pyrithione” or “ZPT”) represented by Formula XA as follows:

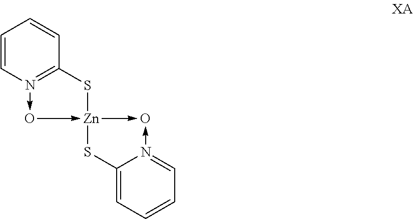

In one aspect the ZPT antidandruff agent has an average particle size of up to about 20 μm in one aspect, up to about 5 μm in another aspect, up to about 2.5 μm in still another aspect, and up to about 1 μm in a further aspect. In an additional embodiment the average particle size can range from about 0.1 μm to about 1 μm in one aspect, and from about 0.25 μm to about 0.75 μm in another aspect. The average particle size can be measured by light scattering techniques well-known in the art for determining average particle size for particulate materials. One such method involves measuring particle size by means of a laser light scattering technique using a Horiba model LA 910 laser scattering particle size distribution analyzer (Horiba Instruments, Inc., Irvine, Calif.).

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. Zinc pyrithione can be made by reacting 1-hydroxy-2-pyridinethione (i.e., pyrithione acid) or a soluble salt thereof with a zinc salt (e.g. zinc sulfate) to form a zinc pyrithione precipitate, as illustrated in U.S. Pat. No. 2,809,971. Zinc pyrithione is commercially available from Arch Chemicals, Inc. (Lonza Group Ltd.), under the trade name Zinc Ormadine™

In one embodiment the amount of polyvalent metal salt of pyrithione (e.g., ZPT) suitable for use in the compositions of the present technology range from about 0.01 wt. % to about 5 wt. % in one aspect, and from about 0.1 wt. % to about 2 wt. % in another aspect, based on the weight of the composition.

In one embodiment of the present technology, the polyvalent metal salt of pyrithione can be used in combination with a secondary particulate zinc salt as disclosed in U.S. Pat. Application Pub. No. 2004/0213751 and U.S. Pat. No. 8,491,877, the pertinent disclosures of which are incorporated herein by reference. It is disclosed that zinc containing layered materials (ZLM) are useful secondary salts that augment the antimicrobial efficacy of polyvalent metal salts of pyrithione, particularly ZPT.

Exemplary ZLM's include, but are not limited to, hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions.

In one embodiment, basic zinc carbonate is used in combination with ZPT. Basic zinc carbonate, which also is referred to commercially as “zinc carbonate” or “zinc carbonate basic” or “zinc hydroxy carbonate”, is a synthetic version consisting of materials similar to naturally occurring hydrozincite. The idealized stoichiometry is represented as Zn₅(OH)₆(CO₃)₂, but the actual stoichiometric ratios can vary slightly and other impurities may be incorporated in the crystal lattice. Commercially available sources of basic Zinc Carbonate include Zinc Carbonate Basic (Cater Chemicals: Bensenville, Ill., USA), Zinc Carbonate Basic (Sigma-Aldrich: St. Louis, Mo., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

In aspect of the present technology, the ZLM (e.g., basic zinc carbonate) can have a particle size distribution wherein 90% of the particles are less than about 50 μm. In another aspect, the ZLM can have a particle size distribution wherein 90% of the particles are less than about 30 μm. In yet a further aspect, the ZLM can have a particle size distribution wherein 90% of the particles are less than about 20 μm.

In another aspect of the present technology, the ZLM (e.g., basic zinc carbonate) can have a surface area of greater than about 10 m²/gm. In a further aspect, the ZLM can have a surface area of greater than about 20 m²/gm. In yet a further aspect of the ZLM can have a surface area of greater than about 30 m²/gm.

In embodiments utilizing a ZLM and a polyvalent metal salt of pyrithione (e.g. ZPT), the ratio of ZLM to polyvalent metal salt of pyrithione is from about 5:100 to about 10:1 in one aspect, from about 2:10 to about 5:1 in another aspect, and from about 1:2 to about 3:1 in still another aspect (all ratios based on a wt./wt. basis).

In one embodiment of the present technology, the polyvalent metal salt of pyrithione can be used in combination with a metal ion source such as copper and zinc salts, as disclosed in International Pat. Application Pub. No. WO 01/00151, which is incorporated by reference for the pertinent disclosure therein. It is disclosed that antidandruff efficacy can be dramatically increased in topical compositions by the use of polyvalent metal salts of pyrithione, such as ZPT, in combination with a metal ion source such as copper and zinc salts. The metal ion source may be selected from zinc, copper, silver, nickel, cadmium, mercury, and bismuth. In one aspect, the metal ion is selected from zinc salts, copper salts, silver salts, and mixtures thereof.

In one aspect, the metal ion is selected from zinc salts, copper salts, and mixtures thereof. Exemplary metal ion salts of zinc and copper include, but are not limited to, zinc acetate, zinc oxide, zinc carbonate, zinc hydroxide, zinc chloride, zinc sulfate, zinc citrate, zinc fluoride, zinc iodide, zinc lactate, zinc oleate, zinc oxalate, zinc phosphate, zinc propionate, zinc salicylate, zinc selenate, zinc silicate, zinc stearate, zinc sulfide, zinc tannate, zinc tartrate, zinc valerate, zinc gluconate, zinc undecylate, and the like. Combinations of zinc salts may also be used in the composition of the disclosed technology Exemplary metal ion salts of copper include, but are not limited to, copper disodium citrate, copper triethanolamine, copper carbonate, cuprous ammonium carbonate, cupric hydroxide, copper chloride, cupric chloride, copper ethylenediamine complex, copper oxychlon'de, copper oxychloride sulfate, cuprous oxide, copper thiocyanate, and the like. Combinations of these copper salts may also be used in the composition of the disclosed technology.

The metal ion source is present in the composition in a ratio (wt./wt.) to polyvalent metal salt of pyrithione of from about 5:100 to about 5:1 in one aspect, from about 2:10 to about 3:1 in another aspect, and from about 1:2 to about 2:1 in still another aspect.

### C. Detersive Surfactants

The surfactants utilized to formulate the hair care compositions of the disclosed technology are chosen from at least one detersive surfactant selected from at least one anionic surfactant, and an optional surfactant selected from amphoteric or zwitterionic surfactants, nonionic surfactants, and mixtures thereof.

Non-limiting examples of anionic surfactants are disclosed in McCutcheon's Detergents and Emulsifiers, North American Edition, 1998, published by Allured Publishing Corporation; and McCutcheon's, Functional Materials, North American Edition (1992); both of which are incorporated by reference herein in their entirety. The anionic surfactant can be any of the anionic surfactants known or previously used in the art of aqueous surfactant compositions, including synthetic surfactants (syndets) and fatty acid soaps.

Suitable anionic syndet surfactants include but are not limited to alkyl sulfates, alkyl ether sulfates alkyl sulfonates, alkylaryl sulfonates, alkenyl and hydroxyalkyl alpha-olefin-sulfonates, and mixtures thereof, alkylamide sulfonates, alkarylpolyether sulphates, alkylamidoether sulphates, alkyl and alkenyl monoglyceryl ether sulfates, alkyl and alkenyl monoglyceride sulfates, alkyl and alkenyl monoglyceride sulfonates, alkyl and alkenyl succinates, alkyl and alkenyl sulfosuccinates, alkyl and alkenyl sulfosuccinamates, alkyl and alkenyl ether sulfosuccinates, alkyl and alkenyl amidosulfosuccinates; alkyl and alkenyl sulphoacetates, alkyl and alkenyl phosphates, alkyl and alkenyl ether phosphates, alkyl and alkenyl carboxylates, alkyl and alkenyl ether carboxylates, alkyl and alkenyl amidoethercarboxylates, N-alkylamino acids, N-acyl amino acids, alkyl peptides, N-acyl taurates, acyl isethionates, carboxylate salts wherein the acyl group is derived from fatty acids; and the alkali metal, alkaline earth metal, ammonium, amine, and triethanolamine salts thereof.

In one aspect, the cation moiety of the forgoing salts is selected from sodium, potassium, magnesium, ammonium, mono-, di- and triethanolamine salts, and mono-, di-, and tri-isopropylamine salts. The alkyl and acyl groups of the foregoing surfactants contain from about 6 to about 24 carbon atoms in one aspect, from 8 to 22 carbon atoms in another aspect, and from about 12 to 18 carbon atoms in a further aspect, and can be saturated or unsaturated. The aryl groups in the surfactants are selected from phenyl or benzyl. The ether containing surfactants set forth above can contain from 1 to 10 ethylene oxide and/or propylene oxide units per surfactant molecule in one aspect, and from 1 to 3 ethylene oxide units per surfactant molecule in another aspect.

Examples of suitable anionic surfactants include but are not limited to the sodium, potassium, lithium, magnesium, ammonium, and triethanolamine lauryl sulfate, coco sulfate, tridecyl sulfate, myrstyl sulfate, cetyl sulfate, cetearyl sulfate, stearyl sulfate, oleyl sulfate, and tallow sulfate; the sodium, potassium, lithium, magnesium, and ammonium salts of laureth sulfate, trideceth sulfate, myreth sulfate, C₁₂-C₁₃ pareth sulfate, C₁₂-C₁₄ pareth sulfate, and C₁₂-C₁₅ pareth sulfate, ethoxylated with 1, 2, 3, 4 or 5 moles of ethylene oxide; disodium lauryl sulfosuccinate, disodium laureth sulfosuccinate, sodium cocoyl isethionate, sodium C₁₂-C₁₄ olefin sulfonate, sodium laureth-6 carboxylate, sodium methyl cocoyl taurate, sodium cocoyl glycinate, sodium myristyl sarcocinate, sodium dodecylbenzene sulfonate, sodium cocoyl sarcosinate, sodium cocoyl glutamate, potassium myristoyl glutamate, triethanolamine monolauryl phosphate, and fatty acid soaps, including the sodium, potassium, ammonium, and triethanolamine salts of a saturated and unsaturated fatty acids containing from about 8 to about 22 carbon atoms.

The anionic fatty acid soaps are salts of fatty acids containing from about 8 to about 22 carbon atoms, and mixtures thereof. In another aspect, the fatty acid soap contains from about 10 to about 18 carbon atoms, and mixtures thereof. In a further aspect, the fatty acid soap contains from about 12 to about 16 carbon atoms, and mixtures thereof. The fatty acids utilized in the soaps can be saturated and unsaturated and can be derived from synthetic sources, as well as from the hydrolysis of fats and natural oils.

Exemplary saturated fatty acids include but are not limited to octanoic, decanoic, lauric, myristic, pentadecanoic, palmitic, margaric, steric, isostearic, nonadecanoic, arachidic, behenic, and the like, and mixtures thereof. Exemplary unsaturated fatty acids include but are not limited to myristoleic, palmitoleic, oleic, linoleic, linolenic, and the like, and mixtures thereof. The fatty acids can be derived from animal fat such as tallow, lard, poultry fat or from vegetable sources such as coconut oil, red oil, palm kernel oil, palm oil, cottonseed oil, linseed oil, sunflower seed oil, olive oil, soybean oil, peanut oil, corn oil, safflower oil, sesame oil, rapeseed oil, canola oil, and mixtures thereof.

The soap can be prepared by a variety of well-known means such as by the direct base neutralization of a fatty acid or mixtures thereof or by the saponification of suitable fats and vegetable oils or mixtures thereof with a suitable base. Exemplary bases include potassium hydroxide, potassium carbonate, sodium hydroxide and alkanol amines such as triethanolamine. Generally, the fat or oil is heated until liquefied and a solution of the desired base is added thereto. Soaps included in a composition utilized in the method of the disclosed technology can be made, for example, by a classic kettle process or modern continuous manufacturing process wherein natural fats and oils such as tallow or coconut oil or their equivalents are saponified with an alkali metal hydroxide using procedures well known to those skilled in the art. Alternatively, soaps can be made by the direct neutralization of free fatty acids such as lauric acid (C₁₂), myristic acid (C₁₄), palmitic acid (C₁₆), steric acid (C₁₈), isostearic (C₁₈), and mixtures thereof, with an alkali metal hydroxide or carbonate.

The anionic of the anionic surfactant component in the composition should be sufficient to provide the desired cleansing and lather performance, and generally ranges from about 2 wt. % to about 50 wt. % in one aspect, from about 8 wt. % to about 30 wt. % in another aspect, from about 10 wt. % to about 25 wt. % in still another aspect, and from about 12 wt. % to about 22 wt. % in a further aspect, all weight percentages are based on the weight of the total composition.

The term “amphoteric surfactant” as used herein, is also intended to encompass zwitterionic surfactants, which are well known to formulators skilled in the art as a subset of amphoteric surfactants. Non-limiting examples of amphoteric surfactants are disclosed McCutcheon's Detergents and Emulsifiers, North American Edition, supra, and McCutcheon's, Functional Materials, North American Edition, supra; both of which are incorporated by reference herein in their entirety. Suitable examples include but are not limited to amino acids (e.g., N-alkyl amino acids and N-acyl amino acids), betaines, sultaines, and alkyl amphocarboxylates. Other non-limiting examples of suitable zwitterionic or amphoteric surfactants are described in U.S. Pat. Nos. 5,104,646, and 5,106,609.

Amino acid based surfactants suitable in the practice of the present technology include surfactants represented by the formula:

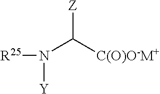

wherein R²⁵ represents a saturated or unsaturated hydrocarbon group having 10 to 22 carbon atoms or an acyl group containing a saturated or unsaturated hydrocarbon group having 9 to 22 carbon atoms, Y is hydrogen or methyl, Z is selected from hydrogen, —CH₃, —CH(CH₃)₂, —CH₂CH(CH₃)₂, —CH(CH₃)CH₂CH₃, —CH₂C₆H₅, —CH₂C₆H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)O⁻M⁺. M is a salt forming cation. In one aspect, R²⁵ represents a radical selected from a linear or branched C₁₀ to C₂₂ alkyl group, a linear or branched C₁₀ to C₂₂ alkenyl group, an acyl group represented by R²⁶C(O)—, wherein R²⁶ is selected from a linear or branched C₉ to C₂₂ alkyl group, a linear or branched C₉ to C₂₂ alkenyl group. In one aspect, M⁺ is a cation selected from sodium, potassium, ammonium, and the ammonium salt of mono-, di, and triethanolamine (TEA).

The amino acid surfactants can be derived from the alkylation and acylation of a-amino acids such as, for example, alanine, arginine, aspartic acid, glutamic acid, glycine, isoleucine, leucine, lysine, phenylalanine, serine, tyrosine, and valine. Representative N-acyl amino acid surfactants are, but not limited to the mono- and di-carboxylate salts (e.g., sodium, potassium, ammonium and TEA) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and TEA) of N-acylated alanine, for example, sodium cocoyl alaninate, and TEA lauroyl alaninate; the carboxylate salts (e.g., sodium, potassium, ammonium and TEA) of N-acylated glycine, for example, sodium cocoyl glycinate, and potassium cocoyl glycinate; the carboxylate salts (e.g., sodium, potassium, ammonium and TEA) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate; and mixtures of the foregoing surfactants.

The betaines and sultaines useful in the present technology are selected from alkyl betaines, alkylamino betaines, and alkylamido betaines, as well as the corresponding sulfobetaines (sultaines) represented by the formulas:

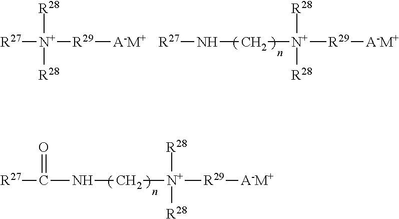

wherein R²⁷ is a C₇-C₂₂ alkyl or alkenyl group, each R²⁸ independently is a C₁-C₄ alkyl group, R²⁹ is a C₁-C₅ alkylene group or a hydroxy substituted C₁-C₅ alkylene group, n is an integer from 2 to 6, A is a carboxylate or sulfonate group, and M is a salt forming cation. In one aspect, R²⁷ is a C₁₁-C₁₈ alkyl group or a C₁₁-C₁₈ alkenyl group. In one aspect, R²⁸ is methyl. In one aspect, R²⁹ is methylene, ethylene or hydroxy propylene. In one aspect, n is 3. In a further aspect, M is selected from sodium, potassium, magnesium, ammonium, and mono-, di- and triethanolamine cations.

Examples of suitable betaines include, but are not limited to, lauryl betaine, coco betaine, oleyl betaine, coco hexadecyl dimethylbetaine, coco dimethyl carboxymethyl betaine, lauryl dimethyl carboxymethyl betaine, cetyl dimethyl carboxymethyl betaine, lauryl amidopropyl betaine, cocoamidopropyl betaine (CAPB), coco dimethyl sulfopropyl betaine, stearyl dimethyl sulfopropyl betaine, lauryl dimethyl sulfoethyl betaine, and cocamidopropyl hydroxysultaine.

The alkylamphocarboxylates such as the alkylamphoacetates and alkylamphopropionates (mono- and disubstituted carboxylates) can be represented by the formula:

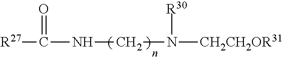

wherein R²⁷ is a C₇-C₂₂ alkyl or alkenyl group, R³⁰ is —CH₂C(O)O⁻ M⁺, —CH₂CH₂C(O)O⁻ M⁺, or —CH₂CH(OH)CH₂SO₃⁻ M⁺, R³¹ is hydrogen or —CH₂C(O)O⁻ M⁺, and M is a cation selected from sodium, potassium, magnesium, ammonium, and the ammonium salt of mono-, di- and triethanolamine.

Exemplary alkylamphocarboxylates include, but are not limited to, sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

The amount of such amphoteric or zwitterionic detersive surfactants ranges from about 0.5 wt. % to about 20 wt. % in one aspect, and from about 1 wt. % to about 10 wt. % in another aspect, based on the weight of the total composition.

Non-limiting examples of nonionic surfactants are disclosed in McCutcheon's Detergents and Emulsifiers, North American Edition, 1998, supra; and McCutcheon's, Functional Materials, North American, supra; both of which are incorporated by reference herein in their entirety. Additional Examples of nonionic surfactants are described in U.S. Pat. No. 4,285,841, to Barrat et al., and U.S. Pat. No. 4,284,532, to Leikhim et al., both of which are incorporated by reference herein in their entirety. Nonionic surfactants typically have a hydrophobic portion, such as a long chain alkyl group or an alkylated aryl group, and a hydrophilic portion containing various degrees of ethoxylation and/or propoxylation (e.g., 1 to about 50) ethoxy and/or propoxy moieties. Examples of some classes of nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty acids, condensation products of ethylene oxide with long chain amines or amides, condensation products of ethylene oxide with alcohols, and mixtures thereof.

Suitable nonionic surfactants include, for example, alkyl polysaccharides, alcohol ethoxylates, block copolymers, castor oil ethoxylates, ceto/oleyl alcohol ethoxylates, cetearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, tallow oil fatty acid ethoxylates, tallow amine ethoxylates, tridecanol ethoxylates, acetylenic diols, polyoxyethylene sorbitols, and mixtures thereof. Various specific examples of suitable nonionic surfactants include, but are not limited to, Cocamide MEA, Cocamide MIPA, methyl gluceth-10, PEG-20 methyl glucose distearate, PEG-20 methyl glucose sesquistearate, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, poloxamers such as poloxamer 188, polysorbate 21, polysorbate 40, polysorbate 60, polysorbate 61, polysorbate 65, polysorbate 80, polysorbate 81, polysorbate 85, sorbitan caprylate, sorbitan cocoate, sorbitan diisostearate, sorbitan dioleate, sorbitan distearate, sorbitan fatty acid ester, sorbitan isostearate, sorbitan laurate, sorbitan oleate, sorbitan palmitate, sorbitan sesquiisostearate, sorbitan sesquioleate, sorbitan sesquistearate, sorbitan stearate, sorbitan triisostearate, sorbitan trioleate, sorbitan tristearate, sorbitan undecylenate, or mixtures thereof.

Alkyl glycoside nonionic surfactants can also be employed and are generally prepared by reacting a monosaccharide, or a compound hydrolyzable to a monosaccharide, with an alcohol such as a fatty alcohol in an acid medium. For example, U.S. Pat. Nos. 5,527,892 and 5,770,543 describe alkyl glycosides and/or methods for their preparation. Suitable examples are commercially available under the names of Glucopon™ 220, 225, 425, 600 and 625, PLANTACARE®, and PLANTAPON®, all of which are available from Cognis Corporation.

In another aspect, nonionic surfactants include, but are not limited to, alkoxylated methyl glucosides such as, for example, methyl gluceth-10, methyl gluceth-20, PPG-10 methyl glucose ether, and PPG-20 methyl glucose ether, available from Lubrizol Advanced Materials, Inc., under the trade names, Glucam® E10, Glucam® E20, Glucam® P10, and Glucam® P20, respectively; and hydrophobically modified alkoxylated methyl glucosides, such as PEG 120 methyl glucose dioleate, PEG-120 methyl glucose trioleate, and PEG-20 methyl glucose sesquistearate, available from Lubrizol Advanced Materials, Inc., under the trade names, Glucamate® DOE-120, Glucamate™ LT, and Glucamate™ SSE-20, respectively, are also suitable. Other exemplary hydrophobically modified alkoxylated methyl glucosides are disclosed in U.S. Pat. Nos. 6,573,375 and 6,727,357, the relevant disclosure of which are hereby incorporated by reference.

Other useful nonionic surfactants include water soluble silicones such as PEG-10 Dimethicone, PEG-12 Dimethicone, PEG-14 Dimethicone, PEG-17 Dimethicone, PPG-12 Dimethicone, PPG-17 Dimethicone and derivatized/functionalized forms thereof such as Bis-PEG/PPG-20/20 Dimethicone Bis-PEG/PPG-16/16 PEG/PPG-16/16 Dimethicone, PEG/PPG-14/4 Dimethicone, PEG/PPG-20/20 Dimethicone, PEG/PPG-20/23 Dimethicone, and Perfluorononylethyl Carboxydecyl PEG-10 Dimethicone.

In one embodiment of the disclosed technology, at least one anionic surfactant is utilized in combination with an amphoteric or zwitterionic surfactant. In one aspect, the weight ratio (based on active material) of anionic surfactant (non-ethoxylated and/or ethoxylated) to amphoteric surfactant can range from about 10:1 to about 2:1 in one aspect, and can be about 9:1, about 8:1, about 7:1, about 6:1, about 5:1, about 4.5:1, about 4:1, or about 3:1 in another aspect. When employing an ethoxylated anionic surfactant in combination with a non-ethoxylated anionic surfactant and an amphoteric or zwitterionic surfactant, the weight ratio (based on active material) of ethoxylated anionic surfactant to non-ethoxylated anionic surfactant to amphoteric surfactant can range from about 3.5:3.5:1 in one aspect to about 1:1:1 in another aspect.

In one aspect, the anionic surfactant is selected from alkyl sulfates, including sodium lauryl sulfate, ammonium lauryl sulfate, sodium coco-sulfate, and mixtures thereof.

In one aspect, the anionic surfactant is selected from ethoxylated alkyl sulfates including sodium laureth sulfate, ammonium laureth sulfate, sodium trideceth sulfate, and mixtures thereof.

In one aspect, the optional amphoteric surfactant is selected from alkyl betaines, amidoalkyl betaines and amidoalkyl sultaines including lauryl betaine, cocamidopropyl betaine, cocamidopropyl hydroxysultaine, and mixtures thereof.

### D. Aqueous Carrier

The compositions of the present technology are typically in the form of pourable liquids (under ambient conditions). The compositions will therefore typically comprise an aqueous carrier, which is present at a level of from about 20 wt. % to about 95 wt. % in one aspect, and from about 60 wt. % to about 85 wt. % in another aspect, based on the weight of the total composition. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

### E. Optional Components

The compositions of the present technology may further comprise one or more optional components known for use in hair care or personal care products, provided that the optional components are physically and chemically compatible with the essential components described herein, or do not otherwise unduly impair product stability, aesthetics or performance. Unless otherwise stated individual concentrations of such optional components may range from about 0.001 wt. % to about 20 wt. %, based on the weight of the total composition.

Non-limiting examples of optional components for use in the composition include insoluble or particulate materials, conditioning agents (silicones, hydrocarbon oils, fatty esters), auxiliary viscosity modifiers, humectants, sensates, botanicals, amino acids, vitamins, chelating agents, buffering agents, pH adjusting agents, preservatives perfumes and fragrances, electrolytes, dyes and pigments, nonvolatile solvents or diluents (water soluble and insoluble), foam boosters, sunscreens and UV absorbers.

### 1. Insoluble and Particulate Materials

In the compositions of the present technology, the nonionic, amphiphilic emulsion polymers of the disclosed technology can be utilized to enhance foaming properties, improve mildness and the rheology properties of cleansing compositions for the hair, scalp and skin, and can be utilized for the stable suspension of insoluble silicones, opacifiers and pearlescent agents (e.g., mica, coated mica, ethylene glycol monostearate (EGMS), ethylene glycol distearate (EGDS), polyethylene glycol monostearate (PGMS) or polyethyleneglycol distearate (PGDS)), pigments, exfoliants, auxiliary anti-dandruff agents, clay, swellable clay, laponite, gas bubbles, liposomes, microsponges, cosmetic beads, cosmetic microcapsules, and flakes, and are discussed in more detail below.

Exemplary cosmetic bead components include, but are not limited to, agar beads, alginate beads, jojoba beads, gelatin beads, Styrofoam™ beads, polyacrylate, polymethylmethacrylate (PMMA), polyethylene beads, Unispheres™ and Unipearls™ cosmetic beads (Induchem USA, Inc., New York, N.Y.), Lipocapsule™, Liposphere™, and Lipopearl™ microcapsules (Lipo Technologies Inc., Vandalia, Ohio), and Confetti II™ dermal delivery flakes (United-Guardian, Inc., Hauppauge, N.Y.). Beads can be utilized as aesthetic materials or can be used to encapsulate benefit agents to protect them from the deteriorating effects of the environment or for optimal delivery, release and performance in the final product.

In one aspect, the cosmetic beads range in size from about 0.5 to about 1.5 mm. In another aspect, the difference in specific gravity of the bead and water is between about +/−0.01 and 0.5 in one aspect, and from about +/−0.2 to 0.3 g/ml in another aspect.

In one aspect, the microcapsules range in size from about 0.5 to about 300 μm. In another aspect, the difference in specific gravity between the microcapsules and water is from about +/−0.01 to 0.5. Non-limiting examples of microcapsule beads are disclosed in U.S. Pat. No. 7,786,027, the disclosure of which is herein incorporated by reference.

### 2. Conditioning Agents

Conditioning agents include any material which is used to give a particular conditioning benefit to hair, scalp and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present technology typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

### Silicones

The silicone conditioning agent may comprise volatile silicones, non-volatile silicones, and mixtures thereof. If volatile silicones are present, they are typically employed as a solvent or carrier for commercially available forms of non-volatile silicone fluid conditioning agents such as oils and gums and resins. Volatile silicone fluids are often included in the conditioning package to improve silicone fluid deposition efficacy or to enhance the shine, sheen or glossiness of the hair. Volatile silicone materials are frequently included in formulations to enhance sensory attributes (e.g., feel) on the hair, scalp and skin

In one aspect, the silicone conditioning agent is non-volatile and includes silicone oils, gums, resins and mixtures thereof. By non-volatile is meant that the silicone has a very low vapor pressure at ambient temperature conditions (e.g., less than 2 mm Hg at 20° C.). The non-volatile silicone conditioning agent has a boiling point above about 250° C. in one aspect, above about 260° C. in another aspect, and above about 275° C. in a further aspect. Background information on silicones including sections discussing silicone oils, gums, and resins, as well as their manufacture, are found in Encyclopedia of Polymer Science and Engineering, vol. 15, 2d ed., pp 204-308, John Wiley & Sons, Inc. (1989).

### Silicone Oil

In one aspect, the silicone conditioning agent is silicone oil selected from a polyorganosiloxane material. In one aspect, the polyorganosiloxane material can be selected from polyalkylsiloxanes, polyarylsiloxanes, polyalkylarylsiloxanes, hydroxyl terminated polyalkylsiloxanes, polyarylalkylsiloxanes, amino functional polyalkylsiloxanes, quaternary functional polyalkylsiloxanes, and mixtures thereof.

In one aspect, the silicone oil conditioning agent includes polyorganosiloxanes represented by the formula:

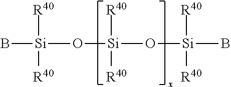

wherein B independently represents hydroxy, methyl, methoxy, ethoxy, propoxy, and phenoxy; R⁴⁰ independently represents methyl, ethyl, propyl, phenyl, methylphenyl, phenylmethyl, a primary, secondary or tertiary amine, a quaternary group selected from a group selected from:
—R⁴¹—N(R⁴²)CH₂CH₂N(R⁴²)₂;
—R⁴¹—N(R⁴²)₂;
—R⁴¹—N+(R⁴²)₃CA⁻; and
—R⁴¹—N(R⁴²)CH₂CH₂N⁺(R⁴²)H₂ CA⁻;
   wherein R⁴¹ is a linear or branched, hydroxyl substituted or unsubstituted alkylene or alkylene ether moiety containing 2 to 10 carbon atoms; R⁴² is hydrogen, C₁-C₂₀ alkyl (e.g, methyl), phenyl or benzyl; q is an integer ranging from about 2 to about 8; CA⁻ is a halide ion selected from chlorine, bromine, iodine and fluorine; and x is an integer ranging from about 7 to about 8000 in one aspect, from about 50 to about 5000 in another aspect, form about 100 to about 3000 in still another aspect, and from about 200 to about 1000 in a further aspect.

In one aspect, the amino functional polyalkylsiloxane can be represented by the formula:

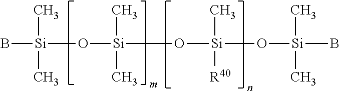

wherein B independently represents hydroxy, methyl, methoxy, ethoxy, propoxy, and phenoxy; and R⁴⁰ is selected from:
—R⁴¹—N(R⁴²)CH₂CH₂N(R⁴²)₂;
—R⁴¹—N(R⁴²)₂;
—R⁴¹—N+(R⁴²)₃CA⁻; and
—R⁴¹—N(R⁴²)CH₂CH₂N⁺(R⁴²)H₂ CA⁻
   wherein R⁴¹ is a linear or branched, hydroxyl substituted or unsubstituted alkylene or alkylene ether moiety containing 2 to 10 carbon atoms; R⁴² is hydrogen, C₁-C₂₀ alkyl (e.g, methyl), phenyl or benzyl; CA⁻ is a halide ion selected from chlorine, bromine, iodine and fluorine; and the sum of m+n ranges from about 7 to about 1000 in one aspect, from about 50 to about 250 in another aspect, and from about 100 to about 200 in another aspect, subject to the proviso that m or n is not 0. In one aspect B is hydroxy and R⁴⁰ is —(CH₂)₃NH(CH₂)₃NH₂. In another aspect B is methyl and R⁴⁰ is —(CH₂)₃NH(CH₂)₃NH₂. In still another aspect B is methyl and R⁴⁰ is a quaternary ammonium moiety represented by —(CH₂)₃OCH₂CH(OH)CH₂N(R⁴²)₃ CA⁻; wherein R⁴² and CA⁻ are as previously defined.

The silicone oil conditioning agents can have a viscosity ranging from about above about 25 to about 1,000,000 mPa·s at 25° C. in one aspect, from about 100 to about 600,000 mPa·s in another aspect, and from about 1000 to about 100,000 mPa·s still another aspect, from about 2,000 to about 50,000 mPa·s in yet another aspect, and from about 4,000 to about 40,000 mPa·s in a further aspect. The viscosity is measured by means of a glass capillary viscometer as described by Dow Corning Corporate Test Method CTM004, dated Jul. 20, 1970. In one aspect the silicone oils have an average molecular weight below about 200,000 daltons. The average molecular weight can typically range from about 400 to about 199,000 daltons in one aspect, from about 500 to about 150,000 daltons in another aspect, from about 1,000 to about 100,000 daltons in still another aspect, from about 5,000 to about 65,000 daltons in a further aspect.

Exemplary silicone oil conditioning agents include, but are not limited to, polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxanes (amodimethicones), and mixtures thereof.

### Silicone Gum

Another silicone conditioning agent useful in the disclosed technology is a silicone gum. A silicone gum is a polyorganosiloxane material of the same general structure of the silicone oils set forth above wherein B independently represents hydroxy, methyl, methoxy, ethoxy, propoxy, and phenoxy; R⁴⁰ independently represents methyl, ethyl, propyl, phenyl, methylphenyl, phenylmethyl, and vinyl. Silicone gums have a viscosity measured at 25° C. of greater than 1,000,000 mPa·s. The viscosity can be measured by means of a glass capillary viscometer as described above for the silicone oils. In one aspect the silicone gums have an average molecular weight about 200,000 daltons and above. The molecular weight can typically range from about 200,000 to about 1,000,000 daltons. It is recognized that the silicone gums described herein can also have some overlap with the silicone oils described previously. This overlap is not intended as a limitation on any of these materials.

Suitable silicone gums for use in the silicone component of compositions of the disclosed technology are polydimethylsiloxanes (dimethicones), optionally having terminal end groups such as hydroxyl (dimethiconols), polymethylvinylsiloxane, polydiphenylsiloxane, and mixtures thereof.

### Silicone Resins

Silicone resins can be included as a silicone conditioning agent suitable for use in the compositions of the disclosed technology. These resins are crosslinked polysiloxanes. The crosslinking is introduced through the incorporation of trifunctional and tetrafunctional silanes with monofunctional and/or difunctional silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetra-functional siloxane monomer units (and hence, a sufficient level of crosslinking) such that they form a rigid or hard film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. In one aspect, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and terachlorosilane, with the methyl substituted silanes being most commonly utilized.

Silicone materials and silicone resins can be identified according to a shorthand nomenclature system known to those of ordinary skill in the art as “MDTQ” nomenclature. Under this naming system, the silicone is described according to the presence of various siloxane monomer units which make up the silicone. The “MDTQ” nomenclature system is described in the publication entitled “Silicones: Preparation, Properties and Performance”; Dow Corning Corporation, 2005, and in U.S. Pat. No. 6,200,554.

Exemplary silicone resins for use in the compositions of the disclosed technology include, but are not limited to MQ, MT, MTQ, MDT and MDTQ resins. In one aspect, methyl is the silicone resin substituent. In another aspect, the silicone resin is selected from a MQ resins, wherein the M:Q ratio is from about 0.5:1.0 to about 1.5:1.0 and the average molecular weight of the silicone resin is from about 1000 to about 10,000 daltons.

### Volatile Silicones

The optional volatile silicones referred to above include linear polydimethylsiloxanes and cyclic polydimethylsiloxanes (cyclomethicones), and mixtures thereof. Volatile linear polydimethylsiloxanes (dimethicones) typically contain about 2 to about 9 silicon atoms, alternating with oxygen atoms in a linear arrangement. Each silicon atom is also substituted with two alkyl groups (the terminal silicon atoms are substituted with three alkyl groups), such as, for example, methyl groups. The cyclomethicones typically contain about 3 to about 7 dimethyl substituted silicon atoms in one aspect and from about 3 to about 5 in another aspect, alternating with oxygen atoms, in a cyclic ring structure. The term “volatile” means that the silicone has a measurable vapor pressure, or a vapor pressure of at least 2 mm of Hg at 20° C. The volatile silicones have a viscosity of 25 mPa·s or less at 25° C. in one aspect, from about 0.65 about to about 10 mPa·s in another aspect, from about 1 to about 5 mPa·s in still another aspect, and from about 1.5 to about 3.5 mPa·s in a further aspect. A description of linear and cyclic volatile silicones is found in Todd and Byers, “Volatile Silicone Fluids for Cosmetics”, Cosmetics and Toiletries, Vol. 91(1), pp. 27-32 (1976), and in Kasprzak, “Volatile Silicones”, Soap/Cosmetics/Chemical Specialities, pp. 40-43 (December 1986).

Exemplary volatile linear dimethicones include, but are not limited to, hexamethyldisiloxane, octamethyltrisiloxane, decamethyltetrasiloxane, dodecamethylpentasiloxane and blends thereof. Volatile linear dimethicones and dimethicone blends are commercially available from Dow Corning Corporation as Dow Corning 200® Fluid (e.g., product designations 0.65 CST, 1 CST, 1.5 CST, and 2 CST) and Dow Corning® 2-1184 Fluid.

Exemplary volatile cyclomethicones are D4 cyclomethicone (octamethylcyclotetrasiloxane), D5 cyclomethicone (decamethylcyclopentasiloxane), D6 cyclomethicone, and blends thereof (e.g., D4/D5 and D5/D6). Volatile cyclomethicones and cyclomethicone blends are commercially available from Momentive Performance Materials Inc. as SF1173, SF1202, SF1256, and SF1258 silicone fluids, and Dow Corning Corporation as Dow Corning® 244, 245, 246, 345, and 1401 silicone fluids. Blends of volatile cyclomethicones and volatile linear dimethicones also can be employed.

The amount of silicone conditioner(s) in the compositions of the present technology should be sufficient to provide the desired conditioning performance to the hair, and generally ranges from about 0.01 to about 20 wt. % in one aspect, from about 0.05 to about 15 wt. % in another aspect, from about 0.1% to about 10 wt. % in still another aspect, and from about 1 to about 5 wt. % in a further aspect, based on the total weight of the composition.

### Hydrocarbon Oils

The conditioning component of the compositions of the disclosed technology can also contain hydrocarbon oil conditioners.

Suitable conditioning oils for use as conditioning agents in the compositions of the disclosed technology include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils typically contain about 12 to 19 carbon atoms. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from BP Chemical Company.

Liquid polyolefin conditioning oils can be used in the hair straightening compositions of the present technology. The liquid polyolefin conditioning agents are typically poly-α-olefins that have been hydrogenated. Polyolefins for use herein can be prepared by the polymerization of C₄ to about C₁₄ olefinic monomers. Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. In one aspect of the disclosed technology, hydrogenated α-olefin monomers include, but are not limited to: 1-hexene to 1-hexadecenes, 1-octene to 1-tetradecene, and mixtures thereof.

Fluorinated or perfluorinated oils are also contemplated within the scope of the present technology. Fluorinated oils include perfluoropolyethers described in European Patent 0 486 135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

### Natural Oils

Natural oil conditioners are also useful in the practice of the disclosed technology and include but are not limited to peanut, sesame, avocado, coconut, cocoa butter, almond, safflower, corn, cotton seed, sesame seed, walnut oil, castor, olive, jojoba, palm, palm kernel, soybean, wheat germ, linseed, sunflower seed; eucalyptus, lavender, vetiver, litsea, cubeba, lemon, sandalwood, rosemary, chamomile, savory, nutmeg, cinnamon, hyssop, caraway, orange, geranium, cade, and bergamot oils, fish oils, glycerol tricaprocaprylate; and mixtures thereof.

### Ester Oils

Ester oil conditioners include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.).

Exemplary fatty esters include, but are not limited to isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the disclosed technology are mono-carboxylic acid esters of the general formula R⁶⁰C(O)OR⁶¹, wherein R⁶⁰ and R⁶¹ are alkyl or alkenyl radicals, and the sum of carbon atoms in R⁶⁰ and R⁶¹ is at least 10 in one aspect, and at least 22 in another aspect of the disclosed technology.

Still other fatty esters suitable for use in the compositions of the disclosed technology are di- and tri-alkyl and alkenyl esters of carboxylic acids, such as esters of C₄ to C₈ dicarboxylic acids (e.g., C₁ to C₂₂ esters, preferably C₁ to C₆, of succinic acid, glutaric acid, adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the disclosed technology are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Specific non-limiting examples of suitable synthetic fatty esters include: P-43 (C₈ to C₁₀ triester of trimethylolpropane), MCP-684 (tetraester of 3,3 diethanol-1,5 pentadiol), MCP 121 (C₈ to C₁₀ diester of adipic acid), all of which are available from ExxonMobil Chemical Company.

The amount of hydrocarbon and natural conditioning oils and ester oil conditioning agents can range from about 0.05 to about 10 wt. %, in one aspect, from about 0.5 to about 5 wt. % in another aspect, and from about 1 to about 3 wt. % in a further aspect, based on the total weight of the composition.

### Cationic Compounds and Polymers

Cationic Compounds refer to non-polymeric and polymeric compounds containing at least one cationic moiety or at least one moiety that can be ionized to form a cationic moiety. Typically these cationic moieties are nitrogen containing groups such as quaternary ammonium or protonated amino groups. The cationic protonated amines can be primary, secondary, or tertiary amines. In one aspect, the cationic conditioning compounds include quaternary nitrogen containing non-polymeric and polymeric materials that well known in the art for hair conditioning. Cationic conditioning compounds include non-polymeric compounds containing one quaternary ammonium salt moiety and polymeric compounds (polymers) containing at least one quaternary ammonium salt moiety.

In one aspect, the quaternary ammonium salt moiety corresponds to the general formula: (R⁷⁰)(R⁷¹)(R⁷²)(R⁷³)N⁺) E⁻ where each of R⁷⁰, R⁷¹, R⁷⁴, and R⁷⁵ are independently selected from an aliphatic group having from 1 to about 22 carbon atoms (e.g., alkyl, alkenyl); an aromatic (e.g., phenyl benzyl); alkoxy; polyoxyalkylene (e.g., polyethylene, polypropylene, and combinations thereof); acetamido; alkylamido; alkylamidoalkyl; hydroxyalkyl; aryl; araalkyl; or alkylaryl group having 1 to about 22 carbon atoms in the alkyl chain; and E⁻ is a salt-forming anion such as those selected from halogen, (e.g., chloride, bromide), acetate, citrate, lactate, glycolate, phosphate, nitrate, sulfate, and alkylsulfate (e.g., methosulfate, ethosulfate). The aliphatic groups can contain, in addition to carbon and hydrogen atoms, ether linkages, ester linkages, and other groups such as amino groups. The longer chain aliphatic groups, e.g., those of about 12 carbons, or higher, can be saturated or unsaturated. Any two of R⁷⁰, R⁷¹, R⁷⁴, and R⁷⁵ together with the nitrogen atom to which they are attached can be taken together to form a ring structure containing 5 to 6 carbon atoms, one of said carbon atoms can optionally be replaced with a heteroatom selected from nitrogen, oxygen or sulfur.

In one aspect, the quaternary ammonium moiety contains at least one nitrogen atom that is covalently linked to at least three alkyl and/or aryl substituents, and the nitrogen atom remains positively charged regardless of the environmental pH.

In one aspect, the quaternary ammonium moiety contains one nitrogen atom and at least one C₁₂ to C₂₂ alkyl group. In one aspect, the quaternary ammonium moiety contains one C₁₂ to C₂₂ alkyl group and at least two C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and three C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and two C₁ to C₅ alkyl groups (e.g., methyl, ethyl, propyl, butyl and pentyl, and combinations thereof), and one moiety containing an alkoxy; polyoxyalkylene (e.g., polyethylene, polypropylene, and combinations thereof), where the polyoxyalkylene moiety contains 3 to 100 repeating units; acetamide; alkylamido; alkylamidoalkyl; hydroxyalkyl; aryl; araalkyl; or alkylaryl group having 1 to about 22 carbon atoms in the alkyl chain, and having 6 to about 14 carbon atoms in the aryl moiety.

A number of quaternary nitrogen-containing compounds and polymers, their manufacturers and general descriptions of their chemical characteristics are found in the CTFA Dictionary and in the International Cosmetic Ingredient Dictionary, Vol. 1 and 2, 5th Ed., published by the Cosmetic Toiletry and Fragrance Association, Inc. (CTFA) (1993), the pertinent disclosures of which are incorporated herein by reference. The name assigned to the ingredients by the CTFA or by the manufacturer is used for convenience.

Non-limiting examples of monomeric quaternary ammonium compounds useful as cationic conditioners in the present technology include Acetamidopropyl Trimonium Chloride, Behenamidopropyl Ethyldimonium Ethosulfate, Behentrimonium Chloride, Behentrimonium Methosulfate, Cetethyl Morpholinium Ethosulfate, Cetrimonium Chloride, Cocoamidopropyl Ethyldimonium Ethosulfate, Dicetyldimonium Chloride, Hydroxyethyl Behenamidopropyl Dimonium Chloride, Quaternium-26, Quaternium-27, Quaternium-53, Quaternium-63, Quaternium-70, Quaternium-72, Quaternium-76 PPG-9 Diethylmonium Chloride, PPG-25 Diethylmonium Chloride, PPG-40 Stearalkonium Chloride, Isostearamidopropyl Ethyldimonium Ethosulfate, and mixtures thereof.

Cationic polymers are also useful as conditioning agents alone or in combination with the other conditioning agents described herein. Suitable cationic polymers can be synthetically derived or natural polymers can be synthetically modified to contain cationic moieties. Polymeric quaternary ammonium moiety salt containing polymers can be prepared by the polymerization of a diallylamine such as dialkyldiallylammonium salt or copolymer thereof in which the alkyl group contains 1 to about 22 carbon atoms in one aspect and methyl or ethyl in another aspect. Copolymers containing a quaternary moiety derived from a dialkyldiallylammonium salt and an anionic component derived from anionic monomers of acrylic acid and methacrylic acid are suitable conditioning agents. Also suitable are, polyampholyte terpolymers having a cationic component prepared from a derivative of diallylamine, such as a dimethyldiallylammonium salt, an anionic component derived from anionic monomers of acrylic acid or 2-acrylamido-2-methylpropane sulfonic acid and a nonionic component derived from nonionic monomers of acrylamide. The preparation of such quaternary ammonium salt moiety containing polymers can be found, for example, in U.S. Pat. Nos. 3,288,770; 3,412,019; 4,772,462 and 5,275,809, the pertinent disclosures of which are incorporated herein by reference.

In one aspect, suitable cationic polymers include the chloride salts of the foregoing quaternized homopolymers and copolymers in which the alkyl group is methyl or ethyl, and are commercially available under the Merquat® series of trademarks from Lubrizol Advanced Materials, Inc.

A homopolymer prepared from diallyl dimethyl ammonium chloride (DADMAC) having the CTFA name, Polyquaternium-6, is available under the Merquat 100 and Merquat 106 trademark. A copolymer prepared from DADMAC and acrylamide having the CTFA name, Polyquaternium-7, is sold under the Merquat 550 trademark. Another copolymer prepared from DADMAC and acrylic acid having the CTFA name, Polyquaternium-22, is sold under the Merquat 280 trademark. The preparation of Polyquaternium-22 and its related polymers is described in U.S. Pat. No. 4,772,462, the pertinent disclosures of which are incorporated herein by reference.

Also useful is an ampholytic terpolymer prepared from a nonionic component derived from acrylamide or methyl acrylate, a cationic component derived from DADMAC or methacrylamidopropyl trimethyl ammonium chloride (MAPTAC), and an anionic component derived from acrylic acid or 2-acrylamido-2-methylpropane sulfonic acid or combinations of acrylic acid and 2-acrylamido-2-methylpropane sulfonic acid. An ampholytic terpolymer prepared from acrylic acid, DADMAC and acrylamide having the CTFA name, Polyquarternium-39, is available under the Merquat Plus 3330 trademark. Another ampholytic terpolymer prepared from acrylic acid, methacrylamidopropyl trimethyl ammonium chloride (MAPTAC) and methyl acrylate having the CTFA name, Polyquarternium-47, is available under the Merquat 2001 trademark. Still another ampholytic terpolymer prepared from acrylic acid, MAPTAC and acrylamide having the CTFA name, Polyquarternium-53, is available under the Merquat 2003PR trademark. The preparation of such terpolymers is described in U.S. Pat. No. 5,275,809, the pertinent disclosures of which are incorporated herein by reference.

Other cationic polymers and copolymers suitable as conditioners in the hair straightening compositions of the disclosed technology have the CTFA names Polyquaternium-4, Polyquaternium-11, Polyquarternium-16, Polyquaternium-28, Polyquaternium-29, Polyquaternium-32, Polyquaternium-33, Polyquaternium-35, Polyquaternium-37, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquaternium-87, and PEG-2-cocomonium chloride.

Exemplary cationically modified natural polymers suitable for use in the hair straightening composition include polysaccharide polymers, such as cationically modified cellulose and cationically modified starch derivatives modified with a quaternary ammonium halide moiety. Exemplary cationically modified cellulose polymers are salts of hydroxyethyl cellulose reacted with trimethyl ammonium substituted epoxide (CTFA, Polyquaternium-10). Other suitable types of cationically modified cellulose include the polymeric quaternary ammonium salts of hydroxyethyl cellulose reacted with lauryl dimethyl ammonium substituted epoxide (CTFA, Polyquaternium-24). Cationically modified potato starch having the CTFA name, Starch Hydroxypropyltrimonium Chloride, is available under the Sensomer™ CI-50 trademark, from Lubrizol Advanced Materials, Inc.

Other suitable cationically modified natural polymers include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc.

The non-polymeric and polymeric cationic compounds can be present from about 0.05 to about 5 wt. % percent in one aspect, from about 0.1 to about 3 wt. percent in another aspect, and from about 0.5 to about 2.0 wt. % in a further aspect (based on the total weight of the composition).

### Auxiliary Viscosity Modifier

The composition of the disclosed technology must be easily pourable with a shear thinning index of less than 0.5 at shear rates between 0.1 and 1 reciprocal second, and an optical transmission of at least 10%. The suspension agent of the disclosed technology optionally can be utilized in combination with an auxiliary rheology modifier (thickener) to enhance the yield value of a thickened liquid. In one aspect, the nonionic, amphiphilic emulsion, emulsion polymer of the disclosed technology can be combined with a nonionic rheology modifier to enhance the yield stress value of a composition in which it is included. Any rheology modifier is suitable, so long as such is soluble in water, stable and contains no ionic or ionizable groups. Suitable rheology modifiers include, but are not limited to natural gums (e.g., polygalactomannan gums selected from fenugreek, cassia, locust bean, tara and guar), modified cellulose (e.g., ethylhexylethylcellulose (EHEC), hydroxybutylmethylcellulose (HBMC), hydroxyethylmethylcellulose (HEMC), hydroxypropylmethylcellulose (HPMC), methyl cellulose (MC), hydroxyethylcellulose (HEC), hydroxypropylcellulose (HPC) and cetyl hydroxyethylcellulose); and mixtures thereof methylcellulose, polyethylene glycols (e.g., PEG 4000, PEG 6000, PEG 8000, PEG 10000, PEG 20000), polyvinyl alcohol, polyacrylamides (homopolymers and copolymers), and hydrophobically modified ethoxylated urethanes (HEUR). The rheology modifier can be utilized in an amount ranging from about 0.5 to about 25 wt. % in one aspect, from about 1 to about 15 wt. % in another aspect, and from about 2 to about 10 wt. % in a further aspect, based on the weight of the total weight of the composition.

### Humectants

Humectants are defined as materials that absorb or release water vapor, depending on the relative humidity of the environment, (Harry's Cosmeticology, Chemical Publishing Company Inc., 1982 p. 266). Suitable humectants that include, but are not limited to, allantoin; pyrrolidonecarboxylic acid and its salts; hyaluronic acid and salts thereof; sorbic acid and salts thereof; urea, lysine, cystine, and amino acids; polyhydroxy alcohols such as glycerin, propylene glycol, hexylene glycol, hexanetriol, ethoxydiglycol, dimethicone copolyol, and sorbitol, and the esters thereof; polyethylene glycol; glycolic acid and glycolate salts (e.g. ammonium and quaternary alkyl ammonium); chitosan; aloe-vera extracts; algae extract; honey and derivatives thereof; inositol; lactic acid and lactate salts (e.g. ammonium and quaternary alkyl ammonium); sugars and starches (e.g., maltose, glucose, fructose); sugar and starch derivatives (e.g., glucose alkoxylated glucose, mannitol, xyliyol); DL-panthenol; magnesium ascorbyl phosphate, arbutin, kojic acid, lactamide monoethanolamine; acetamide monoethanolamine; and the like, and mixtures thereof. Humectants also include the C₃ to C₆ diols and triols, such as glycerin, propylene glycol, butane-1,2,3-triol, hexylene glycol, hexanetriol, and the like, and mixtures thereof. Ethoxylated methyl glucose ethers containing an average of 5 to 30 moles of ethoxylation, such as, for example, those available under the INCI names Lauryl Methyl Gluceth-10 Hydroxypropyldimonium chloride, Methyl Gluceth-10 and Methyl Gluceth-20, are suitable.

Such humectants may be present at from 0.01-20 wt. % of the composition, such as at least 0.1 wt. %, or at least 1 wt. %, e.g., up to 8 wt. %, or up to 5 wt. %.

### Sensates

A skin sensate helps provide a sensory confirmation of the adequacy, activity and evenness of the application thereof by a user. Some non-limiting examples of skin sensates are described in U.S. Pat. Nos. 4,230,688, 4,136,163, 6,183,766 and 7,001,594 each of which are incorporated herein by reference in their entireties. Non-limiting examples of suitable sensates include butanedioic acid monomenthyl ester, camphor, carvone, cineole, clove oil, ethyl carboxamide, ethyl menthane carboxamide, eucalyptus oil, eucolytol, ginger oil, I-isopulegol, menthol, menthone glycerin acetal, menthoxy-1,2-propanediol, menthyl lactate, methyl diisopropylpropioniamide, methyl salicylate, peppermint oil, rosemary oil, trimethyl butanamide, vanillyl butyl ether or combinations thereof. The sensate can be included in the composition in amounts ranging from about 0.01 wt. % to about 2 wt. % in one aspect, and from about 0.05 wt. % to about 1 wt. % in another aspect, based on the total weight of the composition. Botanicals

The hair care compositions of the disclosed technology can contain one or more botanical agents. Suitable botanical agents can include, for example, extracts from Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, apricot kernel, sage, strawberry, sweet pea, tomato, sunflower seed extract, sandalwood extract, grape seed, aloe leaf, vanilla fruit, comfrey, arnica, Centella asiatica, cornflower, horse chestnut, ivy, Macadamia ternifolia seed, magnolia, oat, pansy, skullcap, seabuckthorn, white nettle, and witch hazel. Botanical extracts may also include, for example, chlorogenic acid, glutathione, glycrrhizin, neohesperidin, quercetin, rutin, morin, myricetin, absinthe, and chamomile.

In one aspect, the hair care composition can contain from about 0.01 wt. % to about 10 wt. % of one or more of the botanical extracts set forth above, from about 0.05 wt. % to about to about 5 wt. % in another aspect, from about 0.1 wt. % to about 3 wt. % in still another aspect, and from about 0.5 wt. % to about 1 wt. % in a further aspect, based on the total weight of the composition. Amino Acids

The hair care composition provided herein can contain one or more non-guanidine moiety containing amino acids. Examples of amino acids that can be used include, without limitation, capryl keratin amino acids, capryl silk amino acids, jojoba amino acids, keratin amino acids, palmitoyl keratin amino acids, palmitoyl silk amino acids, sodium cocoyl amino acids, sodium cocoyl silk amino acids, and sweet almond amino acids.

The hair straightening composition can include an appropriate amount of amino acid(s). The amount of amino acid ranges from about 0.001 wt. % to about 5 wt. % in one aspect, from about 0.01 wt. % percent to about 3 wt. % in another aspect, from about 0.1 wt. % to about 2 wt. % in still another aspect, and from about 0.5 wt. % to about 1 wt. % in a further aspect, based on the total weight of the composition.

### Vitamins

The hair care composition can contain one or more vitamins. Examples of vitamins that can be used include, without limitation, niacinamide, sodium starch octenylsuccinate, calcium pantothenate, maltodextrin, sodium ascorbyl phosphate, tocopheryl acetate, pyridoxine HCl, silica, panthenol (e.g., Pro Vitamin B5), phytantriol, calcium pantothenate (e.g., vitamin B5), vitamin E, and vitamin E esters (e.g., tocopheryl acetate, tocopheryl nocotinate, tocopheryl palmitate, or tocopheryl retinoate).

A hair care composition provided herein can include any amount of vitamin(s). The amount of vitamin(s) can range from about 0.05 wt. % to about 10 wt. % in one aspect, from about 0.1 wt. % to about 5 wt. % in another aspect, from about 0.5 wt. % to about 3 wt. % in still another aspect, and from about 0.75 wt. % to about 1 wt. % in a further aspect, based on the total weight of the composition.

### Chelating Agents

Chelating agents can be employed to stabilize the composition against the deleterious effects of metal ions. When utilized, suitable chelating agents include EDTA (ethylene diamine tetraacetic acid) and salts thereof such as disodium EDTA, citric acid and salts thereof, cyclodextrins, and the like, and mixtures thereof.

Such suitable chelating agents can comprise 0.001 wt. % to 3 wt. %, such as 0.01 wt. % to 2 wt. %, or 0.01 wt. % to 1 wt. % of the total weight of the hair straightening composition.

### Buffer Agents

Buffering agents can be used in the exemplary compositions. Suitable buffering agents include alkali or alkali earth metal carbonates, phosphates, bicarbonates, citrates, borates, acetates, acid anhydrides, succinates, and the like, such as sodium phosphate, sodium citrate, sodium acetate, sodium bicarbonate, and sodium carbonate.

### pH Adjusting Agents

The pH of the composition can range from to 1.5 to 9.5 in one aspect, at least 4.5 in a second aspect, at least 5.5 a third aspect, at least 6.5 in a fourth aspect, at least 7.0 in a fifth aspect, at least 7.5 in a sixth aspect, at least 8.0 in a seventh aspect, at least 8.5 in an eighth aspect, at least 9.0 in a ninth aspect, and at least 9.5 in a tenth aspect.

When polyvalent metal salts of pyrithione in combination with secondary zinc salts are employed in the antidandruff hair care compositions of the disclosed technology, the pH of the composition is adjusted to a value of at least about 6.5. The pH can range from about 6.5 to about 12 in one aspect, from about 6.8 to about 9.5 in another aspect, and from about 6.8 to about 8.5 in still another aspect. To provide the desired pH, the composition may be adjusted with one or more pH modifiers selected from organic and inorganic acids and bases.

The pH of the composition can be adjusted with any combination of acidic and/or basic pH adjusting agents known to the art. Acidic materials include organic acids and inorganic acids, in particular, monocarboxylic acids, dicarboxylic acids, and tricarboxylic acids, for example, acetic acid, citric acid, tartaric acid, alpha-hydroxy acids, beta-hydroxy acids, salicylic acid, lactic acid, malic acid, glycolic acid, amino acids, and natural fruit acids, or inorganic acids, for example, hydrochloric acid, nitric acid, sulfuric acid, sulfamic acid, phosphoric acid, and combinations thereof.

Basic materials include inorganic and organic bases, and combinations thereof. Examples of inorganic bases include but are not limited to the alkali metal hydroxides (e.g., potassium hydroxide, sodium hydroxide) and alkali metal carbonates (e.g., potassium carbonate, sodium carbonate), and alkali metal salts such as sodium borate (borax), sodium phosphate, sodium pyrophosphate, and the like; and mixtures thereof. Examples of organic bases include ammonium hydroxide, triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), and PEG-15 cocamine.

The pH adjusting agent(s) and/or buffering agent is utilized in any amount necessary to obtain and/or maintain a desired pH value in the composition.

### Preservatives

In one aspect, any preservative suitable for use in personal care can be used in the composition for straightening hair. Suitable preservatives include polymethoxy bicyclic oxazolidine, methyl paraben, propyl paraben, ethyl paraben, butyl paraben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, and suitable polyquaternium compounds as disclosed above (e.g., Polyquaternium-1).

In another aspect, acid based preservatives are useful in the exemplary compositions. The use of acid based preservatives facilitates the formulation of products in the low pH range. Lowering the pH of a formulation inherently provides an inhospitable environment for microbial growth in addition to being suited to the straightening process. Moreover, formulating at low pH enhances the efficacy of acid based preservatives, and affords a personal care product which maintains an acidic pH balance on the skin. Any acid based preservative that is useful in personal care products can be used in the exemplary compositions. In one aspect the acid preservative is a carboxylic acid compound represented by the formula: R⁸⁰C(O)OH, wherein R⁸⁰ represents hydrogen, a saturated and unsaturated hydrocarbyl group containing 1 to 8 carbon atoms or C₆ to C₁₀ aryl. In another aspect, R⁸⁰ is selected from a hydrogen, a C₁ to C₈ alkyl group, a C₂ to C₈ alkenyl group, or phenyl. Exemplary acids are, but are not limited to, formic acid, acetic acid, propionic acid, sorbic acid, caprylic acid, and benzoic acid, and mixtures thereof.

In another aspect, suitable acids include but are not limited to, oxalic acid, succinic acid, glutaric acid, adipic acid, azelaic acid, maleic acid, fumaric acid, lactic acid, glyceric acid, tartronic acid malic acid, tartaric acid, gluconic acid, citric acid, ascorbic acid, salicylic acid, phthalic acid, mandelic acid, benzilic acid, and mixtures thereof.

Salts of the foregoing acids are also useful as long as they retain efficacy at low pH values. Suitable salts include the alkali metal (e.g., sodium, potassium, calcium) and ammonium salts of the acids enumerated above.

The acid based preservatives and/or their salts can be used alone or in combination with non-acidic preservatives typically employed in personal care, home care, health care, and institutional and industrial care products.

The preservatives may comprise from 0.01 wt. % to 3.0 wt. % in one aspect, or from about 0.1 wt. % to about 1 wt. %, or from about 0.3 wt. % to about 1 wt. %, of the total weight of the hair care composition.

### Perfumes and Fragrances

Fragrance and perfume components that may be used in the exemplary composition to mask the odor of any of the various components in the hair straightening composition or to give the composition an aesthetically pleasing fragrance. In one aspect, suitable fragrances and perfumes include natural and synthetic fragrances, perfumes, scents, and essences and any other substances which emit a fragrance. As the natural fragrances, there are those of vegetable origin, such as oil extracts from flowers (e.g., lily, lavender, rose, jasmine, neroli, ylang-ylang), stems and leaves (geranium, patchouli, petitgrain, peppermint), fruits (aniseed, coriander, fennel, mace, needle juniper), fruit skin (bergamot, lemon, orange), roots (angelica, celery, cardamom, costus, iris, sweet flag), woods (pine tree, sandalwood, guaiacum wood, cedar, rosewood, cinnamon), herbs and grasses (tarragon, lemongrass, sage, thyme), needles and twigs (spruce, pine, European red pine, stone pine), and resins and balsam (galbanum, elemi, benzoin, myrrh, frankincense, opopanax), and those of animal origin, such as musk, civet, castoreum, ambergris, or the like, and mixtures thereof.

Examples of synthetic fragrances and perfumes are the aromatic esters, ethers, aldehydes, ketones, alcohols, and hydrocarbons including benzyl acetate, phenoxyethyl isobutylate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styralyl propionate, and benzyl salicylate; benzylethyl ether; straight chain alkanals having 8 to 18 carbon atoms, citral, citronellal, citronellyloxyaldehyde, cyclamen aldehyde, hydroxycitronellal, lilial, and bougeonal; ionone compounds, a-isomethyl ionone, and methyl cedryl ketone; anethole, citronellol, eugenol, isoeugenol, geraniol, lavandulol, nerolidol, linalool, phenylethyl alcohol, and terpineol, alpha-pinene, terpenes (e.g., limonene), and balsams, and mixtures thereof.

The amount of fragrance agent or perfume employed can be any amount suitable to mask a particular odor or to impart a desired aesthetically pleasing aroma, fragrance or scent. In one aspect, the amount of fragrance agent can range from about 0.05 wt. % to about 10 wt. %, from about 0.1 wt. % to about 5 wt. % in another aspect, from about 0.5 wt. % to about 3.5 wt. % in still another aspect, and from about 1 wt. % to about 2.5 wt. % in a further aspect, based on the total weight of the composition.

### Electrolytes

Optionally, the cleansing and conditioning compositions of the disclosed technology can contain an electrolyte. Suitable electrolytes are known compounds and include salts of multivalent anions, such as potassium pyrophosphate, potassium tripolyphosphate, and sodium or potassium citrate, salts of multivalent cations, including alkaline earth metal salts such as calcium chloride and calcium bromide, as well as zinc halides, barium chloride, magnesium sulfate and calcium nitrate, salts of monovalent cations with monovalent anions, including alkali metal or ammonium halides, such as potassium chloride, sodium chloride, potassium iodide, sodium bromide, and ammonium bromide, alkali metal or ammonium nitrates, and blends thereof. The amount of the electrolyte used will generally depend on the amount of the amphiphilic emulsion polymer incorporated, but may be used at concentration levels of from about 0.1 to about 4 wt. % in one aspect and from about 0.2 to about 2 wt. % in another aspect, based on the weight of the total composition.

### Dyes and Pigments

The hair care compositions of the present technology may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. and FD&C designations.

Exemplary pigments are metal compounds or semi metallic compounds and may be used in ionic, nonionic or oxidized form. The pigments can be in this form either individually or in admixture or as individual mixed oxides or mixtures thereof, including mixtures of mixed oxides and pure oxides. Examples are the titanium oxides (e.g., TiO₂), zinc oxides (e.g., ZnO), aluminum oxides (for example, Al₂O₃), iron oxides (for example, Fe₂O₃), manganese oxides (e.g., MnO), silicon oxides (e.g., SiO₂), silicates, cerium oxides, zirconium oxides (e.g., ZrO₂), barium sulfate (BaSO₄), nylon-12, and mixtures thereof.

Other examples of pigments include thermochromic dyes that change color with temperature, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide nanoparticles, barium oxide, ultramarine blue, bismuth citrate, hydroxyapatite, zirconium silicate, carbon black particles, and the like.

### Detersive Compositions

Surprisingly, the nonionic, amphiphilic emulsion polymers of the disclosed technology can be activated by a surfactant to provide a stable yield stress hair care composition with desirable rheological and aesthetic properties and the ability to suspend particulate and insoluble materials in an aqueous medium for indefinite periods of time independent of pH. The yield stress value, elastic modulus and optical clarity are substantially independent of pH in the compositions in which the present polymers are included. The nonionic, amphiphilic emulsion polymers of the disclosed technology are useful in the pH range of from about 2 to about 14 in one aspect, from about 3 to 11 in another aspect, and from about 4 to about 9 in a further aspect. Unlike the pH responsive crosslinked polymers (acid or base sensitive) that require neutralization with an acid or a base to impart a desired rheological profile, the crosslinked, nonionic, amphiphilic emulsion polymers of the disclosed technology are substantially independent of pH. By substantially independent of pH is meant that the yield stress fluid within which the polymer of the disclosed technology is included imparts a desired rheological profile (e.g., a yield stress of at least 0.1 Pa in one aspect, at least at least 0.5 Pa in another aspect, at least 1 Pa in still another aspect, and at least 2 Pa in a further aspect) across a wide pH range (e.g., from about 2 to about 14) wherein the standard deviation in yield stress values across the pH range is less than 1 Pa in one aspect, less than 0.5 Pa in another aspect, and less than 0.25 Pa in a further aspect of the.

In one exemplary aspect, the hair care compositions comprise: i) at least one nonionic, amphiphilic emulsion polymer; ii) at least one surfactant selected from at least one anionic surfactant, at least one amphoteric surfactant, at least one nonionic surfactant, and combinations thereof; iii) at least one particulate antidandruff agent; and iv) water.

In another exemplary aspect, the hair care compositions comprise: i) at least one crosslinked, nonionic, amphiphilic emulsion polymer; ii) at least one anionic surfactant; iii) at least one particulate antidandruff agent; and iv) water.

In another exemplary aspect, the hair care compositions comprise: i) at least one crosslinked, nonionic, amphiphilic emulsion polymer; ii) at least one anionic surfactant and at least one amphoteric surfactant; iii) at least one particulate antidandruff agent; and iv) water.

In another exemplary aspect, the hair care compositions comprise: i) at least one crosslinked, nonionic, amphiphilic emulsion polymer; ii) at least one anionic surfactant, iii) an optional nonionic surfactant; iv) a particulate antidandruff agent; and v) water.

In another exemplary aspect, the hair care compositions comprise: i) at least one crosslinked, nonionic, amphiphilic emulsion polymer; ii) at least one anionic surfactant, iii) an amphoteric surfactant; iv) an optional nonionic surfactant; v) a particulate antidandruff agent; and vi) water.

In another exemplary aspect, the hair care compositions comprise: i) at least one crosslinked, nonionic, amphiphilic emulsion polymer; ii) at least one anionic ethoxylated surfactant; iii) an optional nonionic surfactant; iv) a particulate antidandruff agent; and v) water. In one aspect, the average degree of ethoxylation in the anionic ethoxylated surfactant can range from about 1 to about 3. In another aspect, the average degree of ethoxylation is about 2.

In another exemplary aspect, the hair care compositions comprise: i) at least one crosslinked, nonionic, amphiphilic emulsion polymer; ii) at least one anionic ethoxylated surfactant; iii) at least one amphoteric surfactant; iv) at least one particulate antidandruff agent; v) an optional nonionic surfactant; and vi) water. In one aspect, the average degree of ethoxylation in the anionic ethoxylated surfactant can range from about 1 to about 3. In another aspect, the average degree of ethoxylation is about 2.

In another exemplary aspect, the hair care compositions comprise: i) at least one crosslinked, nonionic, amphiphilic emulsion polymer; ii) at least one anionic non-ethoxylated surfactant; iii) at least one anionic ethoxylated surfactant; iv) an optional nonionic surfactant; v) at least one particulate antidandruff agent; and vi) water. In one aspect, the average degree of ethoxylation in the anionic ethoxylated surfactant can range from about 1 to about 3. In another aspect, the average degree of ethoxylation is about 2.

In another exemplary aspect, the hair care compositions comprise: i) at least one crosslinked, nonionic, amphiphilic emulsion polymer; ii) at least one anionic non-ethoxylated surfactant; iii) at least one anionic ethoxylated surfactant; iv) at least one amphoteric surfactant; v) an optional nonionic surfactant; vi) at least one particulate antidandruff agent; and vi) water. In one aspect, the average degree of ethoxylation in the anionic ethoxylated surfactant can range from about 1 to about 3. In another aspect, the average degree of ethoxylation is about 2.

In another exemplary aspect, the hair care compositions comprise: i) at least one crosslinked, nonionic, amphiphilic emulsion polymer; ii) at least one anionic non-ethoxylated surfactant; iii) at least one anionic ethoxylated surfactant; iv) at least one amphoteric surfactant; v) an optional nonionic surfactant; vi) zinc pyrithione antidandruff agent; and vi) water. In one aspect, the average degree of ethoxylation in the anionic ethoxylated surfactant can range from about 1 to about 3. In another aspect, the average degree of ethoxylation is about 2.

In another exemplary aspect, the hair care compositions comprise: i) at least one crosslinked, nonionic, amphiphilic emulsion polymer; ii) at least one anionic non-ethoxylated surfactant; iii) at least one anionic ethoxylated surfactant; iv) at least one amphoteric surfactant; v) an optional nonionic surfactant; vi) zinc pyrithione antidandruff agent; vi) basic zinc carbonate; and vii) water. In one aspect, the average degree of ethoxylation in the anionic ethoxylated surfactant can range from about 1 to about 3. In another aspect, the average degree of ethoxylation is about 2.

Any amount of the nonionic, amphiphilic emulsion polymeric material can be utilized so long as the amount is sufficient to suspend an insoluble material (e.g., antidandruff agent, silicone, etc.) when included in an aqueous hair care composition comprising at least one surfactant selected from anionic surfactants, amphoteric surfactants, nonionic surfactants, and combinations thereof.

In one aspect, the amount of the polymer that can be incorporated into the aqueous surfactant containing hair care compositions of the disclosed technology ranges from about 0.5 to about 5 wt. % polymer solids (100% active polymer) based on the weight of the total composition. In another aspect, the amount of polymer utilized in the formulation ranges from about 0.75 wt. % to about 3.5 wt. %. In still another aspect, the amount of amphiphilic emulsion polymer employed in the hair care composition ranges from about 1 to about 3 wt. %. In a further aspect, the amount of polymer employed in the hair care composition ranges from about 1.5 wt. % to about 2.75 wt. %. In a still further aspect, the amount of polymer utilized in the hair care composition ranges from about 2 to about 2.5 wt. %, all weights based on the weight of the total composition.

The hair care compositions of the disclosed technology may be in the form of a shampoo, two-in-one shampoo, conditioner, creme rinse, body wash, shower gel, and the like.

In one embodiment, the hair care composition of the disclosed technology is a moderately viscous mixture, having a Brookfield viscosity in the range of from about 1000 mPa·s to about 15,000 mPa·s in one aspect, from about 2,000 mPa·s to about 10,000 mPa·s in another aspect, from about 3,500 mPa·s to about 8,500 mPa·s in still another aspect, and from about 4,500 mPa·s to about 5500 mPa·s in a further aspect. The viscosities are adjustable by changing the amount of nonionic, amphiphilic emulsion polymeric material in the hair care composition. The product should be pourable from a relatively narrow mouth bottle (approximately 1.5 cm in diameter) and the product will not be so thin to run off of the hands or the hair.

Hair care compositions of the present technology are stable indefinitely at temperatures normally found in commercial product storage and shipping. The compositions resist phase separation or settling of composition ingredients at a temperature of about 20° C. to about 25° C. essentially indefinitely. The compositions also must demonstrate sufficient stability to phase separation and settling of ingredients at temperatures normally found in commercial product storage and shipping to remain unaffected for periods of one year or more.

Hair care cleansing compositions employing the nonionic, amphiphilic emulsion polymers of the disclosed technology not only provide compositions in which they are contained with enhanced suspension stability, they also provide other unexpected desirable properties such as foam quality, irritation mitigation, and enhanced silicone deposition.

The hair care compositions of the disclosed technology may be prepared by any known technique. The formulation of hair care antidandruff cleansing compositions are well-known in the formulation art and include conventional formulation and mixing techniques. In one embodiment, the nonionic, amphiphilic emulsion polymers of the disclosed technology can be added to any commercially available antidandruff hair care composition to enhance the suspension stability thereof. Given the pH independent nature of the nonionic, amphiphilic emulsion polymer disclosed herein, it can be added at any point during the commercial production process of antidandruff hair care cleansing products.

The compositions of the present technology can be used in direct application to the hair, scalp and skin in a conventional manner for cleansing skin and hair and controlling dandruff on the skin or scalp. The compositions herein are useful for cleansing the hair and scalp, and other areas of the body such as underarm, feet, and groin areas and for any other area of skin in need of treatment. The present technology may be used for treating or cleansing of the skin or hair of animals as well. An effective amount of the composition for application, typically ranges from about 1 g to about 50 g in one aspect, and from about 1 g to about 20 g in another aspect, for cleansing hair, skin or other area of the body. The composition is topically applied to the hair, skin or other area that has preferably been wetted, generally with water, and then rinsed off. Application to the hair typically includes working the cleansing composition through the hair with the fingers to build up lather.

In one embodiment, one method for providing antidandruff efficacy with a shampoo embodiment comprises the steps of: (a) wetting the hair with water, (b) applying an effective amount of the antidandruff shampoo composition to the hair, and (c) rinsing the antidandruff shampoo composition from the hair using water. These steps may be repeated as many times as desired to achieve the cleansing, conditioning, and anti-dandruff benefits sought.

This technology is illustrated by the following examples that are merely for the purpose of illustration and are not to be regarded as limiting the scope of the technology or the manner in which it can be practiced. Unless specifically indicated otherwise, parts and percentages are given by weight.

### Test Methods

### Yield Stress

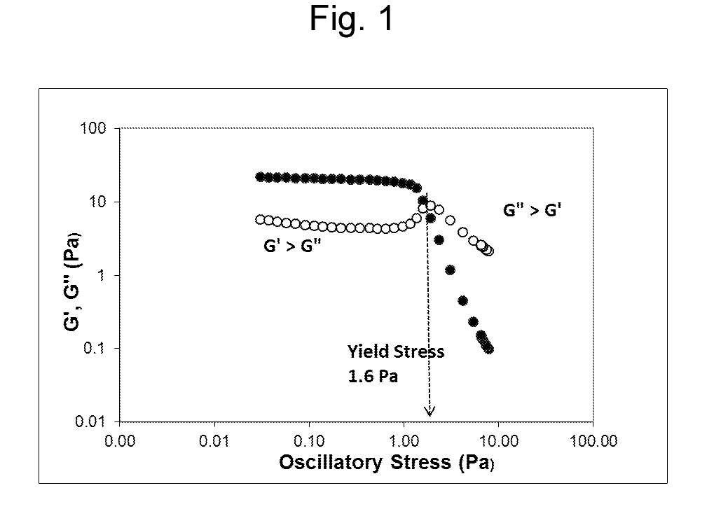

The yield stress values of these polymers are determined by oscillatory and steady shear measurements on a controlled stress rheometer (TA Instruments AR1000N rheometer, New Castle, Del.) utilizing parallel plate geometry (40 mm stainless steel plate with a 1000 μm gap) at 25° C. The oscillatory measurements are performed at a fixed frequency of 1 rad/sec. The elastic and viscous moduli (G′ and G″ respectively) are obtained as a function of increasing stress amplitude. In cases where the swollen polymer particles create a network, G′ is larger than G″ at low stress amplitudes but decreases at higher amplitudes crossing G″ because of rupture of the network. As illustrated in FIG. 1 the stress corresponding to the crossover of G′ and G″ is noted as the yield stress.

### Viscosity (Brookfield)

Brookfield rotating spindle method (all viscosity measurements reported herein are conducted by the Brookfield method whether mentioned or not): The viscosity measurements are calculated in mPa·s, employing a Brookfield rotating spindle viscometer, Model RVT (Brookfield Engineering Laboratories, Inc.), at about 20 revolutions per minute (rpm), at ambient room temperature of about 20 to 25° C. (hereafter referred to as viscosity). Spindle sizes are selected in accordance with the standard operating recommendations from the manufacturer. Generally, spindle sizes are selected as follows:

| Spindle Size No. | Viscosity Range (mPa · s) |
|---|---|
| 1 | 1-50 |
| 2 | 500-1,000 |
| 3 | 1,000-5,000 |
| 4 | 5,000-10,000 |
| 5 | 10,000-20,000 |
| 6 | 20,000-50,000 |
| 7 | >50,000 |

The spindle size recommendations are for illustrative purposes only. The artisan of ordinary skill in the art will select a spindle size appropriate for the system to be measured.

### Stability

The various hair care products or compositions made using the nonionic, amphiphilic emulsion polymers rheology of the present technology are stable. The stability requirements for a particular composition will vary with its end marketplace application as well as the geography in which it is to be bought and sold. An acceptable “shelf life” is subsequently determined for each composition. This refers to the amount of time that a composition should be stable across its normal storage and handling conditions, measured between the times that the composition is produced and when it is finally sold for consumer use. Generally, Personal Care compositions require a 1 to 3 year shelf life.

To eliminate the need to conduct stability studies in excess of one year, the formulator will conduct stability testing at stressed conditions in order to predict the shelf life of a composition. Typically, accelerated testing is conducted at elevated static temperatures, usually 45-50° C. A composition should be stable for at least 2 weeks, desirably 1 month, preferably 2 or 3 months, and most preferably 4 or 5 months at 45° C. Additionally, freeze-thaw cycling is often employed wherein the composition is cycled between a freezing temperature, usually 0° C., and an ambient temperature, usually 20-25° C. A composition should pass a minimum of 1 freeze-thaw cycle, preferably 3 cycles, and most preferably 5 cycles.

Products or compositions made according to the present technology are considered stable if they meet one or more of the following criteria:

1. There is no phase separation, settling, or creaming of any material in the composition. The composition should remain completely homogenous throughout its bulk. Separation is herein defined as the visible existence of 2 or more distinct layers or phases of any component in the formulation, including but not limited to insoluble matter, soluble matter, oily substances and the like.
2. The viscosity of the composition does not significantly increase or decrease over time, generally less than 50%, preferably less than 35%, and most preferably less than 20%.
3. The pH of the composition does not increase or decrease more than two pH units, preferably not more than one unit, and most preferably not more than one-half unit.
4. The rheology and texture of the composition does not significantly change over time to that which is unacceptable.

Products or compositions made according to the present technology are considered unstable if they do not meet one or more of the above listed criteria. Further information on stability testing requirements can be found in “The Fundamentals of Stability Testing; IFSCC Monograph Number 2”, published on behalf of the International Federation of Societies of Cosmetic Chemists by Micelle Press, Weymouth, Dorset, England, and Cranford, N.J., U.S.A. and is herein incorporated by reference.

### Hair Tress Preparation Procedure for Silicone Deposition Testing

Tresses of commercially blended untreated (virgin) human hair are prepared employing natural brown or black color European hair supplied by International Hair Importers and Products Inc., New York. The tresses used for this test are comprised of European brown hair, weighing 0.5 g, 7 inches long and 0.5 inches wide with a sewn/glued flat binding. Prior to treatment, each tress is washed with a dilute aqueous solution of sodium lauryl sulfate (10% SLS) followed by thorough rinsing with de-ionized water at ambient room temperature. The tresses are dried by towel blotting.

The damp tress is placed on top of a weighing dish and 0.25 g of the test shampoo formulation is applied evenly down the length of the tress. The shampoo is massaged into the swatch and the tress is then rinsed under warm tap water for approximately 60 seconds. The treatment step is repeated a second time for a total of two wash/rinse cycles.

### Silicone Deposition Measurement

The amount of silicone (silicon atoms) deposited on the hair tress samples treated with a test shampoo composition containing the nonionic, amphiphilic emulsion polymer is measured by X-Ray fluorescence (XRF) spectroscopy. A wavelength dispersive XRF spectrometer (PANalytical Axios Advanced Sequential 4 kW spectrometer—Model Number PW4400) interfaced with SuperQ 4 software application and fitted with a rhodium tube with an InSb crystal is utilized to facilitate high sensitivity for silicon atom detection corresponding to Si K alpha band. The samples are analyzed using a qualitative program to measure intensities across a two-theta scan range from 139.75° to 147.99° with a peak maximum at 144.53°. The samples are scanned in a vacuum environment using a tube voltage of 25 kV and a current of 160 mA. Scanning speed is 0.05° 2-Theta/sec. with 0.02° 2-Theta step size.

X-rays from the instrument excite silicon atoms deposited on the surface of the wool swatch causing them to emit energy and fluoresce. The silicon fluorescence is detected and recorded as counts per second (kcps). Higher count rates are indicative of higher silicon atom deposition. The amount of silicon atoms detected is directly proportional to the amount of silicone conditioner deposited on the hair. Samples for XRF analysis are prepared by folding each of the treated wool swatches and placing the folded swatch into a sample cup having a 6μ thick polyethylene support substrate formed into the bottom. A polyethylene spacer is placed on each swatch to hold it onto the substrate. An average reading of 3 hair tresses per formulation is reported. Results are reported as average Peak Si Intensity (kcps). Higher kcps values indicate higher levels of silicon atom deposition.

The following abbreviations and trade names are utilized in the examples.

### Abbreviations and Trade Names

| | |
|---|---|
| AMD | Acrylamide |
| AMPS ® Monomer | 2-Acrylamido-2-Methylpropanesulfonic Acid, |
| | Lubrizol Advanced Materials, Inc. |
| AN | Acrylonitrile |
| APE | Allyl Pentaerythritol |
| n-BA | n-Butyl Acrylate |
| BDGMA | Butyl Diglycol Methacrylate |
| BEM | Sipomer ® Ethoxylated (25) Behenyl |
| | Methacrylate, Rhodia |
| i-BMA | iso-Butyl Methacrylate |
| s-BMA | sec-Butyl Methacrylate |
| ChembetaineTM CAD | Cocamidopropyl Betaine (amphoteric |
| | surfactant), Lubrizol Advanced Materials, Inc. |
| | (35% active) |
| CSEM | Visiomer ® C18 PEG 1105 MA W |
| | Polyethyleneglycol (25) Cetearyl Methacrylate, |
| | Evonik Röhm GmbH |
| CYCLO | Cyclohexane |
| Celvol ® 502 PVA | Polyvinyl Alcohol (hydrolysis % = 87-89%), |
| | Celanese Corpoartion |
| EA | Ethyl Acrylate |
| EMA | Ethyl Methacrylate |
| E-Sperse RS-1596 | Reactive surfactant with one polymerizable |
| | reactive group from Ethox Chemical, LLC |
| E-Sperse RS-1616 | Reactive surfactant with one polymerizable |
| | reactive group from Ethox Chemical, LLC |
| E-Sperse RS-1617 | Amphiphilic crosslinker with two polymerizable |
| | reactive groups from Ethox Chemical, LLC |
| E-Sperse RS-1618 | Amphiphilic crosslinker with two polymerizable |
| | reactive groups from Ethox Chemical, LLC |
| E-Sperse RS-1684 | Amphiphilic crosslinker with two polymerizable |
| | reactive groups from Ethox Chemical, LLC |
| 2-HEA | 2-Hydroxyethyl Acrylate |
| HEMA | 2-Hydroxyethyl Methacrylate |
| HPA | Hydroxypropyl Acrylate |
| HPMA | 3-Hydroxypropyl Methacrylate |
| LEM | Blemmer ® PLE-200 Lauroxy |
| | Polyethyleneglycol Methacrylate, NOF |
| | Corporation |
| LMA | Lauryl Methacrylate |
| MA | Methyl Acrylate |
| MAA | Methacrylic Acid |
| MA EO/PO-300 | Blemmer ® 50PEP-300 Polyethyleneglycol |
| | (3.5) Polypropyleneglycol (2.5) Methacrylate, |
| | NOF Corporation |
| MA EO/PO-800 | , Blemmer ® 55PET-800 Polyethyleneglycol |
| | (10) Polypropyleneglycol (5) Methacrylate, |
| | NOF Corporation |
| MAMD | Methacrylamide |
| MMA | Methyl Methacrylate |
| MPEG 350 | Bisomer ® 350 MA Methoxy Polyethyleneglycol |
| | (8) Methacrylate, GEO Specialty Chemicals |
| MPEG 400 | Blemmer ® PME-400 Methoxy |
| | Polyethyleneglycol (9) Methacrylate, NOF |
| | Corporation |
| MPEG S10 W | Bisomer ® S10 W Methoxy Polyethyleneglycol |
| | (23) Methacrylate, GEO Specialty Chemicals |
| NPEA-1300 | Blemmer ® ANE-1300, Nonylphenoxy |
| | Polyethyleneglycol (30) Acrylate, NOF |
| | Corporation |
| OEO/POMA | Blemmer ® 50POEP-800B Octoxy |
| | Polyethyleneglycol (8) Polypropyleneglycol (6) |
| | Methacrylate, NOF Corporation (hydrophobe = |
| | 2-ethylhexyl) |
| PEA | Blemmer ® AAE-300 Phenoxy |
| | Polyethyleneglycol (5.5) acrylate, NOF |
| | Corporation |
| PEO/POMA | Blemmer ® 43PAPE-600B Phenoxy |
| | Polyethyleneglycol (6) Polypropyleneglycol (6) |
| | Methacrylate, NOF Corporation |
| SEM-400 | Blemmer ® PSE-400 Stearoxy |
| | Polyethyleneglycol (9) Methacrylate, NOF |
| | Corporation |
| SEM-1300 | Blemmer ® PSE-1300 Stearoxy |
| | Polyethyleneglycol (30) Methacrylate, NOF |
| | Corporation |
| SMA | Stearyl Methacrylate |
| Selvol ® 502 and | Polyvinyl Alcohol (hydrolysis % = 87-89%), |
| 205 PVA | Sekisui Corporation |
| STYSEM-25 | Sipomer ®, ω-Tristyrylphenyl Polyoxyethylene |
| | (25) Methacrylate) |
| Sulfochem ™ ALS-K | Ammonium Lauryl Sulfate (anionic surfactant |
| | preserved with Kathon ® CG preservative from |
| | Rohm and Haas Company), Lubrizol Advanced |
| | Materials, Inc. (30% active) |
| SulfochemTM ES-2 | Sodium Laureth Sulfate - 2 moles of |
| | ethoxylation (anionic surfactant), Lubrizol |
| | Advanced Materials, Inc. (26% active) |
| Sulfochem ™ SLS | Sodium Lauryl Sulfate (anionic surfactant), |
| | Lubrizol Advanced Materials, Inc. (30% active) |
| Sulfochem ™ TLS | TEA-Lauryl Sulfate (anionic surfactant) Lubrizol |
| | Advanced Materials, Inc. (40% active) |
| TBHP | tert-butyl t-butyl hydroperoxide |
| VA | Vinyl Acetate |
| VA-10 | Vinyl Decanoate |
| VP | N-Vinylpyrrolidone |
| i-PAMD | iso-Propylacrylamide |
| MAMD | Methacrylamide |

### Example 1

### Monomer Composition=EA/n-BA/HEMA/BEM (35/15/45/5) Wt.

An emulsion polymer was prepared as follows. A monomer premix was made by mixing 140 grams of DI water, 5 grams of E-Sperse® RS-1618 amphiphilic crosslinker, 175 grams of (EA), 75 grams of (n-BA), 225 grams of (HEMA) and 33.3 grams of (BEM). Initiator A was made by mixing 2.86 grams of TBHP in 40 grams of DI water. Reductant A was prepared by dissolving 0.13 grams of erythorbic acid in 5 grams of DI water. Reductant B was prepared by dissolving 2.0 grams of erythorbic acid in 100 grams of DI water. A 3-liter reactor was charged with 800 grams of DI water, 10 grams of 40% AOS and 25 grams of Selvol® 502 PVA. The contents of the reactor were heated to 70° C. under a nitrogen blanket with agitation. After holding the reactor contents at 70° C. for one hour, initiator A was added to the reactor followed by addition of reductant A. After about 1 minute, the monomer premix was metered into the reaction vessel over a period of 180 minutes. About 3 minutes after the start of monomer premix introduction, reductant B was metered to the reactor over a period of 210 minutes. The reaction temperature was kept at 65° C. After completion of reductant B feed, the temperature of the reaction vessel contents was maintained at 65° C. for 60 minutes. The reactor contents were then cooled to 60° C. A solution of 1.79 grams of TBHP and 0.13 grams of 40% AOS in 15 grams of DI water was added to the reactor. After 5 minutes, a solution of 1.05 grams of erythorbic acid in 15 grams of DI water was added to the reactor. The reactor contents were maintained at 60° C. After 30 minutes, a solution of 1.79 grams of TBHP and 0.13 grams of 40% AOS in 15 grams of DI water was added to the reactor. After 5 minutes, a solution of 1.05 grams of erythorbic acid in 15 grams of DI water was added to the reactor. The reactor contents were maintained at 60° C. for about 30 minutes. Then, the reactor was cooled to room temperature and filtered through 100 micron filter cloth. The pH of the resulting emulsion was adjusted to 4.5 with ammonium hydroxide. The polymer product had a solids content of 30.4%, a viscosity of 21 cps, and a particle size of 119 nm.

### Example 2

### Monomer Composition=EA/n-BA/HEMA/BEM (35/15/45/5) Wt.

An emulsion polymer was prepared as follows. A monomer premix was made by mixing 140 grams of DI water, 5 grams of E-Sperse® RS-1618 amphiphilic crosslinker, 175 grams of (EA), 75 grams of (n-BA), 225 grams of (HEMA) and 33.3 grams of (BEM). Initiator A was made by mixing 3.57 grams of TBHP in 40 grams of DI water. Reductant A was prepared by dissolving 0.13 grams of erythorbic acid in 5 grams of DI water. Reductant B was prepared by dissolving 2.5 grams of erythorbic acid in 100 grams of DI water. A 3-liter reactor was charged with 825 grams of DI water, 7.5 grams of 40% AOS and 15 grams of Selvol® 502 PVA, and then the contents were heated to 70° C. under a nitrogen blanket with agitation. After holding the reactor contents at 70° C. for one hour, the contents of the reactor were cooled down to 65° C., and then initiator A was then added to the reactor followed by addition of reductant A. After about 1 minute, the monomer premix was metered into the reaction vessel over a period of 180 minutes. About 3 minutes after the start of monomer premix introduction, reductant B was metered into the reactor over a period of 210 minutes. After completion of the reductant B feed, the temperature of the reaction vessel contents was maintained at 65° C. for 60 minutes. The reactor contents were then cooled to 60° C. A solution of 1.96 grams of TBHP and 0.13 grams of 40% AOS in 15 grams of DI water was added to the reactor. After 5 minutes, a solution of 1.27 grams of erythorbic acid in 15 grams of DI water was added to the reactor. The reactor contents were maintained at 60° C. After 30 minutes, a solution of 1.96 grams of TBHP and 0.13 grams of 40% AOS in 15 grams of DI water was added to the reactor. After 5 minutes, a solution of 1.27 grams of erythorbic acid in 15 grams of DI water was added to the reactor. The reactor contents were maintained at 60° C. for about 30 minutes. Then, the reactor contents were cooled to room temperature (22° C.) and filtered through 100 micron filter cloth. The pH of the resulting emulsion was adjusted to 4.5 with ammonium hydroxide. The polymer product had a solids content of 30.85%, a viscosity of 19 cps, and a particle size of 99 nm.

### Example 3

### Monomer Composition=EA/n-BA/HEMA/BEM/APE (35/15/45/5) Wt.

An emulsion polymer employing APE crosslinker was prepared as follows. A monomer premix was made by mixing 140 grams of DI water, 5 grams of E-Sperse® RS-1618 amphiphilic crosslinker, 175 grams of (EA), 70.6 grams of (n-BA), 225 grams of (HEMA) and 33.3 grams of (BEM). Initiator A was made by mixing 3.57 grams of TBHP in 40 grams of DI water. Reductant A was prepared by dissolving 0.13 grams of erythorbic acid in 5 grams of DI water. Reductant B was prepared by dissolving 2.5 grams of erythorbic acid in 100 grams of DI water. A 3-liter reactor was charged with 800 grams of DI water, 10 grams of 40% AOS and 25 grams of Selvol® 502 PVA and then was heated to 70° C. under a nitrogen blanket with proper agitation. After holding the reactor at 70° C. for one hour, initiator A was added to the reactor and followed by addition of reductant A. After about 1 minute, the monomer premix was metered to the reaction vessel over a period of 180 minutes. About 3 minutes after the start of monomer premix introduction, reductant B was metered to the reactor over a period of 210 minutes. The reaction temperature was kept at 65° C. At about 115 minutes after the monomer premix introduction, the premix metering was stopped for 10 minutes, and then 0.44 grams of 70% APE in 3.94 grams of n-BA was added to the monomer premix. After the 10 minute period, the premix metering was resumed. After completion of reductant B feed, the temperature of the reaction vessel contents was maintained at 65° C. for 60 minutes. The reactor contents were then cooled to 60° C. A solution of 1.96 grams of TBHP and 0.13 grams of 40% AOS in 15 grams of DI water was added to the reactor. After 5 minutes, a solution of 1.27 grams of erythorbic acid in 15 grams of DI water was added to the reactor. The reactor contents were maintained at 60° C. After 30 minutes, a solution of 1.96 grams of TBHP and 0.13 grams of 40% AOS in 15 grams of DI water was added to the reactor. After 5 minutes, a solution of 1.27 grams of erythorbic acid in 15 grams of DI water was added to the reactor. The reactor contents were maintained at 60° C. for about 30 minutes. Then, the reactor contents were cooled to room temperature and filtered through 100 micron filter cloth. The pH of the resulting emulsion was adjusted to 4.5 with ammonium hydroxide. The polymer product had a solids content of 30.8%, a viscosity of 24 cps and a particle size of 110 nm.

### Example 4

### Monomer Composition=EA/n-BA/HEMA/BEM (35/15/45/5) Wt.

An emulsion polymer was prepared as follows. A monomer premix was made by mixing 140 grams of DI water, 5 grams of 100% E-Sperse® RS-1617 amphiphilic crosslinker, 175 grams of (EA), 75 grams of (n-BA), 225 grams of (HEMA) and 33.3 grams of (BEM). Initiator A was made by dissolving 5 grams of Azo VA-086 in 40 grams of DI water. Initiator B was prepared by dissolving 2.5 grams of Azo VA-086 in 100 grams of DI water. A 3-liter reactor was charged with 800 grams of DI water, 5 grams of 40% AOS and 10 grams of Selvol® 203 PVA. The contents of the reactor were heated to 87° C. under a nitrogen blanket with agitation. After holding the reactor contents at 87° C. for one hour, initiator A was added to the reactor. After about 1 minute, the monomer premix was metered to the reaction vessel over a period of 120 minutes. About 3 minutes after the start of monomer premix introduction, initiator B was metered to the reactor over a period of 150 minutes. The reaction temperature was maintained at 87° C. After completion of the initiator B feed, the temperature of the reaction vessel contents were maintained at 87° C. for 60 minutes. The reactor contents were then cooled to 49° C. A solution of 0.61 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. After 30 minutes, a solution of 0.69 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. for about 30 minutes. The reactor was cooled to the room temperature and filtered through 100 micron filter cloth. The pH of the resulting emulsion was adjusted to 4.5 with ammonium hydroxide. The polymer product had a solids content of 31.4%, a viscosity of 14 cps and a particle size of 105 nm.

### Example 5

### Monomer Composition=EA/n-BA/HEMA/BEM (30/20/45/5) Wt.

An emulsion polymer was prepared in the same manner as Example 4 except that the monomer amount was changed to 30 wt. % (EA), 20 wt. % (n-BA), 45 wt. % (HEMA) and 5 wt. % (BEM) instead of 35 wt. % (EA), 15 wt. % (n-BA), 45 wt. % (HEMA) and 5 wt. % (BEM). The polymer product had a solids content of 30.8%, a viscosity of 26 cps and a particle size of 83 nm.

### Example 6

### Monomer Composition=EA/n-BA/HEMA/BEM (25/25/45/5) Wt.

An emulsion polymer was prepared in the same manner as in Example 4 except that the monomer amount was changed to 25 wt. % (EA), 25 wt. % (n-BA), 45 wt. % (HEMA) and 5 wt. % (BEM) instead of 35 wt. % (EA), 15 wt. % (n-BA), 45 wt. % (HEMA) and 5 wt. % (BEM). The polymer product had a solids content of 30.9%, a viscosity of 39 cps and a particle size of 78 nm.

### Example 7

### Monomer Composition=EA/n-BA/HEMA/BEM (35/20/40/5) Wt.

An emulsion polymer was prepared in the same manner as Example 4 except that the monomer compositions were changed to 35 wt. % (EA), 20 wt. % (n-BA), 40 wt. % (HEMA) and 5 wt. % (BEM) instead of 35 wt. % (EA), 15 wt. % (n-BA), 45 wt. % (HEMA) and 5 wt. % (BEM). The polymer product had a solids content of 31.4%, a viscosity of 42 cps and a particle size of 87 nm.

### Example 8

### Monomer Composition=EA/n-BA/BEM/HEMA/AA (35/15/5/43/2) Wt.

An emulsion polymer was prepared as follows. A monomer premix was made by mixing 70 grams of DI water, 2.5 grams of E-Sperse® RS-1618 amphiphilic crosslinker, 87.5 grams of (EA), 37.5 grams of (n-BA), 16.67 grams of (BEM), 107.5 grams of (HEMA), and 5 grams of (AA). Initiator No. 1 was made by dispersing 2.5 grams of VA-086 in 20 grams of DI water. Initiator No. 2 was prepared by dissolving 1.25 grams of VA-086 in 50 grams of DI water. A 1-liter reactor vessel was charged with 400 grams of DI water, 2.5 grams of 40% AOS and 5 grams of Selvol® 203 PVA and then the contents were heated to 87° C. under a nitrogen blanket and agitation. Initiator No. 1 was added to the reaction vessel. The monomer premix was then metered into the reaction vessel over a period of 120 minutes; while at the same time, initiator No. 2 was metered to the reaction vessel over a period of 150 minutes. After the completion of monomer premix feed, 16.5 grams of DI water was added to the dropping funnel which held the monomer premix to flush out the residual monomers into the reaction mixture. After the completion of initiator No. 2 feed, the temperature of the reaction vessel contents was maintained at 87° C. for 60 minutes. The reaction vessel contents were then cooled to 49° C. A solution of 0.3 grams of TBHP and 0.14 grams of 40% AOS in 7.5 grams of DI water was added to the reaction vessel. After 5 minutes, a solution of 0.3 grams of erythorbic acid in 7.5 grams of DI water was added to the reaction vessel. After 30 minutes, another solution of 0.3 grams of TBHP and 0.14 grams of 40% AOS in 7.5 grams of DI water was added to the reaction vessel. A solution of 0.3 grams of erythorbic acid in 7.5 grams of DI water was then added to the reaction vessel after 5 minutes. The reaction vessel contents were maintained at 60° C. for another 30 minutes. The reaction vessel contents were then cooled to room temperature (23° C.) and filtered through a 100 micron filter cloth. The pH of the resulting emulsion was adjusted to 3.5 to 4.5 with 28% ammonium hydroxide in DI water. The resulting polymer latex had a solids level of 30.7%, and a particle size of 113 nm.

### Example 9

### Monomer Composition=EA/n-BA/BEM/HEMA/AMD (35/15/5/43/2) Wt.

An emulsion polymer was prepared as follows. A monomer premix was made by mixing 70 grams of DI water, 2.5 grams of E-Sperse® RS-1618 amphiphilic crosslinker, 87.5 grams of (EA), 37.5 grams of (n-BA), 16.67 grams of (BEM), 107.5 grams of 2-hydroxyl ethyl methacrylate (HEMA), and 10 grams 50% acrylamide (AMD). Initiator No. 1 was made by dispersing 2.5 grams of VA-086 in 20 grams of DI water. Initiator No. 2 was prepared by dissolving 1.25 grams of VA-086 in 50 grams of DI water. A 1-liter reactor vessel was charged with 400 grams of DI water, 2.5 grams of 40% AOS and 5 grams of Selvol® 203 PVA. The contents of the vessel was heated to 87° C. under a nitrogen blanket and agitation. Initiator No. 1 was added to the reaction vessel. The monomer premix was then metered to the reaction vessel over a period of 120 minutes; while at the same time, initiator No. 2 was metered to the reaction vessel over a period of 150 minutes. After the completion of monomer premix feed, 16.5 grams of DI water was added to the dropping funnel which held the monomer premix to flush out the residual monomers. After the completion of initiator No. 2 feed, the temperature of the reaction vessel contents was maintained at 87° C. for 60 minutes. The reaction vessel contents were then cooled to 49° C. A solution of 0.3 grams of TBHP and 0.14 grams of 40% AOS in 7.5 grams of DI water was added to the reaction vessel. After 5 minutes, a solution of 0.3 grams of erythorbic acid in 7.5 grams of DI water was added to the reaction vessel. After 30 minutes, another solution of 0.3 grams of TBHP and 0.14 grams of 40% AOS in 7.5 grams of DI water was added to the reaction vessel. A solution of 0.3 grams of erythorbic acid in 7.5 grams of DI water was then added to the reaction vessel after 5 minutes. The reaction vessel contents were maintained at 60° C. for another 30 minutes. Then, the reaction vessel contents were cooled to room temperature and filtered through a 100 micron filter cloth. The pH of the resulting emulsion was adjusted to 3.5-4.5 with 28% ammonium hydroxide solution. The resulting polymer latex had a solid level of 30.4%, and a particle size of 90.4 nm.

### Example 10

### Monomer Composition=EA/n-BA/BEM/HEMA/MAMD (35/15/5/43/2) Wt.

An emulsion polymer was prepared as follows. A monomer premix was made by mixing 70 grams of DI water, 2.5 grams of E-Sperse® RS-1618 amphiphilic crosslinker, 87.5 grams of (EA), 37.5 grams of (n-BA), 16.67 grams of (BEM), 107.5 grams of (HEMA), and 20 grams 25% (MAMD). Initiator No. 1 was made by dispersing 2.5 grams of VA-086 in 20 grams of DI water. Initiator No. 2 was prepared by dissolving 1.25 grams of VA-086 in 50 grams of DI water. A 1-liter reactor vessel was charged with 400 grams of DI water, 2.5 grams of 40% AOS and 5 grams of Selvol® 203 PVA, and then the contents heated to 87° C. under a nitrogen blanket and agitation. Initiator No. 1 was added to the reaction vessel. The monomer premix was then metered to the reaction vessel over a period of 120 minutes; while at the same time, initiator No. 2 was metered to the reaction vessel over a period of 150 minutes. After the completion of monomer premix feed, 16.5 grams of DI water was added to the dropping funnel which held the monomer premix to flush out the residual monomers. After the completion of initiator No. 2 feed, the temperature of the reaction vessel was maintained at 87° C. for 60 minutes. The reaction vessel was then cooled to 49° C. A solution of 0.3 grams of TBHP and 0.14 grams of 40% AOS in 7.5 grams of DI water was added to the reaction vessel. After 5 minutes, a solution of 0.3 grams of erythorbic acid in 7.5 grams of DI water was added to the reaction vessel. After 30 minutes, another solution of 0.3 grams of TBHP and 0.14 grams of 40% AOS in 7.5 grams of DI water was added to the reaction vessel. A solution of 0.3 grams of erythorbic acid in 7.5 grams of DI water was added to the reaction vessel after 5 minutes. The reaction vessel contents were maintained at 60° C. for another 30 minutes. The reaction vessel contents were cooled to room temperature and filtered through a 100 micron filter cloth. The pH of the resulting emulsion was adjusted to 3.5-4.5 with a 28% ammonium hydroxide solution. The resulting polymer latex had a solid level of 26.2%, and a particle size of 100 nm.

### Example 11

### Monomer Composition=EA/n-BA/BEM/HEMA/BEM (20.5/27.5/45/7) Wt.

An emulsion polymer was prepared as follows. A monomer premix was made by mixing 140 grams of DI water, 5 grams of E-Sperse® RS-1618 amphiphilic crosslinker, 102.5 grams of (EA), 137.5 grams of (n-BA), 175 grams of (HEMA), 46.67 grams of (BEM). Initiator A was made by dissolving 5 grams of Azo VA-086 in 40 grams of DI water. Initiator B was prepared by dissolving 2.5 grams of Azo VA-086 in 100 grams of DI water. A 3-liter reactor was charged with 800 grams of DI water, 5 grams of 40% sodium alpha olefin sulfonate (AOS) and 10 grams of Selvol® 203 PVA, and then the contents were heated to 87° C. under a nitrogen blanket with agitation. After holding the reactor contents at 87° C. for one hour, initiator A was then added to the reactor. After about 2 to 3 minutes, the monomer premix was metered into the reaction vessel over a period of 120 minutes. About 1 minute after the start of monomer premix metering, Initiator B was metered into the reactor over a period of 150 minutes. The reaction temperature was kept at 87° C. After completion of initiator B feed, the temperature of the reaction vessel contents were maintained at 87° C. for 60 minutes. The reactor contents were then cooled to 49° C. A solution of 0.61 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. After 30 minutes, a solution of 0.69 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. for about 30 minutes. The contents of the reactor were cooled to room temperature and filtered through 100 micron filter cloth. The pH of the resulting emulsion was adjusted to 4 to 5 with a 28% ammonium hydroxide solution. The polymer was diluted with 340 grams of DI water to attain a solids 25.1%, a viscosity 13 cps, and particle size 82 nm.

### Example 12

### Monomer Composition=n-VP/EA/BA/VAc/HEMA (20/15/20/20/25) Wt.

An emulsion polymer was prepared as follows. A monomer premix was made by mixing 70 grams of DI water, 2.5 grams of E-Sperse® RS-1618 amphiphilic crosslinker, 50 grams of (n-VP), 37.5 grams of (EA), 50 grams of (n-BA), 50 grams of vinyl acetate (VAc), and 62.5 grams of (HEMA). Initiator 1 was made by mixing 1.07 grams of TBHP in 20 grams of DI water. Reductant 2 was prepared by dissolving 0.83 grams of erythorbic acid in 50 grams of DI water. A 1 liter reactor vessel was charged with 400 grams of DI water, 2.5 grams of 40% AOS and 12.5 grams of Selvol® 502 PVA, and then the contents were heated to 65° C. under a nitrogen blanket and agitation. Initiator 1 was added to the reaction vessel. After about 1 minute, the monomer premix was metered into the reaction vessel over a period of 120 minutes; while at the same time Reductant 2 was metered to the reaction vessel for over a period of 150 minutes. After the completion of monomer premix feed, 16.5 grams of DI water was added to flush the residual monomers from the premix vessel into the reaction vessel. After the completion of Reductant 2 feed, the temperature of the reaction vessel contents were maintained at 65° C. for 60 minutes. The reaction vessel contents were then cooled to 50° C. A solution of 0.3 grams of TBHP and 7.5 grams of DI water was added to the reaction vessel. After 5 minutes, a solution of 0.29 grams of erythorbic acid in 7.5 grams of DI water was added to the reaction vessel. After 30 minutes, a solution of 0.32 grams of TBHP and 7.5 grams of DI water was added to the reaction vessel. After 5 minutes, a solution of 0.29 grams of erythorbic acid in 7.5 grams of DI water was added to the reaction vessel. The reaction vessel contents were maintained at 50° C. for about 30 minutes. Then, the reaction vessel was cooled to room temperature (22° C.) and filtered through 100 micron filter cloth. The resulting polymer latex had a solids level 30.8%, and particle size 100 nm.

### Example 13

### Monomer Composition=EA/n-BA/HEMA/n-VP/CSEM (23/20/35/20/2) Wt.

An emulsion polymer was prepared as follows. A monomer premix was made by mixing 140 grams of DI water, 5 grams of E-Sperse® RS-1618 amphiphilic crosslinker, 115 grams of (EA), 100 grams of (n-BA), 175 grams of (HEMA), 12.5 grams (CSEM), and 100 grams of (n-VP). Initiator A was made by dissolving 4 grams Azo VA-086 in 40 grams of DI water. Initiator B was prepared by dissolving 0.75 grams of Azo VA-086 in 100 grams of DI water. A 3-liter reactor was charged with 800 grams of DI water, 5 grams of 40% AOS and 20 grams of Selvol® 203 PVA, and then the contents were heated to 87° C. under a nitrogen blanket with mild agitation. After holding the reactor contents at 87° C. for one hour Initiator A was then added to the reactor. After about 1 minute, the monomer premix was metered into the reaction vessel for over a period of 120 minutes. About 3 minutes after the start of monomer premix introduction, Initiator B was metered into the reactor over a period of 150 minutes. The reaction temperature was maintained at 87° C. After completion of the Initiator B feed, the temperature of the reaction vessel contents were maintained at 87° C. for an additional 60 minutes. The reactor contents were then cooled to 49° C. A solution of 0.61 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. After 30 minutes, a solution of 0.69 grams of TBHP and 0.29 grams of 40% AOS in 15 grams of DI water was added to the reactor. After 5 minutes, a solution of 0.59 grams of erythorbic acid in 15 grams of DI water was added to the reactor. The reactor contents were maintained at 49° C. for about 30 minutes. The reactor contents were then cooled to the room temperature (23° C.) and filtered through 100 micron filter cloth. The pH of the resulting emulsion was adjusted to 4.5 with 10% ammonium hydroxide in water. The polymer emulsion had a solids content of 30.9%, a Brookfield viscosity of 36 cps, and particle size 113 nm.

### Example 14

An antidandruff shampoo formulation is formulated with the components set forth in the Table below.

TABLE 5

| | | | |
|---|---|---|---|
| | Total Active | Active | Total Weight |
| Ingredient | (wt. %) | (wt. %) | (g) |

Phase 1

| | | | |
|---|---|---|---|
| SLEL-2—Sodium | 27.3 | 12.0 | 219.78 |
| Laureth Sulfate (2 | | | |
| moles ethoxylation) | | | |
| SLS—Sodium Lauryl | 29.00 | 2.00 | 34.48 |
| Sulfate | | | |
| Cocamide MEA | 100.00 | 0.50 | 2.50 |

Phase 2

| | | | |
|---|---|---|---|
| Deionized Water | — | — | 75.00 |
| Polymer | 30.57 | 2.00 | 32.71 |

Phase 3

| | | | |
|---|---|---|---|
| Kathon ® Preservative | 100.00 | 0.05 | 0.25 |
| Dow Corning ® DC-1491 | 60.00 | 2.00 | 16.67 |
| Silicone Microemulsion | | | |

Phase 4

| | | | |
|---|---|---|---|
| D.I. Water | — | — | 50.0 |
| Jaguar ® C13-S Guar | 100.00 | 0.20 | 1.00 |
| Hydroxypropyltrimonium | | | |
| Chloride | | | |
| Cocamidopropylbetaine | 35.00 | 0.15 | 2.00 |

Phase 5

| | | | |
|---|---|---|---|
| Quickearl ™ II Pearlizing | 34.00 | 2.00 | 29.41 |
| agent (Sodium Laureth | | | |
| Sulfate (and) Glycol | | | |
| Stearate) | | | |
| Zinc Ormadine ® FPS | 50.00 | 1.00 | 10.00 |
| (zinc pyrithione) | | | |

Phase 6

| | | | |
|---|---|---|---|
| D.I. Water | — | — | 20.0 |
| Zinc Carbonate | 97.00 | 1.00 | 5.15 |
| NaCl | 100.00 | 1.00 | 5.00 |
| MgSO₄ | 100.00 | 0.50 | 2.50 |

Phase 7

| | | | |
|---|---|---|---|
| FD&C Blue #1 | — | — | 2 Drops |
| NaOH | 18.0 aqueous | — | q.s. to pH 7.8 |
| | (wt./wt.) | | |

Procedure:

1. The Phase 1 ingredients are mixed as follows: SLES-2, SLS and Cocamide MEA are combined with gentle mixing and heated to 65-70° C. until a homogenous solution is obtained.
2. The Phase 2 ingredients are mixed as follows: The nonionic, amphiphilic emulsion polymer is added to deionized water with gentle mixing.
3. Once Phase 1 cools to 40° C., Phase 1 is added to Phase 2 under gentle mixing.
4. The Phase 3 ingredients are combined with the Phase 1/Phase 2 mixture in the order listed in Table 5 under mixing.
5. In a separate vessel the ingredients of Phase 4 are combined and mixed until homogeneous and then added to the combined Phase 1/2/3 mixture and mixed until fully dispersed.
6. The ingredients of Phase 5 are added to the combined Phase 1/2/3/4 mixture in the order listed in Table 5 and mixed.
7. Phase 6 is separately prepared by combining the ingredients into a homogeneous mixture. The Phase 6 mixture is then added to the combined Phase 1/2/3/4/5 mixture and homogeneously mixed.
8. FD&C Blue #1 is added to the combined Phase 1/2/3/4/5/6 mixture and the pH is adjusted with 18% sodium hydroxide to 7.8.
9. The final formulated shampoo product is allowed to equilibrate for 24 hours.

The amount of silicon (silicon atoms) deposited on hair tress samples treated with the shampoo composition can be measured by X-Ray fluorescence (XRF) spectroscopy in accordance with the test methodology set forth above.

1. An antidandruff composition comprising in an aqueous medium:
a) at least one surfactant selected from an anionic, amphoteric, and zwitterionic surfactant;
b) at least one antidandruff agent; and
c) a nonionic, amphiphilic emulsion polymer;
wherein said emulsion polymer is prepared from a polymerizable monomer composition comprising at least one hydrophilic monomer and at least one hydrophobic monomer, wherein said hydrophilic monomer is selected from hydroxy(C₁-C₅)alkyl (meth)acrylates, N-vinyl amides, or mixtures thereof; wherein said hydrophobic monomer is selected from esters of (meth)acrylic acid with alcohols containing 1 to 30 carbon atoms, vinyl esters of aliphatic carboxylic acids containing 1 to 22 carbon atoms, vinyl ethers of alcohols containing 1 to 22 carbon atoms, vinyl aromatic monomers, vinyl halides, vinylidene halides, associative monomers, semi-hydrophobic monomers, or mixtures thereof; and at least one amphiphilic crosslinking agent containing more than one unsaturated moiety

2. A composition of claim 1 wherein said the at least one antidandruff agent is selected from a polyvalent metal salt of pyrithione.

3. A composition of claim 2 wherein said the at least one antidandruff agent is selected from at least one calcium, magnesium, barium, strontium, zinc, cadmium, tin, and zirconium metal salt of pyrithione.

4. A composition of claim 3 wherein said the at least one antidandruff agent is zinc pyrithione.

5. A composition of claim 4 further comprising a zinc containing layered material selected from basic zinc carbonate, zinc carbonate hydroxide, hydrozincite, and combinations thereof.

6. A composition of claim 5 wherein said zinc layered material is hydrozincite or basic zinc carbonate.

7. A composition of claim 6 wherein said zinc layered material is basic zinc carbonate.

8. A composition of any of claim 2 wherein said the at least one metal salt of pyrithione is present in an amount ranging from about 0.01 wt. % to about 5 wt. % in one aspect and from about 0.1 wt. % to about 2 wt. % in another aspect.

9. A composition of claim 5 wherein the weight ratio of said zinc layered material to said the at least one metal salt of pyrithione is 5:100 to 10:1.

10. A composition of claim 1 wherein said the at least one antidandruff agent is selected from salicylic acid, elemental sulfur, selenium dioxide, selenium sulfides, azole compounds, hydroxy pyridone compounds, and combinations thereof.

11. A composition of claim 10 wherein said the at least one antidandruff agent is present in an amount ranging from about 0.01 wt. %.

12. A composition according to claim 1 wherein the amount of said emulsion polymer solids ranges from about 0.5 to about 5 wt. % based on the weight of said composition.

13. A composition according to claim 1 wherein the amount of said surfactant ranges from about 5 wt. % to about 30 wt % (active basis) based on the weight of said composition.

14. A composition according to claim 1 wherein the at least one surfactant is selected from an anionic surfactant and an amphoteric or zwitterionic surfactant.

15. A composition according to claim 14 wherein said anionic surfactant is selected from sodium dodecyl sulfate, ammonium dodecyl sulfate, sodium lauryl sulfate, sodium trideceth sulfate, ammonium lauryl sulfate, sodium laureth sulfate, ammonium laureth sulfate or mixtures thereof.

16. A composition according to claim 14 wherein said amphoteric or zwitterionic surfactant is cocamidopropyl betaine.

17. A composition according to claim 1 wherein the amphiphilic crosslinking agent is a compound of formula (III): 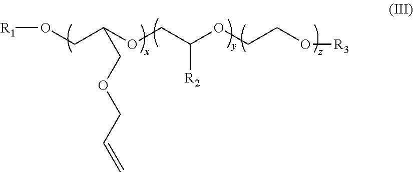
where:
R₁ is a C₁₀₋₂₄ alkyl, alkaryl, alkenyl, or cycloalkyl;
R₂ is CH₃, CH₂CH₃, C₆H₅, or C₁₄H₂₉;
R₃ is H or Z⁻ M⁺
Z⁻ is SO₃⁻, or PO₃²;
M⁺is Na⁺, K⁺, NH₄⁺, or an alkanolamine;
x is 2-10;
y is 0-200; and
z is 4-200.

18. A composition according to claim 1 wherein the amphiphilic crosslinking agent is a compound of formula (IV): 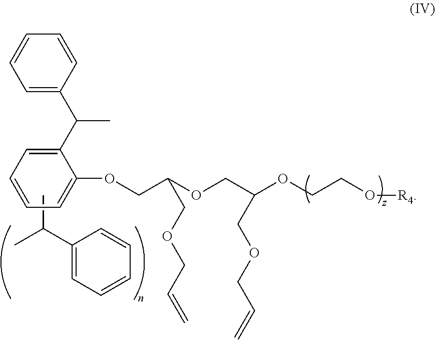
where:
n is 1 or 2;
z is 4 to 40 in one aspect; and
R₄ is H, SO₃⁻M⁺or PO₃⁻M⁺, and M is selected from Na⁺, K⁺, NH₄⁺or an alkanolamine.

19. A composition according to claim 1 wherein said hydroxy(C₁-C₅)alkyl (meth)acrylate is selected from at least one compound represented by the formula: 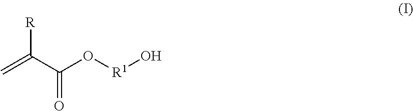
wherein R is hydrogen or methyl and R¹ is an divalent alkylene moiety containing 1 to 5 carbon atoms, wherein the alkylene moiety optionally can be substituted by one or more methyl groups.

20. A composition according to claim 1 wherein said N-vinyl amide is selected from a N-vinyllactam containing 4 to 9 atoms in the lactam ring moiety, wherein the ring carbon atoms, optionally, can be substituted by one or more C₁-C₃ lower alkyl group.

21. A composition according to claim 1 wherein said ester of (meth)acrylic acid with alcohols containing 1 to 30 carbon is selected from at least one compound represented by the formula: 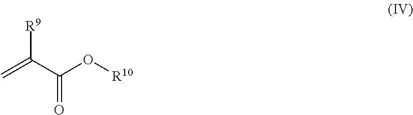
wherein R⁹ is hydrogen or methyl and R¹⁰ is C₁ to C₂₂ alkyl.

22. A composition according to claim 1 wherein said vinyl ester of aliphatic carboxylic acids containing 1 to 22 carbon atoms is selected from at least one compound represented by the formula: 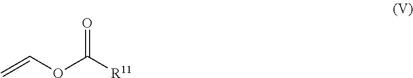
wherein R¹¹ is a C₁ to C₂₂ aliphatic group which can be an alkyl or alkenyl.

23. A composition according to claim 1 wherein said vinyl ether of alcohols containing 1 to 22 carbon atoms is selected from at least one compound represented by the formula: 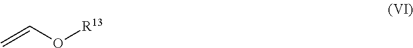
wherein R¹³ is a C₁ to C₂₂ alkyl.

24. A composition according to claim 1 wherein said associative monomer comprises (i) an ethylenically unsaturated end group portion; (ii) a polyoxyalkylene mid-section portion, and (iii) a hydrophobic end group portion containing 8 to 30 carbon atoms.

25. A composition according to claim 24 wherein said associative monomer is represented by formulas VII and/or VIIA: 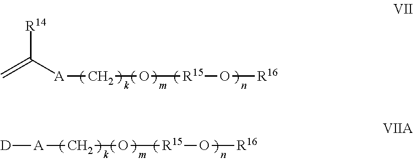
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; Y is —R¹⁵O—, —R¹⁵NH—, —C(O)—, —C(O)NH—, —R¹⁵NHC(O)NH—, or —C(O)NHC(O)—; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an araalkyl substituted phenyl, and an aryl-substituted C₂-C₃₀ alkyl; wherein the R¹⁶ alkyl group, aryl group, phenyl group optionally comprises one or more substituents selected from the group consisting of a hydroxyl group, an alkoxyl group, benzyl group styryl group, and a halogen group.

26. A composition according to claim 24 wherein said associative monomer is represented by formula VIIB: 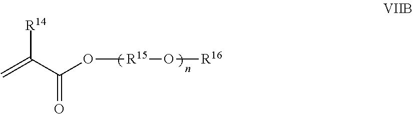
wherein R¹⁴ is hydrogen or methyl; R¹⁵ is a divalent alkylene moiety independently selected from C₂H₄, C₃H₆, and C₄H₈, and n represents an integer ranging from about 10 to about 60, (R¹⁵—O) can be arranged in a random or a block configuration; R¹⁶ is a substituted or unsubstituted alkyl selected from a C₈-C₃₀ linear alkyl, a C₈-C₃₀ branched alkyl, a C₈-C₃₀ carbocyclic alkyl, a C₂-C₃₀ alkyl-substituted phenyl, an araalkyl substituted phenyl, and an aryl-substituted C₂-C₃₀ alkyl, wherein the R¹⁶ alkyl group, aryl group, phenyl group optionally comprises one or more substituents selected from the group consisting of a hydroxyl group, an alkoxyl group, benzyl group styryl group, and a halogen group.

27. A composition according to claim 1 wherein said semi-hydrophobic monomer comprises (i) an ethylenically unsaturated end group portion; (ii) a polyoxyalkylene mid-section portion, and (iii) an end group portion selected from hydrogen or an alkyl group containing 1 to 4 carbon atoms.

28. A composition according to claim 27 wherein said semi-hydrophobic monomer is selected from at least one monomer represented by formulas VIII and IX: 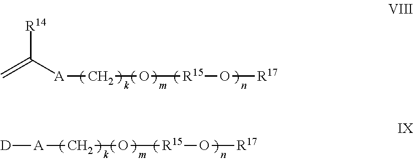
wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150; R¹⁷ is selected from hydrogen and a linear or branched C₁-C₄ alkyl group; and D represents a vinyl or an allyl moiety.

29. A composition according to claim 1 wherein said polymer is polymerized from a monomer mixture comprising at least 30 wt. % of said hydrophilic monomer(s) and at least 5 wt. % of said hydrophobic monomers.

30. A composition according to claim 1 wherein said amphiphilic polymer comprises a conventional crosslinking agent which is present in an amount sufficient to be incorporated into said polymer from about 0.01 to about 1 wt. %, based on the dry weight of the polymer.

31. A composition according to claim 30 wherein the at least one conventional crosslinking agent is selected from polyallyl ethers of trimethylolpropane, polyallyl ethers of pentaerythritol, polyallyl ethers of sucrose, or mixtures thereof.

32. A composition according to claim 30 wherein the at least one conventional crosslinking agent is selected from pentaerythritol diallyl ether, pentaerythritol triallyl ether, pentaerythritol tetraallyl ether; or mixtures thereof.

43-44. (canceled)

33. A composition according to claim 1 wherein said nonionic, amphiphilic emulsion polymer is prepared from a monomer composition comprising:
a) from about 20 to about 60 wt. % of at least one C₁-C₄ hydroxyalkyl (meth)acrylate;
b) from about 10 to about 70 wt. % of at least one C₁-C₁₂ alkyl (meth)acrylate;
c) from about 0 to about 40 wt. % of at least one vinyl ester of a C₁-C₁₀ carboxylic acid;
d) from about 0 to about 30 wt. % of a vinyl lactam;
e) from about 0 to about 15 wt. % of at least one associative and/or a semi-hydrophobic monomer (wherein all monomer weight percentages are based on the weight of the total monomers); and
f) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

34. A composition according to claim 1 wherein said nonionic, amphiphilic emulsion polymer is prepared from a monomer composition comprising:
a) from about 20 to about 60 wt. % of at least one C₁-C₄ hydroxyalkyl (meth)acrylate;
b) from about 10 to about 30 wt. % ethyl acrylate;
c) from about 10 to about 35 wt. % butyl acrylate;
d) from about 0 to about 25 wt. % of a vinyl ester of a carboxylic acid selected from vinyl formate, vinyl acetate, vinyl propionate, vinyl butyrate, vinyl isobutyrate, and vinyl valerate;
e) from about 0 to about 30 wt. % of vinyl pyrrolidone;
f) from about 0 to about 15 wt. % of at least one associative monomer and/or semi-hydrophobic monomer (wherein all monomer weight percentages are based on the weight of the total monomers); and
g) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

35. A composition according to claim 1 wherein said nonionic, amphiphilic emulsion polymer is prepared from a monomer composition comprising:
a) from about 20 to about 50 wt. % of hydroxyethyl methacrylate;
b) from about 10 to about 30 wt. % ethyl acrylate;
c) from about 10 to about 30 wt. % butyl acrylate;
d) from about 0 to about 25 wt. % of vinyl pyrrolidone;
e) from about 0 to about 25 wt. % of vinyl acetate;
f) from about 0 to about 10 wt. % of at least one associative and/or semi-hydrophobic monomer (wherein all monomer weight percentages are based on the weight of the total monomers); and
g) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

36. A composition according to claim 1 wherein said nonionic, amphiphilic emulsion polymer is prepared from a monomer composition comprising:
a) from about 20 to about 50 wt. % of hydroxyethyl methacrylate;
b) from about 10 to about 40 wt. % ethyl acrylate;
c) from about 10 to about 20 wt. % butyl acrylate;
d) from about 0.1 to about 10 wt. % of at least one associative and/or semi-hydrophobic monomer (wherein all monomer weight percentages are based on the weight of the total monomers); and
e) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

37. A composition according to claim 1 wherein said nonionic, amphiphilic emulsion polymer is prepared from a monomer composition comprising:
a) from about 20 to about 50 wt. % of hydroxyethyl methacrylate;
b) from about 10 to about 30 wt. % ethyl acrylate;
c) from about 10 to about 30 wt. % butyl acrylate;
d) from about 1 to about 10 wt. % of at least one associative and/or semi-hydrophobic monomer (wherein all monomer weight percentages are based on the weight of the total monomers); and
e) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

38. A composition according to claim 1 wherein said nonionic, amphiphilic emulsion polymer is prepared from a monomer composition comprising:
a) from about 20 to about 35 wt. % of hydroxyethyl methacrylate;
b) from about 10 to about 30 wt. % ethyl acrylate;
c) from about 10 to about 30 wt. % butyl acrylate;
d) from about 15 to about 25 wt. % of vinyl pyrrolidone,
e) from about 15 to about 25 wt. % of vinyl acetate (wherein all monomer weight percentages are based on the weight of the total monomers); and
f) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

39. A composition according to claim 1 wherein said nonionic, amphiphilic emulsion polymer is prepared from a monomer composition comprising:
a) from about 20 to about 40 wt. % of hydroxyethyl methacrylate;
b) from about 10 to about 30 wt. % ethyl acrylate;
c) from about 10 to about 30 wt. % butyl acrylate;
d) from about 15 to about 25 wt. % of vinyl pyrrolidone;
e) from about 1 to about 5 wt. % of at least one associative and/or semi-hydrophobic monomer (wherein all monomer weight percentages are based on the weight of the total monomers); and
e) from about 0.01 to about 5 wt. % of at least one crosslinker (based on the dry weight of the polymer) selected from an amphiphilic crosslinking agent or a combination of an amphiphilic crosslinking agent and said conventional crosslinking agent.

52-53. (canceled)

40. A composition according to claim 1 wherein said composition further comprises a conditioning agent selected from a cationic compound, a cationic polymer, an ampholytic polymer, a silicone, a hydrocarbon oil, a natural oil, a natural wax, a synthetic wax, and combinations thereof.

41. A composition according to claim 1 wherein said composition is selected from shampoos, baby shampoos, body washes, shower gels, liquid hand soaps, pet cleansing product, or facial cleansers.

42. A method for enhancing the phase stability of an antidandruff shampoo composition comprising adding thereto a nonionic, amphiphilic emulsion, emulsion polymer as set forth in claim 1.
